(19) United States
(12) Patent Application Publication
(10) Pub. No.: US 2016/0184391 A1
(43) Pub. Date: Jun. 30, 2016

(54) TREATMENT OF GRAFT REJECTION BY ADMINISTERING A COMPLEMENT INHIBITOR TO AN ORGAN PRIOR TO TRANSPLANT

(71) Applicant: ALEXION PHARMACEUTICALS, INC., Cheshire, CT (US)

(72) Inventors: Yi WANG, Woodbridge, CT (US); Zhao Xue YU, Cheshire, CT (US)

(21) Appl. No.: 14/910,408

(22) PCT Filed: Aug. 15, 2014
(86) PCT No.: PCT/US2014/051323

§ 371 Date: Feb. 5, 2016

Related U.S. Application Data
(60) Provisional application No. 61/867,009, filed on Aug. 16, 2013.

(51) Int. Cl. A61K 38/17; A01N 1/02; C07K 16/18

(52) U.S. Cl. CPC A61K 38/177; C07K 16/18; A01N 1/0226

(57) ABSTRACT

Methods of prolonging survival of a transplanted organ, as well as methods of preventing or attenuating rejection of a transplanted organ are provided. These methods involve contacting the organ with an inhibitor of complement activity (e.g., a complement inhibitor that has a maximum molecular weight of 70 kDa and/or a half-life shorter than 10 days, such as a CR2-FH fusion protein or a single chain anti-C5 antibody), prior to transplantation The methods also include administering to the allotransplant recipient an inhibitor of complement activity together with one or more immunosuppressants. A pretreatment with an alternative complement inhibitor was found to be effective in improving graft survival and decreasing ischemia-reperfusion injury in animal.

### BACKGROUND

Organ transplantation is the preferred treatment for most patients with chronic organ failure. Although kidney, liver, lung, and heart transplantations offer excellent opportunities for rehabilitation as recipients return to a more normal lifestyle, their application is limited by the medical/surgical suitability of potential recipients, an increasing shortage of donors, and premature failure of transplanted organ function.

Transplantation of cells, tissues and organs has become common and is often a life-saving procedure. Organ transplantation is the preferred treatment for most patients with chronic organ failure. Despite great improvement in treatments to inhibit rejection, rejection continues to be the single largest impediment to successful organ transplantation. Rejection includes not only acute rejection but also chronic rejection. One-year survival rates for transplanted kidneys average 88.3% with kidneys from deceased donors and 94.4% with kidneys received from living donors. The corresponding five-year survival rates for the transplanted kidneys are 63.3% and 76.5% (OPTN/SRTR Annual Report, 2002). The one-year survival rates are 80.2% and 76.5% for livers from deceased and living donors, respectively. The corresponding five-year liver graft survival rates are 63.5% and 73.0% (OPTN/SRTR Annual Report, 2002). The use of immunosuppressant drugs, especially cyclosporin A, and more recently tacrolimus, has dramatically improved the success rate of organ transplantation, especially by preventing acute rejection. As the numbers above show, there is still a need to improve the success rates of transplantation, both short-term and long-term. As seen from the above numbers for kidney and liver transplants, the five-year failure rates for these transplanted organs are on the order of 25-35%. In the year 2001 alone, more than 23,000 patients received an organ transplant, of which approximately 19,000 received a kidney or liver transplant (OPTN/SRTR Annual Report, 2002). Based on present techniques, it would be estimated that approximately 5,000-6,000 of these transplanted kidneys and livers will fail within 5 years. These numbers do not include other transplanted organs or transplanted tissues or cells, such as bone marrow.

There are multiple types of transplants. These are described, e.g., in Abbas et al., 2000. A graft transplanted from one individual to the same individual is called an autologous graft or autograft. A graft transplanted between two genetically identical or syngeneic individual is called a syngeneic graft. A graft transplanted between two genetically different individuals of the same species is called an allogeneic graft or allograft. A graft transplanted between individuals of different species is called a xenogeneic graft or xenograft. The molecules that are recognized as foreign on allografts are called alloantigens and those on xenografts are called xenoantigens. The lymphocytes or antibodies that react with alloantigens or xenoantigens are described as being alloreactive or xenoreactive, respectively.

Currently more than 40,000 kidney, heart, lung, liver and pancreas transplants are performed in the United States each year (Abbas et al., 2000). Other possible transplants include, but are not limited to, vascular tissue, eye, cornea, lens, skin, bone marrow, muscle, connective tissue, gastrointestinal tissue, nervous tissue, bone, stem cells, islets, cartilage, hepatocytes, and hematopoietic cells. Unfortunately, there are many more transplant candidates than there are donors. To overcome this shortage, a major effort is being made to learn how to use xenografts. While progress is being made in this field, most transplants are allografts. An allogeneic transplant, while presently being more likely to be successful than a xenogeneic transplant, must surmount numerous obstacles to be successful. There are several types of immunological attacks made by the recipient against the donor organ which can lead to rejection of the allograft. These include hyperacute rejection, acute vascular rejection (including accelerated humoral rejection and de novo acute humoral rejection), and chronic rejection. Rejection is normally a result of T-cell mediated or humoral antibody attack, but may include additional secondary factors, such as the effects of complement and cytokines.

An ever growing gap between the number of patients requiring organ transplantation and the number of donor organs available has become a major problem throughout the world (Park et al., 2003). Individuals who have developed anti-HLA antibodies are said to be immunized or sensitized (Gloor, 2005). HLA sensitization is the major barrier to optimal utilization of organs from living donors in clinical transplantation (Warren et al., 2004) due to the development of severe antibody-mediated rejection (ABMR). For example, more than 50% of all individuals awaiting kidney transplantation are presensitized patients (Glotz et al., 2002) who have elevated levels of broadly reactive alloantibodies, resulting from multiple transfusions, prior failed allografts, or pregnancy (Kupiec-Weglinski, 1996). The study of ABMR is currently one of the most dynamic areas in transplantation, due to recognition that this type of rejection can lead to either acute or chronic loss of allograft function (Mehra et al., 2003). Numerous cases of ABMR, including hyperacute rejection (HAR) or accelerated humoral rejection (ACHR), have been reported that are characterized by acute allograft injury that is resistant to potent anti-T cell therapy, the detection of circulating donor-specific antibodies, and the deposition of complement components in the graft. ABMR with elevated circulating alloantibodies and complement activation occurs in 20-30% of acute rejection cases and results in a poorer prognosis in patients relative to those with cellular rejection (Mauiyyedi et al., 2002).

Highly presensitized patients exhibiting high levels of alloantibodies usually suffer immediate and aggressive HAR. In clinical practice, owing to great efforts and significant advances in technology, HAR may be avoided by obtaining a pretransplant lymphocytotoxic cross-match to identify sensitized patients with antibodies specific for donor HLA antigens. However, circulating antibodies against donor HLA or other non-MHC endothelial antigens may also be responsible for a delayed form of acute humoral rejection, which is associated with an increased incidence of graft loss (Collins et al., 1999). Therefore, development of a novel presensitized animal model to mimic ABMR in clinical settings would be beneficial to studies on its mechanism, and to efforts toward the much-needed progress in the management of allograft rejection in presensitized hosts.

Some highly presensitized patients can benefit from intervention programs, such as those involving immunoadsorption (Palmer et al., 1989; Ross et al., 1993; Kriaa et al., 1995), plasmapheresis, or intravenous immunoglobulin (Sonnenday et al., 2002; Rocha et al., 2003) that have been designed and implemented to temporarily eliminate anti-donor antibodies. However, in addition to their benefits, the aforementioned therapies carry with them numerous drawbacks as some individuals are less susceptible to their effects (Kriaa et al., 1995; Hakim et al., 1990; Glotz et al., 1993; Tyan et al., 1994) and they are extremely expensive, time-consuming, and risky (Salama et al., 2001). Moreover, the transient and variable effect of these protocols has limited their impact (Glotz et al., 2002; Kupin et al., 1991; Schweitzer et al., 2000). Therefore, developing novel strategies to reduce the risk and cost in prevention of ABMR would be beneficial to presensitized recipients receiving a graft (e.g., an allograft).

Complement pathways have been known to play an important role in ischemia-reperfusion injury in organ transplantations. For a review on complement in transplantation, see, e.g., Baldwin et al., 2003, and Chowdbury et al., 2003. Inhibiting complement activation has been proposed to improve graft survival but most believe that it is necessary to treat the recipient with a complement inhibitor prior to transplantation and/or that an inhibition to both classical and alternative complement pathways, or to terminal complement components (e.g., the MAC complex), is needed. For an example on treating ischemia-reperfusion injury with a complement inhibitor antagonizing both classical and alternative complement pathways, see, e.g., Wada et al., 2001 and de Vries et al., 2003. Due to multiple endogenous rejection mechanisms towards the transplanted organ, more studies on complement inhibition treatment are needed to confirm its overall therapeutic effect in transplantation.

### SUMMARY OF THE INVENTION

Provided are methods and compositions for prolonging the survival of a graft (e.g., an allograft) in a mammal.

Accordingly, in one aspect, the invention provides methods to prolong survival of an organ that is transplanted from a donor mammal to a recipient mammal, as well as methods to prevent or attenuate rejection (e.g., hyperacute rejection, antibody-mediated rejection, or chronic rejection) of a transplanted organ in a recipient mammal, which involve administering a complement inhibitor to the organ prior to transplantation, wherein the complement inhibitor has a maximum molecular weight of 70 kDa and/or a half-life of less than 10 days. Such inhibitors can act via either the classical or alternative complement pathway, or both pathways. Particular complement inhibitors for use in the invention include, for example, TT30, TT32 or a single chain anti-C5 antibody, such as pexelizumab or a single chain version of eculizumab or an Fab of eculizumab.

In another aspect, the invention provides methods to prolong survival of an organ that may be transplanted from a donor mammal to a recipient mammal, which include administering an alternative complement pathway inhibitor to the organ prior to transplantation. The organ may be contacted with a solution that includes an inhibitor of complement or terminal complement, following removal of the organ from the donor mammal, but prior to the transplant. In one embodiment, the organ is perfused with or soaked in the solution for 0.5 to 60 hours, such as 1-30 hours or 28 hours. In one embodiment, another embodiment, the solution may be removed and, subsequently, the organ may be reperfused with or soaked in a second solution that does not include an inhibitor of complement or terminal complement. In particular embodiments, the period of reperfusion with the second liquid may be 0.25 to 3 hours, such as 2 hours or 0.5 hours. In any of the above embodiments involving perfusion or reperfusion, the perfusion or reperfusion may be a period of cold ischemia.

In another aspect, the invention provides a method to prolong survival of a recipient mammal after receiving an organ transplant from a donor mammal in which the method includes administering an alternative complement pathway inhibitor to the organ prior to transplantation.

In another aspect the invention provides a method to improve organ function in a recipient mammal after receiving the organ transplant from a donor mammal in which the method includes administering an alternative complement pathway inhibitor to the organ prior to transplantation.

In another aspect the invention provides a method to prevent or attenuate ischemia-reperfusion injury in a recipient mammal after receiving an organ transplant from a donor mammal in which the method includes administering an alternative complement pathway inhibitor to the organ prior to transplantation.

In another aspect the invention provides a method to prevent or attenuate hyperacute rejection in a recipient mammal after receiving an organ transplant from a donor mammal in which the method includes administering an alternative complement pathway inhibitor to the organ prior to transplantation.

In another aspect the invention provides a method to prevent or attenuate acute graft injury in a recipient mammal after receiving an organ transplant from a donor mammal in which the method includes administering an alternative complement pathway inhibitor to the organ prior to transplantation.

In another aspect the invention provides a method to prevent or attenuate delayed graft function (DGF) in a recipient mammal after receiving an organ transplant from a donor mammal in which the method includes administering an alternative complement pathway inhibitor to the organ prior to transplantation.

In another aspect the invention provides a method to prevent or attenuate antibody-mediated rejection (AMR) in a recipient mammal after receiving an organ transplant from a donor mammal in which the method includes administering an alternative complement pathway inhibitor to the organ prior to transplantation.

In another aspect the invention provides a method to prevent or attenuate chronic rejection in a recipient mammal after receiving an organ transplant from a donor mammal in which the method includes administering an alternative complement pathway inhibitor to the organ prior to transplantation.

Exemplary organs that can be used in the methods of the present invention include, but are not limited to kidney, heart, lung, pancreas, liver, vascular tissue, eye, cornea, lens, skin, bone marrow, muscle, connective tissue, gastrointestinal tissue, nervous tissue, bone, stem cells, islets, cartilage, hepatocytes, and hematopoietic cells. In one embodiment, the organ is a kidney.

In any of the above embodiments, the alternative complement pathway inhibitor can be administered to the organ after removal of the organ from the donor mammal and prior to preservation of the organ. In another embodiment, the alternative complement pathway inhibitor is administered to the organ during preservation of the organ. In these embodiments, the preservation of the organ results in cold ischemia in the organ. In certain embodiments, the alternative complement pathway inhibitor may be administered to the organ after preservation of the organ and prior to transplantation. In any of the above embodiments, the alternative complement pathway inhibitor can be administrated in conjunction with at least one immunosuppressive drug (e.g., one or more immunosuppressive drugs). In one embodiment, the immunosuppressive drug is selected from the group consisting of cyclosporin A, tacrolimus, sirolimus, OKT3, a corticosteroid, daclizumab, basiliximab, azathioprene, mycophenolate mofetil, methotrexate, 6-mercaptopurine, anti-T cell antibodies, cyclophosphamide, leflunamide, brequinar, ATG, ALG, 15-deoxyspergualin, LF15-0195, and bredinin and combinations thereof. In other embodiments, the alternative complement pathway inhibitor is administrated in conjunction with at least one additional inhibitor of the classical, alternative, or lectin complement pathway.

In any of the above embodiments, the donor mammal or recipient mammal is a human.

In any of the above embodiments, the alternative complement pathway inhibitor specifically increases the stability or function of factor H, Complement Factor H-Related proteins (CFHRs), factor I, complement receptor 1 (CR1), complement receptor 2 (CR2), MCP, DAF, CD59, CD55, CD46, Crry, and C4 binding protein. In particular embodiments, the complement inhibitor may be a factor H fusion protein. In still more particular embodiments, the factor H fusion protein may be a CR2-FH molecule. In certain embodiments, the CR2-FH molecule includes a CR2 portion including a CR2 or a fragment thereof and an FH portion including a FH or a fragment thereof, such that the CR2-FH molecule may be capable of binding to a CR2 ligand. The CR2 portion may include at least the first two N-terminal SCR domains of CR2. In some embodiments, the CR2 portion includes at least the first four N-terminal SCR domains of CR2. In certain embodiments, the FH portion includes at least the first four SCR domains of FH or at least the first five SCR domains of FH. In particular embodiments, the CR2-FH molecule may include two or more FH portions. In some embodiments, the CR2 portion includes the first two N-terminal SCR domains of CR2 and the FH portion includes the first four SCR domains of FH, while in others the CR2 portion includes the first four N-terminal SCR domains of CR2 and the FH portion includes the first five SCR domains of FH. In other embodiments, the CR2 portion includes amino acids 23 to 271 of SEQ ID NO:1 and the FH portion includes amino acids 21 to 320 of SEQ ID NO:2.

In yet a further aspect, the invention includes methods to prolong survival of an organ that is transplanted from a donor mammal to a recipient mammal, as well as methods to prevent or attenuate rejection (e.g., hyperacute rejection, antibody-mediated rejection, or chronic rejection) of a transplanted organ in a recipient mammal, which involve administering a complement inhibitor to the organ prior to transplantation, wherein the complement inhibitor has a maximum molecular weight of 70 kDa and/or a half-life of less than 10 days. Such inhibitors can act via either the classical or alternative complement pathway, or both pathways. Particular complement inhibitors for use in the invention include, for example, TT30, TT32 or a single chain anti-C5 antibody, such as pexelizumab or a single chain version of eculizumab or an Fab of eculizumab.

Suitable complement inhibitors typically have a molecular weight of less than 70 kDa, less than 69 kDa, less than 68 kDa, less than 67 kDa, less than 66 kDa, less than 65 kDa, less than 64 kDa, less than 63 kDa, less than 62 kDa, less than 61 kDa, less than 60 kDa, less than 59 kDa, less than 58 kDa, less than 57 kDa, less than 56 kDa, less than 55 kDa, less than 54 kDa, less than 53 kDa, less than 52 kDa, less than 51 kDa, less than 50 kDa, less than 49 kDa, less than 48 kDa, less than 47 kDa, less than 46 kDa, less than 45 kDa, less than 43 kDa, less than 42 kDa, less than 41 kDa, less than 40 kDa, less than 39 kDa, less than 38 kDa, less than 37 kDa, less than 36 kDa, less than 35 kDa, less than 34 kDa, less than 33 kDa, less than 32 kDa, less than 31 kDa, less than 30 kDa, less than 29 kDa, less than 28 kDa, less than 27 kDa, less than 26 kDa, less than 25 kDa, less than 24 kDa, less than 23 kDa, less than 22 kDa, less than 21 kDa, less than 20 kDa, or less than 19 kDa). In one embodiment, the complement inhibitor has a molecular weight of about 64-66 kDa. In another embodiment, the complement inhibitor has a molecular weight of or about 65 kDa. In another embodiment, the complement inhibitor has a molecular weight of about 26-27 kDa. In another embodiment, the complement inhibitor has a molecular weight of or about 26 kDa. In a particular embodiment, the complement inhibitor has a molecular weight of or about 26.28 kDa or 26.25 kDa.

Additionally, suitable complement inhibitors can have a half-life less than 10 days, 9.5 days, 9 days, 8.5 days, 8 days, 7.5 days, 7 days, 6.5 days, 6 days, 5.5 days, 5 days, 4.5 days, 4 days, 3.5 days, or 3 days. In one embodiment, the complement inhibitor has a short half-life (e.g., less than 10 days) and has substantially cleared from the organ prior to transplantation into the recipient mammal.

In a particular embodiment, the complement inhibitor has both a maximum molecular weight of 70 kDa and a half-life shorter than 10 days.

Complement inhibitors having a maximum molecular weight of 70 kDa and/or a half-life of less than 10 days are advantageous because they can more easily penetrate the organ and block complement activation in the donor organ. However, due to their low molecular weights and/or short half live, they are substantially cleared from the organ prior to transplantation, thereby minimizing the impact on the recipient's innate immune responses again infection. This is particularly important since transplant recipients are typically given immunosuppressive treatment after transplantation and are, therefore, at risk for infection. Clearance of the complement inhibitor from the donor organ is further advantageous because the recipient will not require prior vaccination for Neisseria meningitidi before receiving the donor organ.

In one embodiment, the complement inhibitor is a fusion protein comprising a complement receptor 2 (CR2) fragment linked to a complement inhibitory domain of complement factor H (CFH). In another embodiment, the complement inhibitor is a human CR2-FH fusion protein comprising SEQ ID NO:3. In a particular embodiment the complement inhibitor is TT30 (also known as ALXN1102).

In another embodiment, the complement inhibitor is a single chain antibody, e.g., single chain an anti-C5 antibody. In one embodiment, the single chain anti-C5 comprises SEQ ID NO:27. In another embodiment, the single chain anti-C5 comprises SEQ ID NO:29. In a particular embodiment, the single chain anti-C5 antibody is a single chain version of eculizumab. In another particular embodiment, the single chain anti-C5 antibody is pexelizumab.

In another embodiment, the complement inhibitor is a Fab comprising the VH-CH1 of the heavy chain (SEQ ID NO:30) VL-CL of the light chain (SEQ ID NO: 31) of anti-C5 antibody eculizumab.

In one embodiment, the anti-C5 antibody comprises the heavy and light chain complementarity determining regions (CDRs) or variable regions (VRs) of eculizumab. In another embodiment, the anti-C5 antibody comprises a heavy chain comprising the amino acid sequence set forth in SEQ ID NO:1. In another embodiment, the anti-C5 antibody comprises a light chain comprising the amino acid sequence set forth in SEQ ID NO:2. In another embodiment, the anti-C5 antibody comprises heavy and light chains comprising the amino acid sequences set forth in SEQ ID NOs: 1 and 2, respectively.

The complement inhibitor is administered to the organ prior to transplantation (e.g., after removal of the organ from a donor mammal and before transplant of the organ into a recipient mammal). In one embodiment, the complement inhibitor is administered at an organ procurement center. In another embodiment, the complement inhibitor is administered immediately prior to transplantation, e.g., in a “back table” procedure within hours or minutes prior to translation.

The complement inhibitor can be administered to the organ by any suitable technique. In one embodiment, the complement inhibitor is administered to the organ by perfusing the organ with a solution containing the complement inhibitor. In another embodiment, the organ is bathed in a solution containing the complement inhibitor. In one embodiment, the organ is perfused with or soaked in a solution containing the complement inhibitor for 0.5 hours to 60 hours or for 1 hour to 30 hours (e.g., for 30 minutes, 35 minutes, 40 minutes, 45 minutes, 50 minutes, 55 minutes, 1 hour, 1.5 hours, 2 hours, 2.5 hours, 3 hours, 3.5 hours, 4 hours, 4.5 hours, 5 hours, 5.5 hours, 6 hours, 6.5 hours, 7 hours, 7.5 hours, 8 hours, 8.5 hours, 9 hours, 9.5 hours, 10 hours, 10.5 hours, 11 hours, 11.5 hours, 12 hours, 12.5 hours, 13 hours, 13.5 hours, 14 hours, 14.5 hours, 15 hours, 15.5 hours, 16 hours, 16.5 hours, 17 hours, 17.5 hours, 18 hours, 18.5 hours, 19 hours, 19.5 hours, 20 hours, 21 hours, 22 hours, 23 hours, 24 hours, 25 hours, 26 hours, 27 hours, 28 hours, 29 hours, or 30 hours).

In one embodiment, the recipient mammal is not vaccinated (e.g., against Neisseria meningitides) prior to transplantation. In another embodiment, the recipient is not treated with a complement inhibitor after transplantation.

Exemplary organs that can be used in the methods of the present invention include, but are not limited to kidney, heart, lung, pancreas, liver, vascular tissue, eye, cornea, lens, skin, bone marrow, muscle, connective tissue, gastrointestinal tissue, nervous tissue, bone, stem cells, islets, cartilage, hepatocytes, and hematopoietic cells.

### BRIEF DESCRIPTION OF THE DRAWINGS

FIG. 1 provides schematic diagrams of an exemplary CR2-FH expression plasmid and CR2-FH proteins. For the CR2-FH expression plasmid, k refers to Kozak sequence, 5 refers to CD5 signal peptide, 1 refers to an optional linker, s refers to stop codon and polyA signal. For the CR2-FH proteins (with or without signal peptide), 5 refers to the CD5 signal peptide, 1 refers to an optional linker.

FIG. 2 provides the amino acid sequence of human CR2 (SEQ ID NO:1) and the amino acid sequence of human factor H (SEQ ID NO:2).

FIG. 3 provides the amino acid sequence of an exemplary human CR2-FH fusion protein (SEQ ID NO: 3) and an exemplary polynucleotide sequence encoding a human CR2-FH fusion protein (SEQ ID NO:4).

FIGS. 4-6 provide exemplary amino acid sequences of CR2-FH molecules described herein (SEQ ID NOs: 5-10). “nnn” represents an optional linker.

FIG. 7 provides exemplary amino acid sequences of signaling peptides described herein (SEQ ID NOs:11, 13, and 25) and exemplary polynucleotide sequences encoding the signaling peptides (SEQ ID NOs:12, 14, and 26).

FIG. 8 provides the amino acid sequence of mouse CR2 (SEQ ID NO:15) and amino acid sequence of mouse factor H (SEQ ID NO:16).

FIG. 9 provides the amino acid sequence of an exemplary mouse CR2-FH fusion protein (SEQ ID NO:17) and an exemplary polynucleotide sequence that encodes a mouse CR2-FH plus the signal peptide (SEQ ID NO:18).

FIG. 10 provides an exemplary DNA sequence of CR2NLFHFH, a mouse CR2-FH fusion protein containing a CR2 portion and two FH portions without a linker sequence (SEQ ID NO:19).

FIG. 11 provides an exemplary DNA sequence of CR2LFHFH, a mouse CR2-FH fusion protein containing a CR2 portion linked to two FH portions via a linker sequence (SEQ ID NO:20).

FIG. 12 provides an amino acid sequence of an exemplary human CR2-FH fusion protein (designated as human CR2-fH or CR2fH) (SEQ ID NO:21) and an exemplary polynucleotide sequence that encodes a human CR2-fH plus the signal peptide (SEQ ID NO:22). The sequence encoding the signal peptide is underlined.

FIG. 13 provides an exemplary amino acid sequence of a human CR2-FH fusion protein containing two FH portions (designated as human CR2-FH2 or human CR2fH2) (SEQ ID NO:23) and an exemplary polynucleotide sequence that encodes a human CR2-FH2 plus the signal peptide (SEQ ID NO:24). The sequence encoding the signal peptide is underlined.

FIG. 14 shows the inhibition of the classical complement pathway by an anti-rat C5 monoclonal antibody (18A10) and the inhibition of the alternative complement pathway by hTT30 (human CR2-FH) in an in vitro red blood cell lysis assay.

FIG. 15 provides an exemplary method for rat kidney transplant. Complement inhibitors (e.g., anti-C5 mAb or hTT30) or control were used to treat the kidney prior to transplantation.

FIG. 16 shows the percentage of animal survival after renal transplantation with or without complement inhibitor pretreatment (either anti-C5 mAb or hTT30).

FIG. 17 shows the blood creatinine (17B) and BUN (17A) levels in the recipient animal, with or without complement inhibitor pretreatment (either anti-C5 mAb or hTT30), at Day 3 post-transplantation.

FIG. 18 shows the histological image of the transplanted kidney at Day 3 or 21 post-transplantation for normal and complement inhibitor pretreated (either anti-C5 mAb or hTT30) animals.

FIG. 19A is a schematic depicting the experimental procedure, i.e., organ perfusion with TT30 immediately prior to transplantation. FIG. 19B is graph showing the percent survival of recipient mice wherein TT30 or 18A10 was administered to the organ prior to transplant.

FIG. 20 is a graph showing C3 concentrations in rat kidney lysates, wherein the donor organ was perfused twice with TT30.

FIG. 21 is a schematic depicting the sequence of single chain pexelizumab. As shown in FIG. 21, single chain eculizumab and single pexelizumab differ at position 38 (i.e., single chain eculizumab has a glutamine residue at position 38, whereas pexelizumab has an arginine residue at position 38).

FIG. 22 is a schematic depicting the sequence of single chain eculizumab. As shown in FIG. 21, single chain eculizumab and single pexelizumab differ at position 38 (i.e., single chain eculizumab has a glutamine residue at position 38, whereas pexelizumab has an arginine residue at position 38).

FIG. 23 is a schematic depicting the sequence of TT30, which distinguishes the CR2 and Factor H portions.

FIG. 24 is a schematic representation of the SCR Domains of TT30 as related to Factor H (white) and CR2 (black).

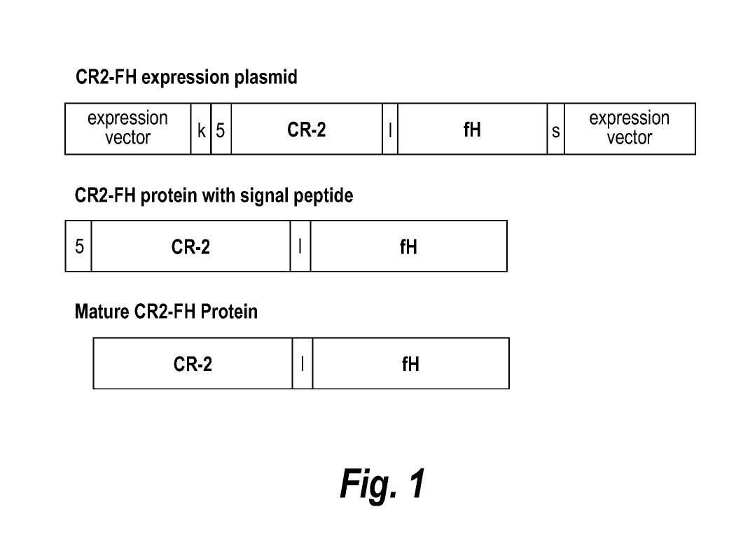

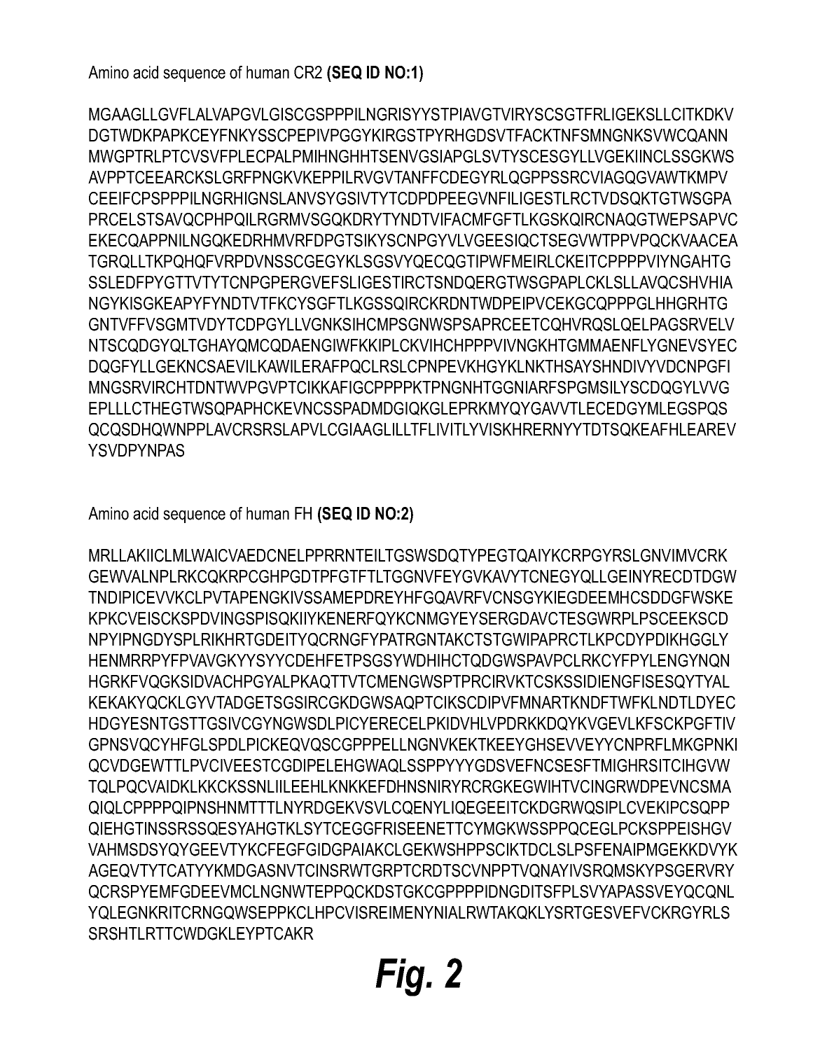

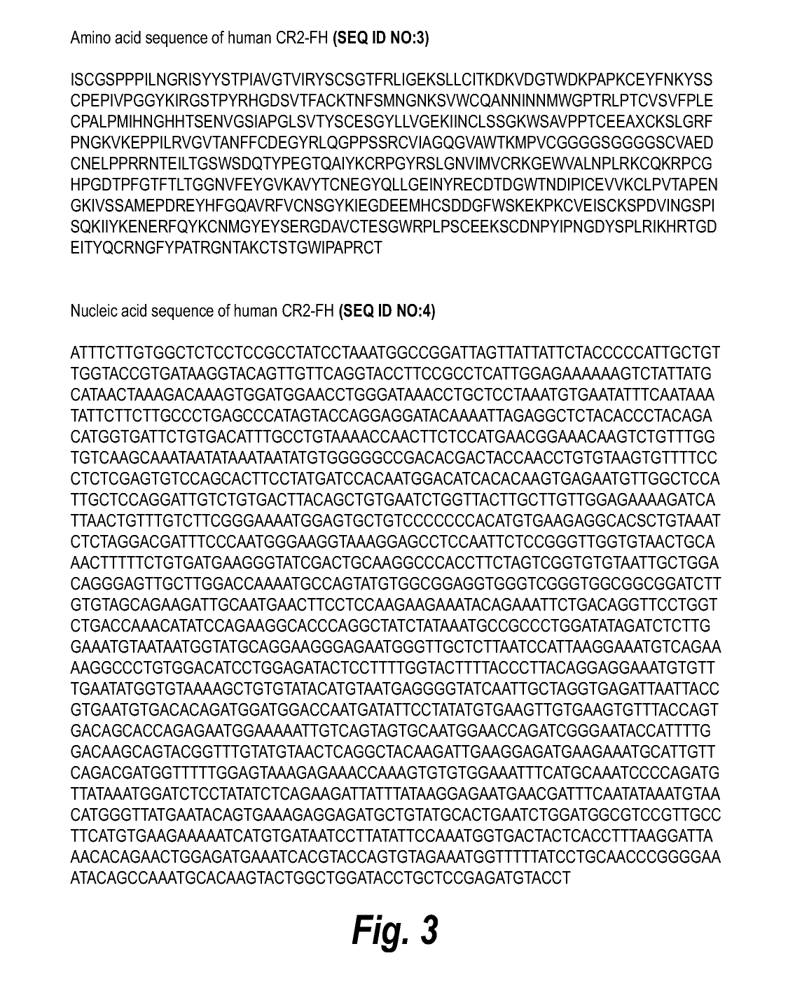

### DETAILED DESCRIPTION

As used herein, the term “organ” refers to any cell, tissue, or organ for transplantation. Exemplary organs include, but are not limited to kidney, heart, lung, pancreas, liver, vascular tissue, eye, cornea, lens, skin, bone marrow, muscle, connective tissue, gastrointestinal tissue, nervous tissue, bone, stem cells, islets, cartilage, hepatocytes, and hematopoietic cells. In a particular embodiment, the organ is a kidney.

As used herein, the term “transplant” refers to the replacement of an organ in a human or non-human animal recipient. The purpose of replacement is to remove a diseased organ or tissue in the host and replace it with a healthy organ or tissue from the donor. Where the donor and the recipient are the same species the transplant is known as an allograft. Where the donor and the recipient are dissimilar species the transplant is known as a xenograft. The techniques necessary for transplantation are varied and depend to a large extent on the nature of the organ being transplanted. The success of the transplant as a therapeutic modality depends on a number of possible physiological outcomes.

As used herein, the term “perfusion” refers to the passage of a fluid through a specific organ or an area of the body. Stated another way, perfusion or to “perfuse” refers to supplying an organ, tissue with a fluid by circulating it through blood vessels or other natural channels. Techniques for perfusing organs and tissue are well known in the art, and are disclosed in International Patent Application WO2011/002926, and U.S. Pat. Nos. 5,723,282 and 5,699,793 which are both incorporated herein in their entirety by reference.

As used herein, the term “solution” refers to any fluid capable of comprising a complement inhibitor.

As used herein the terms “attenuate” and “prevent” refer to a decrease by a statistically significant amount. For example, in one embodiment, attenuating or preventing refers to either partially or completely inhibiting rejection In one embodiment, “attenuating” means a decrease by at least 10% compared to a reference level, for example a decrease by at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or up to and including a 100% decrease compared to a reference sample, or any decrease between 10-100% compared to a reference level.

As used herein the term “prolong” refer to an increase by a statistically significant amount. For example, in one embodiment, prolonging survival of a graft refers to increasing the survival of a graft, e.g., by at least 10% compared to a reference level, for example a decrease by at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or up to and including a 100% increase compared to a reference sample, or any increase between 10-100% compared to a reference level.

As used herein, the terms “treating” or “to treat” a disease or disorder is defined as administering one or more complement inhibitors, with or without other therapeutic agents, in order to palliate, ameliorate, stabilize, reverse, slow, delay, prevent, reduce, or eliminate the disease or disorder or a symptom of the disease or disorder, or to retard or stop the progression of the disease or disorder or a symptom of the disease or disorder. An “effective amount” is an amount sufficient to treat a disease or disorder, as defined above.

An “individual” is a vertebrate, preferably a mammal, more preferably a human. Mammals include, but are not limited to, farm animals, sport animals, pets, primates, mice and rats. In some embodiments, the individual is human. In some embodiments, the individual is an individual other than human. In some embodiments, the individual is an animal model for the study of a disease in which the alternative complement pathway is implicated. Individuals amenable to treatment include those who are presently asymptomatic but who are at risk of developing a symptomatic macular degeneration-related disorder at a later time. For example, human individuals include those having relatives who have experienced such a disease, and those whose risk is determined by analysis of genetic or biochemical markers, by biochemical methods, or by other assays such as T cell proliferation assay. In some embodiments, the individual is a human having a mutation or polymorph in its FH gene that indicates an increased susceptibility to develop a disease in which alternative complement pathway is implicated (such as age-related macular degeneration). In some embodiments, the individual has a wildtype or protective haplotype of FH. Different polymorphs of FH have been disclosed in US Pat. Pub. No. 20070020647, which is incorporated herein in its entirety.

### Rejection

As used here, the term “rejection” refers to the process or processes by which the immune response of an organ transplant recipient mounts a reaction against the transplanted organ, cell or tissue, sufficient to impair or destroy normal function of the organ. The immune system response can involve specific (antibody and T cell-dependent) or non-specific (phagocytic, complement-dependent, etc.) mechanisms, or both.

“Hyperacute rejection” occurs within minutes to hours after transplant and is due to preformed antibodies to the transplanted tissue antigens. It is characterized by hemorrhage and thrombotic occlusion of the graft vasculature. The binding of antibody to endothelium activates complement, and antibody and complement induce a number of changes in the graft endothelium that promote intravascular thrombosis and lead to vascular occlusion, the result being that the grafted organ suffers irreversible ischemic damage (Abbas et al., 2000). Hyperacute rejection is often mediated by preexisting IgM alloantibodies, e.g., those directed against the ABO blood group antigens expressed on red blood cells. This type of rejection, mediated by natural antibodies, is the main reason for rejection of xenotransplants. Hyperacute rejection due to natural IgM antibodies is no longer a major problem with allografts because allografts are usually selected to match the donor and recipient ABO type. Hyperacute rejection of an ABO-matched allograft may still occur, usually mediated by IgG antibodies directed against protein alloantigens, such as foreign MHC molecules, or against alloantigens expressed on vascular endothelial cells. Such antibodies may arise as a result of prior exposure to alloantigens through blood transfusion, prior transplantation, or multiple pregnancies (this prior exposure being referred to as “presensitization”; Abbas et al., 2000).

“Acute rejection” is a process of vascular and parenchymal injury mediated by T cells, macrophages, and antibodies that usually begins after the first week of transplantation (Abbas et al., 2001). T lymphocytes play a central role in acute rejection by responding to alloantigens, including MHC molecules, present on vascular endothelial and parenchymal cells. The activated T cells cause direct lysis of graft cells or produce cytokines that recruit and activate inflammatory cells, which cause necrosis. Both CD4⁺ and CD8⁺ cells may contribute to acute rejection. The destruction of allogeneic cells in a graft is highly specific and a hallmark of CD8⁺ cytotoxic T lymphocyte killing (Abbas et al., 2000). CD4⁺ T cells may be important in mediating acute graft rejection by secreting cytokines and inducing delayed-type hypersensitivity-like reactions in grafts, with some evidence available that indicates that CD4⁺ T cells are sufficient to mediate acute rejection (Abbas et al., 2000). Antibodies can also mediate acute rejection after a graft recipient mounts a humoral immune response to vessel wall antigens and the antibodies that are produced bind to the vessel wall and activate complement (Abbas et al., 2000).

“Delayed graft function” is a form of acute transplant failure resulting in post-transplantation oliguria, increased allograft immunogenicity and risk of acute rejection episodes, and decreased long-term survival. Factors related to the donor, the transplant, and the recipient can contribute to this condition. For a review of delayed graft function, see, e.g., Perico et al., 2004. Lancet, 364:1814-27.

“Chronic rejection” is characterized by fibrosis with loss of normal organ structures occurring over a prolonged period. The pathogenesis of chronic rejection is less well understood than that of acute rejection. Graft arterial occlusion may occur as a result of the proliferation of intimal smooth muscle cells (Abbas et al., 2000). This process is called accelerated or graft arteriosclerosis and can develop in any vascularized organ transplant within 6 months to a year after transplantation.

“Antibody-mediated rejection (ABMR)” is another type of rejection and remains the primary obstacle in kidney transplantation for highly sensitized patients.

For a transplant to be successful, the several modes of rejection must be overcome. Multiple approaches are utilized in preventing rejection. This may require administration of immunosuppressants (discussed in further detail below), often several types to prevent the various modes of attack (e.g., inhibition of T-cell attack, antibodies, and cytokine and complement effects). Prescreening of donors to match them with recipients is also a major factor in preventing rejection, especially in preventing hyperacute rejection. Immunoadsorption of anti-HLA antibodies prior to grafting may reduce hyperacute rejection. Prior to transplantation, the recipient or host may be administered anti-T cell reagents, e.g., the monoclonal antibody OKT3, Anti-Thymocyte Globulin (ATG), cyclosporin A, or tacrolimus (FK 506). Additionally, glucocorticoids and/or azathioprine may be administered to the host prior to transplantationDrugs used to aid in preventing transplant rejection include, but are not limited to, ATG or ALG, OKT3, daclizumab, basiliximab, corticosteroids, 15-deoxyspergualin, LF15-0195, cyclosporins, tacrolimus, azathioprine, methotrexate, mycophenolate mofetil, 6-mercaptopurine, bredinin, brequinar, leflunamide, cyclophosphamide, sirolimus, anti-CD4 monoclonal antibodies, CTLA4-Ig, anti-CD154 monoclonal antibodies, anti-LFA1 monoclonal antibodies, anti-LFA-3 monoclonal antibodies, anti-CD2 monoclonal antibodies, and anti-CD45. For a further discussion of rejections or injuries in organ transplant, see WO2005110481, which is incorporated herein by reference to its entirety.

### Complement and Transplant/Graft Rejection

The complement system is described in detail in U.S. Pat. No. 6,355,245. The complement system acts in conjunction with other immunological systems of the body to defend against intrusion of cellular and viral pathogens. There are at least 25 complement proteins, which are found as a complex collection of plasma proteins and membrane cofactors. The plasma proteins make up about 10% of the globulins in vertebrate serum. Complement components achieve their immune defensive functions by interacting in a series of intricate but precise enzymatic cleavage and membrane-binding events. The resulting complement cascade leads to the production of products with opsonic, immunoregulatory, and lytic functions.

The complement cascade progresses via the classical pathway or the alternative pathway. These pathways share many components and, while they differ in their initial steps, they converge and share the same “terminal complement” components (C5 through C9) responsible for the activation and destruction of target cells.

The classical complement pathway is typically initiated by antibody recognition of and binding to an antigenic site on a target cell. The alternative pathway is usually antibody independent and can be initiated by certain molecules on pathogen surfaces. Both pathways converge at the point where complement component C3 is cleaved by an active protease (which is different in each pathway) to yield C3a and C3b. Other pathways activating complement attack can act later in the sequence of events leading to various aspects of complement function.

### Complement Inhibitors

Any suitable complement inhibitor having a low molecular weight and/or a half-life of less than 10 days can be used in the methods of the present invention.

As used herein, the phrase “molecular weight” refers to the sum of the atomic weights of the atoms contained in a molecule. For example, the complement inhibitor can have a molecular weight less than 70 kDa, less than 69 kDa, less than 68 kDa, less than 67 kDa, less than 66 kDa, less than 65 kDa, less than 64 kDa, less than 63 kDa, less than 62 kDa, less than 61 kDa, less than 60 kDa, less than 59 kDa, less than 58 kDa, less than 57 kDa, less than 56 kDa, less than 55 kDa, less than 54 kDa, less than 53 kDa, less than 52 kDa, less than 51 kDa, less than 50 kDa, less than 49 kDa, less than 48 kDa, less than 47 kDa, less than 46 kDa, less than 45 kDa, less than 43 kDa, less than 42 kDa, less than 41 kDa, less than 40 kDa, less than 39 kDa, less than 38 kDa, less than 37 kDa, less than 36 kDa, less than 35 kDa, less than 34 kDa, less than 33 kDa, less than 32 kDa, less than 31 kDa, less than 30 kDa, less than 29 kDa, less than 28 kDa, less than 27 kDa, less than 26 kDa, less than 25 kDa, less than 24 kDa, less than 23 kDa, less than 22 kDa, less than 21 kDa, less than 20 kDa, or less than 19 kDa). In one embodiment, the complement inhibitor has a molecular weight of about 64-66 kDa. In another embodiment, the complement inhibitor has a molecular weight of or about 65 kDa. In another embodiment, the complement inhibitor has a molecular weight of about 26-27 kDa. In another embodiment, the complement inhibitor has a molecular weight of or about 26 kDa. In another embodiment, the complement inhibitor has a molecular weight of or about 26.28 kDa or 26.25 kDa. In yet a further embodiment, the complement inhibitor has a molecular weight less than the molecular weight of eculizumab (i.e., less than about 148 kDa).

As used herein, the phrase “half-life” refers to the time it takes for the plasma concentration of a complement inhibitor to reach half of its original concentration. In one embodiment, the complement inhibitor has a half-life of less than 10 days. For example, the complement inhibitor can have a half-life less than 10 days, 9.5 days, 9 days, 8.5 days, 8 days, 7.5 days, 7 days, 6.5 days, 6 days, 5.5 days, 5 days, 4.5 days, 4 days, 3.5 days, or 3 days. In one embodiment, the complement inhibitor has a short half-life (e.g., less than 10 days) and has substantially cleared from the organ prior to transplantation into the recipient mammal. In another embodiment, the complement inhibitor has a shorter half-life than eculizumab (i.e., less than about 291 hours or approximately 12.1 days).

In one embodiment the complement inhibitor is used as a component of a solution to preserve an organ as it is transferred to a new location for use in a transplant recipient. In this context “half-life” refers to the time it takes for the solution concentration of a complement inhibitor to reach half of its original concentration.

The complement inhibitor can have both a maximum molecular weight of 70 kDa and/or a half-life shorter than 10 days.

The above described inhibitors are advantageous because they can easily penetrate the organ and block complement activation in the donor organ. However, due to their low molecular weights and/or short half live, they are substantially cleared from the organ prior to transplantation, thereby minimizing the impact on the recipient's innate immune responses again infection. This is particularly important since transplant recipients are typically given immunosuppressive treatment after transplantation and are, therefore, at risk for infection.

### Single Chain Antibodies

As used herein the phrase “single chain antibody” (also known as a single-chain variable fragment (scFv)) refers to a fusion of a heavy chain variable region and a light chain variable region of an immunoglobulin, connected with a short linker peptide.

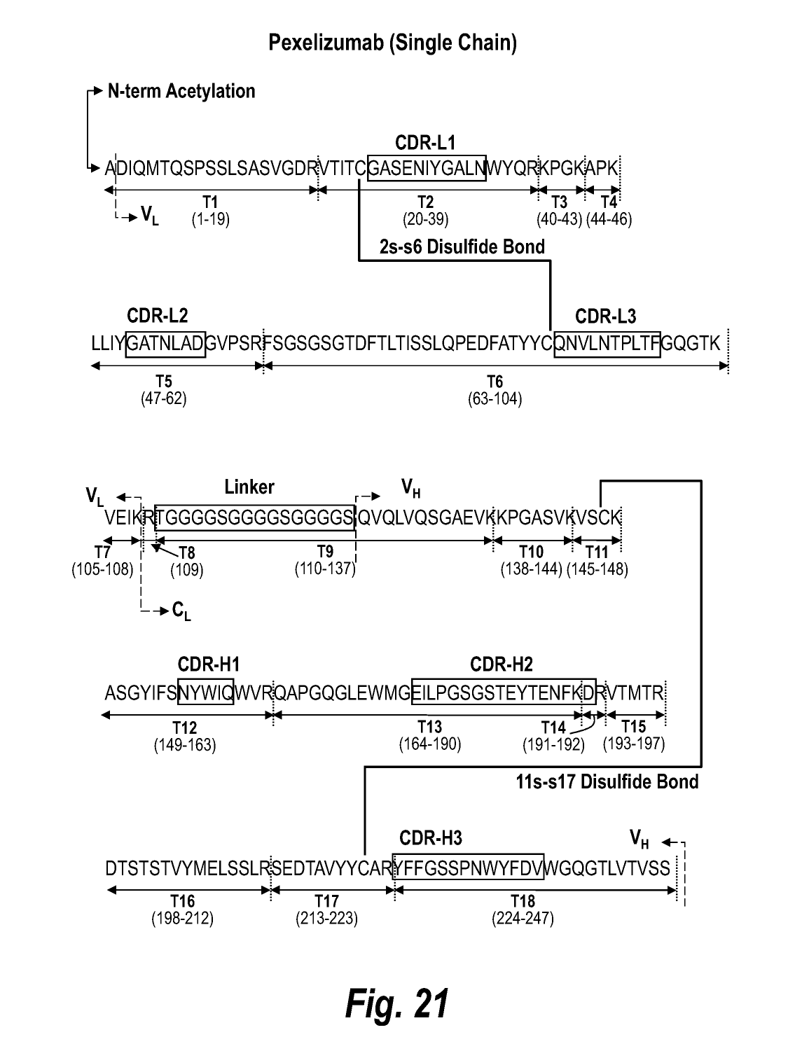

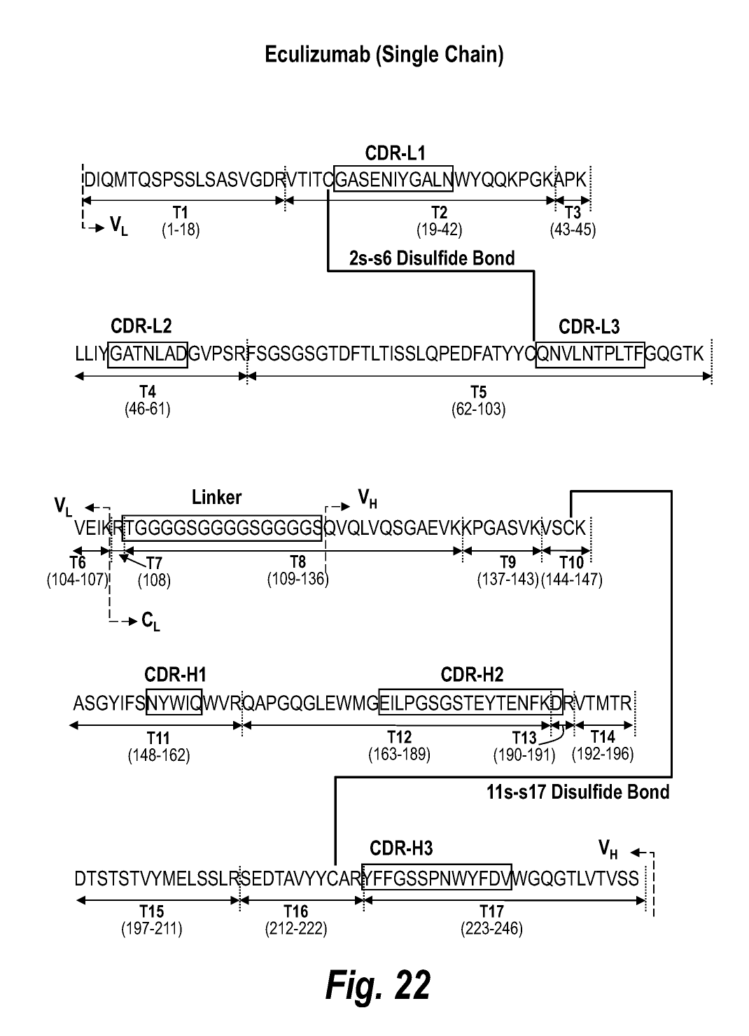

In one embodiment, the complement inhibitor is a single chain antibody, e.g., a single chain anti-C5 antibody. In one embodiment, the single chain anti-C5 comprises SEQ ID NO:27. In another embodiment, the single chain anti-C5 comprises SEQ ID NO:29. In a particular embodiment, the single chain anti-C5 antibody is a single chain version of eculizumab. The sequence of single chain eculizumab is depicted in FIG. 22. In another particular embodiment, the single chain anti-C5 antibody is pexelizumab. The sequence of single chain pexelizumab is depicted in FIG. 21.

### Fab Fragments

In another embodiment, the complement inhibitor is a Fab comprising the VH-CH1 of the heavy chain (SEQ ID NO:30) VL-CL of the light chain (SEQ ID NO: 31) of anti-C5 antibody eculizumab.

### CR2-FH Fusion Proteins

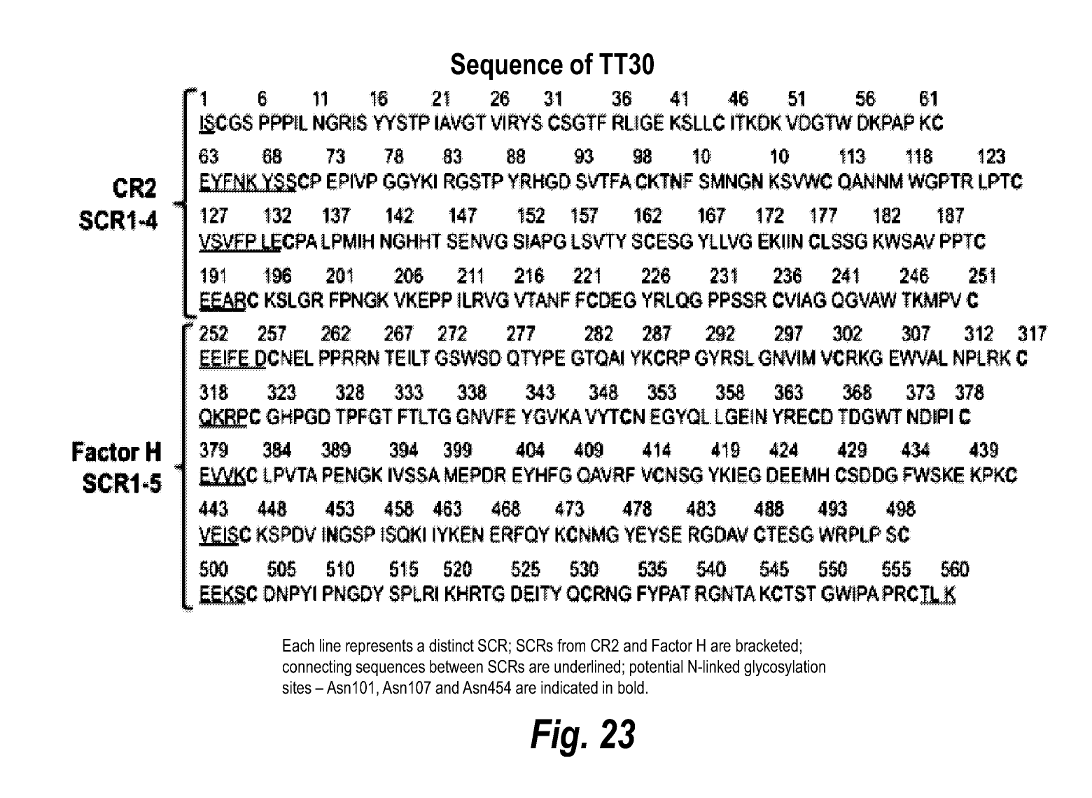

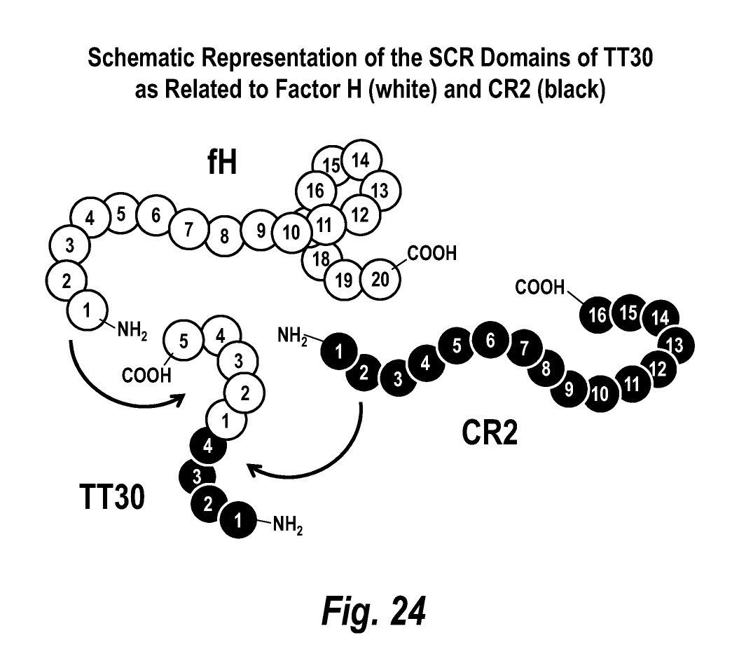

In one embodiment, the complement inhibitor is a fusion protein comprising a complement receptor 2 (CR2) fragment linked to a complement inhibitory domain of complement factor H (CFH). In another embodiment, the complement inhibitor is a human CR2-FH fusion protein comprising SEQ ID NO:3. In a particular embodiment the complement inhibitor is TT30 (also known as ALXN1102). FIGS. 23-24 depict the sequence of TT30 and distinguish the CR2 and Factor H portions.

### Factor H Molecule Capable of Inhibiting Alternative Complement Activation

Factor H is a known inhibitor of the alternative complement pathway. The present invention provides a factor H molecule, compositions (such as pharmaceutical compositions) comprising a factor H molecule, and methods of improving graft survival, decreasing ischemia-reperfusion injury or other endogenous hyperacute, acute, or chronic rejections to the transplanted organ. Factor H molecules in this application include wild-type, mutated forms, or other modified forms of factor H. In one embodiment, the factor H molecule is a factor H-fusion protein. In one embodiment, the factor H fusion protein comprises factor H fused to a targeting moiety to the C3b activation site on the cell or pathogen surface. In a particular embodiment, such a fusion protein comprises a complement receptor 2 (CR2)-factor H fusion protein.

The CR2-FH molecule comprises a CR2 portion and a FH portion. The CR2 portion is responsible for targeted delivery of the molecule to the sites of complement activation, and the FH portion is responsible for specifically inhibiting complement activation of the alternative pathway. Preliminary studies have shown that a CR2-FH molecule, specifically, a CR2-FH fusion protein containing the first four N-terminal SCR domains of the CR2 protein and the first five N-terminal SCR domains the factor H protein (also referred as TT30), has both targeting activity and complement inhibitory activity in vitro. This molecule is significantly more effective than a factor H molecule lacking the CR2 portion, suggesting that targeting FH to complement activation sites will be an effective therapeutic tool in treating diseases in which the alternative complement pathway is implicated, such as macular degeneration (for example age-related macular degeneration). This observation is surprising because of the relatively high concentration of FH in the plasma and the long-held belief that cells which are in direct contact with plasma are already completely covered with FH. Jozsi et al., Histopathol. (2004) 19:251-258.

“CR2-FH molecule” used herein refers to a non-naturally-occurring molecule comprising a CR2 or a fragment thereof (the “CR2 portion”) and a FH or a fragment thereof (the “FH portion”). The CR2 portion is capable of binding to one or more natural ligands of CR2 and is thus responsible for targeted delivery of the molecule to the sites of complement activation. The FH portion is responsible for specifically inhibiting complement activation of the alternative complement pathway. The CR2 portion and the FH portion of the CR2-FH molecule can be linked together by any methods known in the art, as long as the desired functionalities of the two portions are maintained. The CR2 and/or the FH portion may comprise CR2 or FH proteins originated from mammals or other species, their homologs, orthologs, paralogs, optionally with any modifications known in the art not interfering with, or actually improving, its function. The mammals or other species may include, at least, human, mouse, rat, monkey, sheep, dog, cat, pig, rabbit, cow, goat, horse, camelid, chicken, or other animals known in the art and/or used in practice.

The CR2-FH molecule described herein thus generally has the dual functions of binding to a CR2 ligand and inhibiting complement activation of the alternative pathway. “CR2 ligand” refers to any molecule that binds to a naturally-occurring CR2 protein, which include, but are not limited to, C3b, iC3b, C3dg, C3d, and cell-bound fragments of C3b that bind to the two N-terminal SCR domains of CR2. The CR2-FH molecule may, for example, bind to a CR2 ligand with a binding affinity that is about any of 10%, 20%, 30%, 40%, 50%, 60%, 70%, 80%, 90%, or 100% of the CR2 protein. Binding affinity can be determined by any method known in the art, including for example, surface plasmon resonance, calorimetry titration, ELISA, and flow cytometry. In some embodiments, the CR2-FH molecule has one or more of the following properties of CR2: (1) binding to C3d, (2) binding to iC3b, (3) binding to C3dg, (4) binding to C3d, and (5) binding to cell-bound fragment(s) of C3b that bind to the two N-terminal SCR domains of CR2.

The CR2-FH molecule described herein is generally capable of inhibiting complement activation of the alternative pathway. The CR2-FH molecule may be a more potent complement inhibitor than the naturally-occurring FH protein. For example, in some embodiments, the CR2-FH molecule has a complement inhibitory activity that is about any of 1.5, 2, 2.5, 3, 3.5, 4, 5, 6, 7, 8, 9, 10, 12, 14, 16, 18, 20, 25, 30, 40, or more fold of that of the FH protein. In some embodiments, the CR2-FH molecule has an EC50 of less than about any of 100 nM, 90 nM, 80 nM, 70 nM, 60 nM, 50 nM, 40 nM, 30 nM, 20 nM, or 10 nM. In some embodiments, the CR2-FH molecule has an EC50 of about 5-60 nM, including for example any of 8-50 nM, 8-20 nM, 10-40 nM, and 20-30 nM. In some embodiments, the CR2-FH molecule has complement inhibitory activity that is about any of 50%, 60%, 70%, 80%, 90%, or 100% of that of the FH protein.

Complement inhibition can be evaluated based on any methods known in the art, including for example, in vitro zymosan assays, assays for lysis of erythrocytes, immune complex activation assays, and mannan activation assays. In some embodiments, the CR2-FH has one or more of the following properties of FH: (1) binding to C-reactive protein (CRP), (2) binding to C3b, (3) binding to heparin, (4) binding to sialic acid, (5) binding to endothelial cell surfaces, (6) binding to cellular integrin receptor, (7) binding to pathogens, (8) C3b co-factor activity, (9) C3b decay-acceleration activity, and (10) inhibiting the alternative complement pathway.

In some embodiments, the CR2-FH molecule is a fusion protein. “Fusion protein” used herein refers to two or more peptides, polypeptides, or proteins operably linked to each other. In some embodiments, the CR2 portion and the FH portion are directly fused to each other. In some embodiments, the CR2 portion and the FH portion are linked by an amino acid linker sequence. Examples of linker sequences are known in the art, and include, for example, (Gly₄Ser), (Gly₄Ser)₂, (Gly₄Ser)₃, (Gly₃Ser)₄, (SerGly₄), (SerGly₄)₂, (SerGly₄)₃, and (SerGly₄)₄. Linking sequences can also comprise “natural” linking sequences found between different domains of complement factors. For example, VSVFPLE, the linking sequence between the first two N-terminal short consensus repeat domains of human CR2, can be used. In some embodiments, the linking sequence between the fourth and the fifth N-terminal short consensus repeat domains of human CR2 (EEIF) is used. The order of CR2 portion and FH portion in the fusion protein can vary. For example, in some embodiments, the C-terminus of the CR2 portion is fused (directly or indirectly) to the N-terminus of the FH portion of the molecule. In some embodiments, the N-terminus of the CR2 portion is fused (directly or indirectly) to the C-terminus of the FH portion of the molecule.

In some embodiments, the CR2-FH molecule is a CR2-FH fusion protein having an amino acid sequence of any of SEQ ID NO:3, SEQ ID NO:21, and SEQ ID NO:23. In some embodiments, the CR2-FH molecule is a fusion protein having an amino acid sequence that is at least about 50%, 60%, 70%, 80%, 90%, 91%, 92%, 93%, 94%, 95%, 96%, 97%, 98%, or 99% identical to that of any of SEQ ID NO:3, SEQ ID NO:21, or SEQ ID NO:23. In some embodiments, the CR2-FH molecule comprises at least about 400, 450, 500, 550, or more contiguous amino acids of any of SEQ ID NO:3, SEQ ID NO:21, and SEQ ID NO:23. In one embodiment, the CR2-FH fusion protein is TT30.

In some embodiments, the CR2-FH molecule is a CR2-FH fusion protein having an amino acid sequence of any of SEQ ID NOs:5-10. In some embodiments, the CR2-FH molecule is a fusion protein having an amino acid sequence that is at least about 50%, 60%, 70%, 80%, 90%, 91%, 92%, 93%, 94%, 95%, 96%, 97%, 98%, or 99% identical to that of any of SEQ ID NOs:5-10. In some embodiments, the CR2-FH molecule comprises at least about 400, 450, 500, 550, or more contiguous amino acids any of SEQ ID NOs:5-10.

In some embodiments, the CR2-FH molecule is encoded by a polynucleotide having nucleic acid sequence of any of SEQ ID NO:4, SEQ ID NO:22, and SEQ ID NO:24. In some embodiments, the CR2-FH molecule is encoded by a polynucleotide having a nucleic acid sequence that is at least about 50%, 60%, 70%, 80%, 90%, 91%, 92%, 93%, 94%, 95%, 96%, 97%, 98%, or 99% identical to that of any of SEQ ID NO:4, SEQ ID NO:22, and SEQ ID NO:24.

In some embodiments, the CR2-FH molecule comprises a CR2 portion and a FH portion linked via a chemical cross-linker. Linking of the two portions can occur on reactive groups located on the two portions. Reactive groups that can be targeted using a crosslinker include primary amines, sulfhydryls, carbonyls, carbohydrates, and carboxylic acids, or active groups that can be added to proteins. Examples of chemical linkers are well known in the art and include, but are not limited to, bismaleimidohexane, maleimidobenzoyl-N-hydroxysuccinimide ester, NHS-Esters-Maleimide Crosslinkers, such as SPDP, carbodiimide, glutaraldehyde, MBS, Sulfo-MBS, SMPB, sulfo-SMPB, GMBS, Sulfo-GMBS, EMCS, Sulfo-EMCS, imidoester crosslinkers, such as DMA, DMP, DMS, DTBP, EDC and DTME.

In some embodiments, the CR2 portion and the FH portion are non-covalently linked. For example, the two portions may be brought together by two interacting bridging proteins (such as biotin and streptavidin), each linked to a CR2 portion or a FH portion.

In some embodiments, the CR2-FH molecule comprises two or more (same or different) CR2 portions described herein. In some embodiments, the CR2-FH molecule comprises two or more (same or different) FH portions described herein. These two or more CR2 (or FH) portions may be tandemly linked (such as fused) to each other. In some embodiments, the CR2-FH molecule (such a CR2-FH fusion protein) comprises a CR2 portion and two or more (such as three, four, five, or more) FH portions. In some embodiments, the CR2-FH molecule (such a CR2-FH fusion protein) comprises a FH portion and two or more (such as three, four, five, or more) CR2 portions. In some embodiments, the CR2-FH molecule (such a CR2-FH fusion protein) comprises two or more CR2 portions and two or more FH portions.

In some embodiments, there is provided an isolated CR2-FH molecule. In some embodiments, the CR2-FH molecules form dimers or multimers.

The CR2 portion and the FH portion in the molecule can be from the same species (such as human or mouse), or from different species.

### CR2 Portion

The CR2 portion described herein comprises a CR2 or a fragment thereof. CR2 is a transmembrane protein expressed predominantly on mature B cells and follicular dendritic cells. CR2 is a member of the C3 binding protein family. Natural ligands for CR2 include, for example, iC3b, C3dg, and C3d, and cell-bound breakdown fragments of C3b that bind to the two N-terminal SCR domains of CR2. Cleavage of C3 results initially in the generation of C3b and the covalent attachment of this C3b to the activating cell surface. The C3b fragment is involved in the generation of enzymatic complexes that amplify the complement cascade. On a cell surface, C3b is rapidly converted to inactive iC3b, particularly when deposited on a host surface containing regulators of complement activation (i.e., most host tissue). Even in absence of membrane-bound complement regulators, substantial levels of iC3b are formed. iC3b is subsequently digested to the membrane-bound fragments C3dg and then C3d by serum proteases, but this process is relatively slow. Thus, the C3 ligands for CR2 are relatively long lived once they are generated and will be present in high concentrations at sites of complement activation. CR2 therefore can serve as a potent targeting vehicle for bringing molecules to the site of complement activation.

CR2 contains an extracellular portion having 15 or 16 repeating units known as short consensus repeats (SCR domains). The SCR domains have a typical framework of highly conserved residues including four cysteines, two prolines, one tryptophane and several other partially-conserved glycines and hydrophobic residues. SEQ ID NO:1 represents the full-length human CR2 protein sequence. Amino acids 1-20 comprise the leader peptide, amino acids 23-82 comprise SCR1, amino acids 91-146 comprise SCR2, amino acids 154-210 comprise SCR3, amino acids 215-271 comprise SCR4. The active site (C3d binding site) is located in SCR1-2 (the first two N-terminal SCR domains). These SCR domains are separated by short sequences of variable length that serve as spacers. The full-length mouse CR2 protein sequence is represented herein by SEQ ID NO:15. The SCR1 and SCR2 domains of the mouse CR2 protein are located with the mouse CR2 amino sequence at positions 14-73 of SEQ ID NO:15 (SCR1) and positions 82-138 of SEQ ID NO:15 (SCR2). Human and mouse CR2 are approximately 66% identical over the full length amino acid sequences represented by SEQ ID NO:1 and SEQ ID NO:15, and approximately 61% identical over the SCR1-SCR2 regions of SEQ ID NO:1 and SEQ ID NO:15. Both mouse and human CR2 bind to C3 (in the C3d region). It is understood that species and strain variations exist for the disclosed peptides, polypeptides, and proteins, and that the CR2 or a fragment thereof described herein encompasses all species and strain variations.

The CR2 portion disclosed herein refers to a polypeptide that contains some or all of the ligand-binding sites of the CR2 protein, and includes, but is not limited to, full-length CR2 proteins (such as human CR2 as shown in SEQ ID NO:1 or mouse CR2 as shown in SEQ ID NO:15), soluble CR2 proteins (such as a CR2 fragment comprising the extracellular domain of CR2), other biologically-active fragments of CR2, a CR2 fragment comprising SCR1 and SCR2, or any homologue of a naturally-occurring CR2 or fragment thereof, as described in detail below. In some embodiments, the CR2 portion has one of the following properties or CR2: (1) binding to C3d, (2) binding to iC3b, (3) binding to C3dg, (4) binding to C3d, and (5) binding to cell-bound fragment(s) of C3b that bind to the two N-terminal SCR domains of CR2.

In some embodiments, the CR2 portion comprises the first two N-terminal SCR domains of CR2. In some embodiments, the CR2 portion comprises the first three N-terminal SCR domains of CR2. In some embodiments, the CR2 portion comprises the first four N-terminal SCR domains of CR2. In some embodiments, the CR2 portion comprises (and in some embodiments consists of or consists essentially of) at least the first two N-terminal SCR domains of CR2, including for example at least any of the first 3, 4, 5, 6, 7, 8, 9, 10, 11, 12, 13, 14, 15, or 16 SCR domains of CR2.

A homologue of a CR2 protein or a fragment thereof includes proteins which differ from a naturally-occurring CR2 (or CR2 fragment) in that at least one or a few amino acids have been deleted (e.g., a truncated version of the protein, such as a peptide or fragment), inserted, inverted, substituted and/or derivatized (e.g., by glycosylation, phosphorylation, acetylation, myristoylation, prenylation, palmitation, amidation and/or addition of glycosylphosphatidyl inositol). In some embodiments, a CR2 homologue has an amino acid sequence that is at least about 70% identical to the amino acid sequence of a naturally-occurring CR2 (e.g., SEQ ID NO:1, or SEQ ID NO:15), for example at least about any of 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of a naturally-occurring CR2 (e.g., SEQ ID NO:1, or SEQ ID NO:15). A CR2 homologue or a fragment thereof preferably retains the ability to bind to a naturally-occurring ligand of CR2 (e.g., C3d or other C3 fragments with CR2-binding ability). For example, the CR2 homologue (or fragment thereof) may have a binding affinity for C3d that is at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of that of CR2 (or a fragment thereof).

In some embodiments, the CR2 portion comprises at least the first two N-terminal SCR domains of a human CR2, such as a CR2 portion having an amino acid sequence containing at least amino acids 23 through 146 of the human CR2 (SEQ ID NO:1). In some embodiments, the CR2 portion comprises at least the first two SCR domains of human CR2 having an amino acid sequence that is at least about any of 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to amino acids 23 through 146 of the human CR2 (SEQ ID NO:1).

In some embodiments, the CR2 portion comprises at least the first four N-terminal SCR domains of a human CR2, such as a CR2 portion having an amino acid sequence containing at least amino acids 23 through 271 of the human CR2 (SEQ ID NO:1). In some embodiments, the CR2 portion comprises at least the first four SCR domains of human CR2 having an amino acid sequence that is at least about any of 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to amino acids 23 through 271 of the human CR2 (SEQ ID NO:1).

An amino acid sequence that is at least about, for example, 95% identical to a reference sequence (such as SEQ ID NO:1) is intended that the amino acid sequence is identical to the reference sequence except that the amino acid sequence may include up to five point alterations per each 100 amino acids of the reference sequence. These up to five point alterations may be deletions, substitutions, additions, and may occur anywhere in the sequence, interspersed either individually among amino acids in the reference sequence or in one or more continuous groups within the reference sequence.

In some embodiments, the CR2 portion comprises part or all of the ligand-binding sites of the CR2 protein. In some embodiments, the CR2 portion further comprises sequences required to maintain the three-dimensional structure of the binding site. Ligand-binding sites of CR2 can be readily determined based on the crystal structures of CR2, such as the human and mouse CR2 crystal structures disclosed in U.S. Patent Application Publication No. 2004/0005538. For example, in some embodiments, the CR2 portion comprises the B strand and B-C loop of SCR2 of CR2. In some embodiments, the CR2 portion comprises a site on strand B and the B-C loop of CR2 SCR comprising the segment G98-G99-Y100-K101-I102-R103-G104-S105-T106-P107-Y108 with respect to SEQ ID NO: 1. In some embodiments, the CR2 portion comprises a site on the B strand of CR2 SCR2 comprising position K119 with respect to SEQ ID NO:1. In some embodiments, the CR2 portion comprises a segment comprising V149-F150-P151-L152, with respect to SEQ ID NO:1. In some embodiments, the CR2 portion comprises a segment of CR2 SCR2 comprising T120-N121-F122. In some embodiments, the CR2-FH molecule has two or more of these sites. For example, in some embodiments, the CR2 portion comprises a portion comprising G98-G99-Y100-K101-I102-R103-G104-S105-T106-P107-Y108 and K119 with respect to SEQ ID NO:1. Other combinations of these sites are also contemplated.

### Factor H Portion

The FH portion of the CR2-FH molecule described herein comprises a FH or a fragment thereof.

Complement factor H (FH) is a single polypeptide chain plasma glycoprotein. The protein is composed of 20 repetitive SCR domains of approximately 60 amino acids, arranged in a continuous fashion like a string of 20 beads. Factor H binds to C3b, accelerates the decay of the alternative pathway C3-convertase (C3Bb), and acts as a cofactor for the proteolytic inactivation of C3b. In the presence of factor H, C3b proteolysis results in the cleavage of C3b. Factor H has at least three distinct binding domains for C3b, which are located within SCR 1-4, SCR 5-8, and SCR 19-20. Each site of factor H binds to a distinct region within the C3b protein: the N-terminal sites bind to native C3b; the second site, located in the middle region of factor H, binds to the C3c fragment and the sited located within SCR19 and 20 binds to the C3d region. In addition, factor H also contains binding sites for heparin, which are located within SCR 7, SCR 5-12, and SCR20 of factor H and overlap with that of the C3b-binding site. Structural and functional analyses have shown that the domains for the complement inhibitory activity of FH are located within the first four N-terminal SCR domains.

SEQ ID NO:2 represents the full-length human FH protein sequence. Amino acids 1-18 correspond to the leader peptide, amino acids 21-80 correspond to SCR1, amino acids 85-141 correspond to SCR2, amino acids 146-205 correspond to SCR3, amino acids 210-262 correspond to SCR4, amino acids 267-320 correspond to SCR5. The full-length mouse FH protein sequence is represented herein by SEQ ID NO:16. The SCR1 and SCR2 domains of the mouse FH protein are located with the mouse FH amino sequence at positions 21-27 of SEQ ID NO:16 (SCR1) and positions 82-138 of SEQ ID NO:16 (SCR2). Human and mouse FH are approximately 61% identical over the full length amino acid sequences represented by SEQ ID NO:2 and SEQ ID NO:16. It is understood that species and strain variations exist for the disclosed peptides, polypeptides, and proteins, and that the FH or a fragment thereof encompasses all species and strain variations.

The FH portion described herein refers to any portion of a FH protein having some or all the complement inhibitory activity of the FH protein, and includes, but is not limited to, full-length FH proteins, biologically-active fragments of FH proteins, a FH fragment comprising SCR1-4, or any homologue of a naturally-occurring FH or fragment thereof, as described in detail below. In some embodiments, the FH portion has one or more of the following properties: (1) binding to C-reactive protein (CRP), (2) binding to C3b, (3) binding to heparin, (4) binding to sialic acid, (5) binding to endothelial cell surfaces, (6) binding to cellular integrin receptor, (7) binding to pathogens, (8) C3b co-factor activity, (9) C3b decay-acceleration activity, and (10) inhibiting the alternative complement pathway.

In some embodiments, the FH portion comprises the first four N-terminal SCR domains of FH. In some embodiments, the construct comprises the first five N-terminal SCR domains of FH. In some embodiments, the construct comprises the first six N-terminal SCR domains of FH. In some embodiments, the FH portion comprises (and in some embodiments consists of or consisting essentially of) at least the first four N-terminal SCR domains of FH, including for example, at least any of the first 5, 6, 7, 8, 9, 10, 11, 12, 13, 14, or more N-terminal SCR domains of FH.

In some embodiments, the FH is a wild type FH. In some embodiments, the FH is a protective variant of FH.

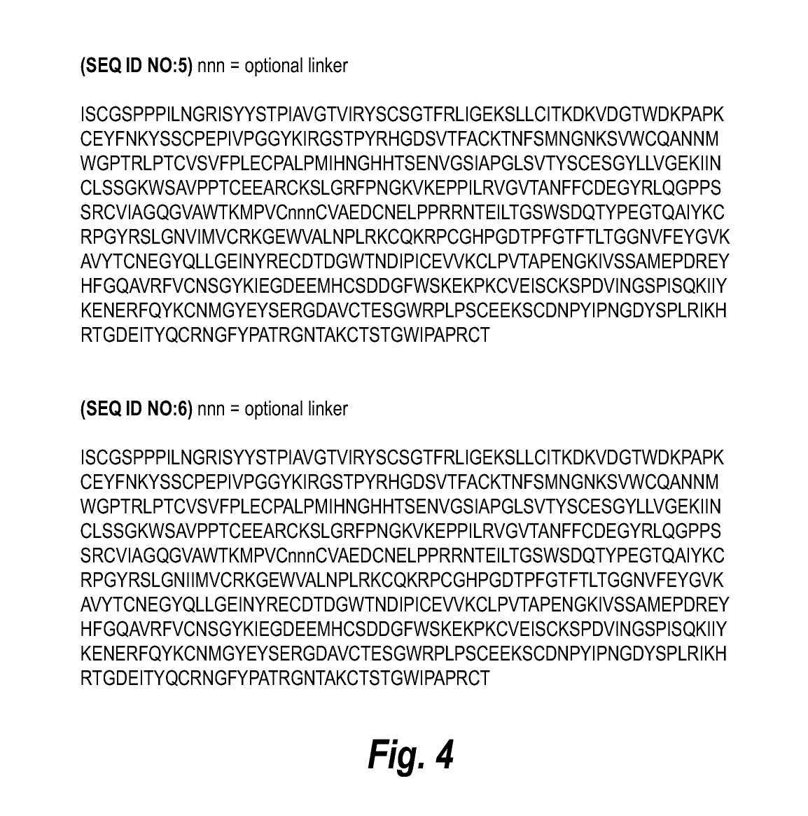

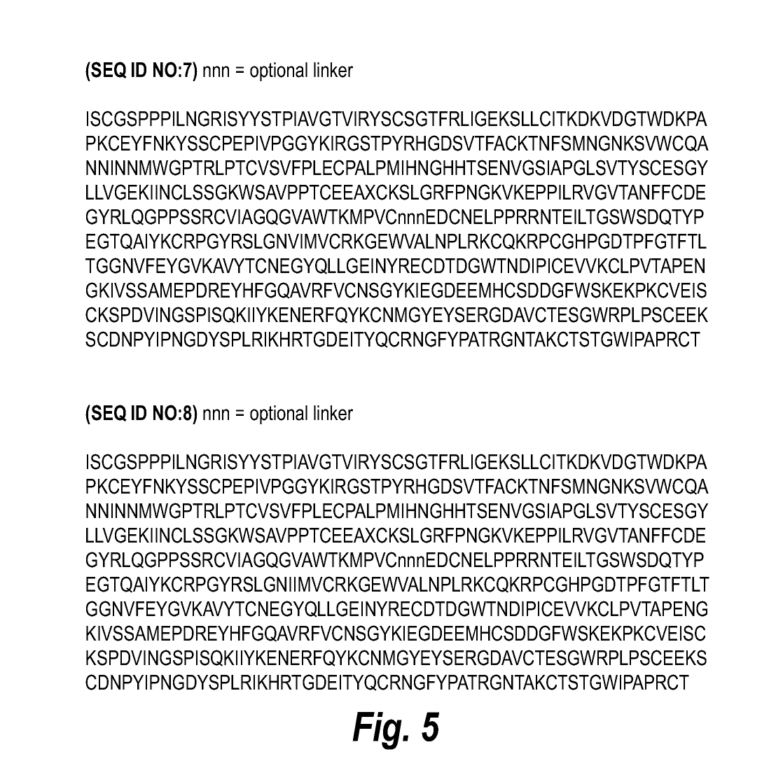

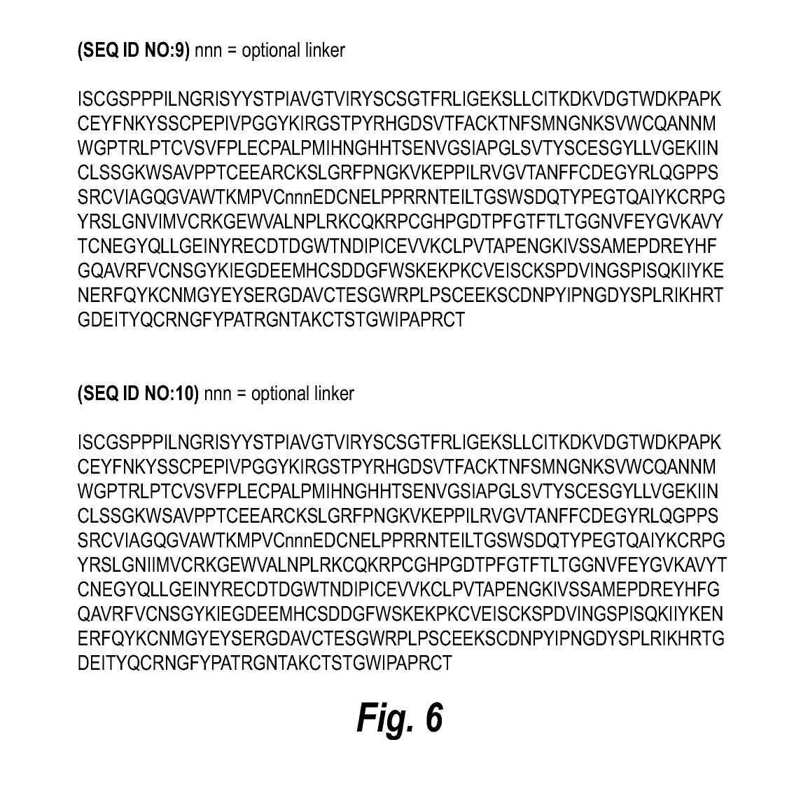

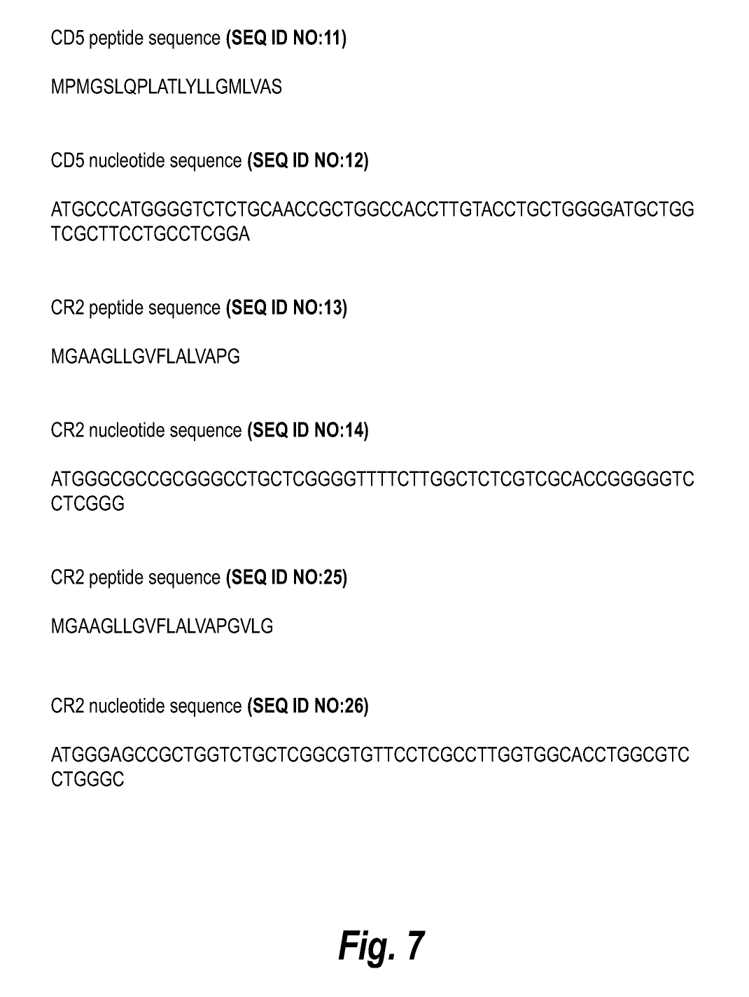

In some embodiments, the FH portion lacks a heparin-binding site. This can be achieved, for example, by mutation of the heparin-binding site on FH, or by selecting FH fragments that do not contain a heparin-binding site. In some embodiments, the FH portion comprises a FH or a fragment thereof having a polymorphism that is protective to age-related macular degeneration. Hageman et al., Proc. Natl. Acad. Sci. USA 102(20):7227. One example of a CR2-FH molecule comprising such a sequence is provided in FIG. 4 (SEQ ID NO:6).

A homologue of a FH protein or a fragment thereof includes proteins which differ from a naturally-occurring FH (or FH fragment) in that at least one or a few, but not limited to one or a few, amino acids have been deleted (e.g., a truncated version of the protein, such as a peptide or fragment), inserted, inverted, substituted and/or derivatized (e.g., by glycosylation, phosphorylation, acetylation, myristoylation, prenylation, palmitation, amidation and/or addition of glycosylphosphatidyl inositol). For example, a FH homologue may have an amino acid sequence that is at least about 70% identical to the amino acid sequence of a naturally-occurring FH (e.g., SEQ ID NO:2, or SEQ ID NO:16), for example at least about any of 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of a naturally-occurring FH (e.g., SEQ ID NO:2, or SEQ ID NO:16). In some embodiment, a homologue of FH (or a fragment thereof) retains all the complement inhibition activity of FH (or a fragment thereof). In some embodiments, the homologue of FH (or a fragment thereof) retains at least about 50%, for example, at least about any of 60%, 70%, 80%, 90%, or 95% of the complement inhibition activity of FH (or a fragment thereof).

In some embodiments, the FH portion comprises at least the first four N-terminal SCR domains of a human FH, such as a FH portion having an amino acid sequence containing at least amino acids 21 through 262 of the human FH (SEQ ID NO:2). In some embodiments, the FH portion comprises at least the first four N-terminal SCR domains of human FH having an amino acid sequence that is at least about any of 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to amino acids 21 through 262 of the human FH (SEQ ID NO:2).

In some embodiments, the FH portion comprises at least the first five N-terminal SCR domains of a human FH, such as a FH portion having an amino acid sequence containing at least amino acids 21 through 320 of the human FH (SEQ ID NO:2). In some embodiments, the FH portion comprises at least the first five N-terminal SCR domains of human FH having an amino acid sequence that is at least about any of 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to amino acids 21 through 320 of the human FH (SEQ ID NO:2).

In some embodiments, the FH portion comprises a full length or a fragment of factor-H like 1 molecule (FHL-1), a protein encoded by an alternatively spliced transcript of the factor H gene. The mature FHL-1 contains 431 amino acids. The first 427 amino acids organize seven SCR domains and are identical to the N-terminal SCR domains of FH. The remaining four amino acid residues Ser-Phe-Thr-Leu (SFTL) at the C-terminus are specific to FHL-1. FHL-1 has been characterized functionally and shown to have factor H complement regulatory activity. The term “FH portion” also encompasses full length or fragments of factor H related molecules, including, but are not limited to, proteins encoded by the FHR1, FHR2, FHR3, FHR4, FHR5 genes. These factor H related proteins are disclosed, for example, in de Cordoba et al., Molecular Immunology 2004, 41:355-367.

### Variants of CR2-FH Molecules

Also encompassed in the methods and compositions of the invention are variants of the CR2-FH molecules (such as the CR2-FH fusion proteins). A variant of the CR2-FH molecule described herein may be: (i) one in which one or more of the amino acid residues of the CR2 portion and/or the FH portion are substituted with a conserved or non-conserved amino acid residue (preferably a conserved amino acid residue) and such substituted amino acid residue may or may not be one encoded by the genetic code; or (ii) one in which one or more of the amino acid residues in the CR2 portion and/or FH portion includes a substituent group, or (iii) one in which the CR2-FH molecule (such as the CR2-FH fusion protein) is fused with another compound, such as a compound to increase the half-life of the CR2-FH molecule (for example, polyethylene glycol), or (iv) one in which additional amino acids are fused to the CR2-FH molecule (such as the CR2-FH fusion protein), such as a leader or secretory sequence or a sequence which is employed for purification of the CR2-FH molecule (such as the CR2-FH fusion protein), or (v) one in which the CR2-FH molecule (such as the CR2-FH fusion protein) is fused with a larger polypeptide, i.e., human albumin, an antibody or Fc, for increased duration of effect. Such variants are deemed to be within the scope of those skilled in the art from the teachings herein.

In some embodiments, the variant of the CR2-FH molecule contains conservative amino acid substitutions (defined further below) made at one or more predicted, preferably nonessential, amino acid residues. A “nonessential” amino acid residue is a residue that can be altered from the wild-type sequence of a protein without altering the biological activity, whereas an “essential” amino acid residue is required for biological activity. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Amino acid substitutions in the CR2 or FH portions of the CR2-FH molecule can be introduced to improve the functionality of the molecule. For example, amino acid substitutions can be introduced into the CR2 portion of the molecule to increase binding affinity of the CR2 portion to its ligand(s), increase binding specificity of the CR2 portion to its ligand(s), improve targeting of the CR2-FH molecule to desired sites, increase dimerization or multimerization of CR2-FH molecules, and improve pharmacokinetics of the CR2-FH molecule. Similarly, amino acid substitutions can be introduced into the FH portion of the molecule to increase the functionality of the CR2-FH molecule and improve pharmacokinetics of the CR2-FH molecule.

In some embodiments, the CR2-FH molecule (such as the CR2-FH fusion protein) is fused with another compound, such as a compound to increase the half-life of the polypeptide and/or to reduce potential immunogenicity of the polypeptide (for example, polyethylene glycol, “PEG”). The PEG can be used to impart water solubility, size, slow rate of kidney clearance, and reduced immunogenicity to the fusion protein. See e.g., U.S. Pat. No. 6,214,966. In the case of PEGylations, the fusion of the CR2-FH molecule (such as the CR2-FH fusion protein) to PEG can be accomplished by any means known to one skilled in the art. For example, PEGylation can be accomplished by first introducing a cysteine mutation into the CR2-FH fusion protein, followed by site-specific derivatization with PEG-maleimide. The cysteine can be added to the C-terminus of the CR2-FH fusion protein. See, e.g., Tsutsumi et al. (2000) Proc. Natl. Acad. Sci. USA 97(15):8548-8553. Another modification which can be made to the CR2-FH molecule (such as the CR2-FH fusion protein) involves biotinylation. In certain instances, it may be useful to have the CR2-FH molecule (such as the CR2-FH fusion protein) biotinylated so that it can readily react with streptavidin. Methods for biotinylation of proteins are well known in the art. Additionally, chondroitin sulfate can be linked with the CR2-FH molecule (such as the CR2-FH fusion protein).

In some embodiments, the CR2-FH molecule is fused to another targeting molecule or targeting moiety which further increases the targeting efficiency of the CR2-FH molecule. For example, the CR2-FH molecule can be fused to a ligand (such as an amino acid sequence) that has the capability to bind or otherwise attach to an endothelial cell of a blood vessel (referred to as “vascular endothelial targeting amino acid ligand”). Exemplary vascular endothelial targeting ligands include, but are not limited to, VEGF, FGF, integrin, fibronectin, I-CAM, PDGF, or an antibody to a molecule expressed on the surface of a vascular endothelial cell.

In some embodiments, the CR2-FH molecule is conjugated (such as fused) to a ligand for intercellular adhesion molecules. For example, the CR2-FH molecule can be conjugated to one or more carbohydrate moieties that bind to an intercellular adhesion molecule. The carbohydrate moiety facilitates localization of the CR2-FH molecule to the site of injury. The carbohydrate moiety can be attached to the CR2-FH molecule by means of an extracellular event such as a chemical or enzymatic attachment, or can be the result of an intracellular processing event achieved by the expression of appropriate enzymes. In some embodiments, the carbohydrate moiety binds to a particular class of adhesion molecules such as integrins or selectins, including E-selectin, L-selectin or P-selectin. In some embodiments, the carbohydrate moiety comprises an N-linked carbohydrate, for example the complex type, including fucosylated and sialylated carbohydrates. In some embodiments, the carbohydrate moiety is related to the Lewis X antigen, for example the sialylated Lewis X antigen. For further descriptions for the CR2-FH fusion protein please see WO 2007/149567, which is incorporated herein by reference in its entirety.

### Immunosuppressive Agents

The numerous drugs utilized to delay graft rejection (i.e., to prolong their survival) work in a variety of ways. Immunosuppressive agents are widely used. See Stepkowski, 2000, for a review of the mechanism of action of several immunosuppressive drugs. Cyclosporin A is one of the most widely used immunosuppressive drugs for inhibiting graft rejection. It is an inhibitor of interleukin-2 or IL-2 (it prevents mRNA transcription of interleukin-2). More directly, cyclosporin inhibits calcineurin activation that normally occurs upon T cell receptor stimulation. Calcineurin dephosphorylates NFAT (nuclear factor of activated T cells) enabling it to enter the nucleus and bind to interleukin-2 promoter. By blocking this process, cyclosporin A inhibits the activation of the CD4⁺ T cells and the resulting cascade of events which would otherwise occur. Tacrolimus is another immunosuppressant that acts by inhibiting the production of interleukin-2.

Rapamycin (Sirolimus), SDZ RAD, and interleukin-2 receptor blockers are drugs that inhibit the action of interleukin-2 and therefore prevent the cascade of events described above.

Inhibitors of purine or pyrimidine biosynthesis are also used to inhibit graft rejection. These prevent DNA synthesis and thereby inhibit cell division including the ability of T cells to divide. The result is the inhibition of T cell activity by preventing the formation of new T cells. Inhibitors of purine synthesis include azathioprine, methotrexate, mycophenolate mofetil (MMF) and mizoribine (bredinin). Inhibitors of pyrimidine synthesis include brequinar sodium, leflunomide and teriflunomide. Cyclophosphamide is an inhibitor of both purine and pyrimidine synthesis.

Yet another method for inhibiting T cell activation is to treat the recipient with antibodies to T cells. OKT3 is a murine monoclonal antibody against CD3, which is part of the T cell receptor. This antibody inhibits the T cell receptor and suppresses T cell activation.

Numerous other drugs and methods for delaying allotransplant rejection are known to and used by those of skill in the art. One approach has been to deplete T cells, e.g., by irradiation. This has often been used in bone marrow transplants, especially if there is a partial mismatch of major HLA. Administration to the recipient of an inhibitor (blocker) of the CD40 ligand-CD40 interaction and/or a blocker of the CD28-B7 interaction has been used (U.S. Pat. No. 6,280,957). Published PCT patent application WO 01/37860 teaches the administration of an anti-CD3 monoclonal antibody and IL-5 to inhibit the Th1 immune response. Published PCT patent application WO 00/27421 teaches a method for prophylaxis or treatment of corneal transplant rejection by administering a tumor necrosis factor-α antagonist. Glotz et al. (2002) show that administration of intravenous immunoglobulins (IVIg) can induce a profound and sustained decrease in the titers of anti-HLA antibodies thereby allowing a transplant of an HLA-mismatched organ. Similar protocols have included plasma exchanges (Taube et al., 1984) or immunoadsorption techniques coupled to immunosuppressive agents (Hiesse et al., 1992) or a combination of these (Montgomery et al., 2000). Changelian et al. (2003) teach a model in which immunosuppression is caused by an oral inhibitor of Janus kinase 3 (JAK3) which is an enzyme necessary for the proper signaling of cytokine receptors which use the common gamma chain (γc) (Interleukins-2, -4, -7, -9, -15, -21), the result being an inhibition of T cell activation. Antisense nucleic acids against ICAM-1 have been used alone or in combination with a monoclonal antibody specific for leukocyte-function associated antigen 1 (LFA-1) in a study of heart allograft transplantation (Stepkowski, 2000). Similarly, an anti-ICAM-1 antibody has been used in combination with anti-LFA-1 antibody to treat heart allografts (Stepkowski, 2000). Antisense oligonucleotides have additionally been used in conjunction with cyclosporin in rat heart or kidney allograft models, resulting in a synergistic effect to prolong the survival of the grafts (Stepkowski, 2000). Chronic transplant rejection has been treated by administering an antagonist of TGF-β which is a cytokine involved in differentiation, proliferation and apoptosis (U.S. Patent Application Publication US 2003/0180301).

One or more of the immunosuppressive drugs described above can be used in the methods of the present invention.

### Methods and Uses

The methods disclosed herein are used to prolong graft survival of an organ that is transplanted from a donor to a recipient. The methods disclosed herein are also used to prevent or attenuate rejection of a transplanted organ, as well as to treat, decrease, or alleviate ischemia-reperfusion injury (IRI) in the recipient of the transplantation. The methods generally include administering an inhibitor of complement activity, optionally in combination with one or more immunosuppressants and/or one or more additional complement inhibitors.

Also provided are methods to prolong survival of an organ that is transplanted from a donor mammal to a recipient mammal, as well as methods to prevent or attenuate rejection (e.g., hyperacute rejection, antibody-mediated rejection, or chronic rejection) of a transplanted organ in a recipient mammal, which involve administering a complement inhibitor to the organ prior to transplantation, wherein the complement inhibitor is particular inhibitor (e.g., TT30 or a single chain anti-C5 antibody, such as pexelizumab or a single chain version of eculizumab) or has a maximum molecular weight of 70 kDa and/or a half-life of less than 10 days.

The methods described herein can be used in different organ transplant scenarios, e.g., for autologous graft or autograft, isograft or syngeneic graft, allogeneic graft or allograft, and xenogeneic graft or xenograft. The methods described herein may be effective to treat hyperacute rejection, acute rejection, delayed graft function, or chronic rejection. In a particular embodiment, a complement inhibitor is not administered to the organ recipient after transplantation.

The complement inhibitor is administered to the organ prior to transplantation (e.g., after removal of the organ from a donor mammal and before transplant of the organ into a recipient mammal). In one embodiment, the complement inhibitor is administered at an organ procurement center. In another embodiment, the complement inhibitor is administered immediately prior to transplantation, e.g., in a “back table” procedure within hours or minutes prior to translation. In one embodiment, complement inhibitor is administered after harvest or removal from the donor mammal, but prior to preservation of the organ. In another embodiment, the complement inhibitor is administered to the organ during preservation. In another embodiment, the complement inhibitor is administered after preservation, but prior to transplantation. In other embodiments, the complement inhibitor is administered in multiple stages as listed above. Further, any of the administrations can be repeated multiple times within a particular time frame. For instance, the administration can involve two or more perfusions or soakings. In another embodiment, a single complement inhibitor can be administered, two or more complement inhibitors can be administered, or a plurality of complement inhibitors can be administered.

The complement inhibitor can be administered to the organ by any suitable technique. In one embodiment, the complement inhibitor is administered to the organ by perfusing the organ with a solution containing the complement inhibitor. In another embodiment, the organ is bathed in a solution containing the complement inhibitor. In one embodiment, the organ is perfused with or soaked in a solution containing the complement inhibitor for 0.5 hours to 60 hours or for 1 hour to 30 hours (e.g., for 30 minutes, 35 minutes, 40 minutes, 45 minutes, 50 minutes, 55 minutes, 1 hour, 1.5 hours, 2 hours, 2.5 hours, 3 hours, 3.5 hours, 4 hours, 4.5 hours, 5 hours, 5.5 hours, 6 hours, 6.5 hours, 7 hours, 7.5 hours, 8 hours, 8.5 hours, 9 hours, 9.5 hours, 10 hours, 10.5 hours, 11 hours, 11.5 hours, 12 hours, 12.5 hours, 13 hours, 13.5 hours, 14 hours, 14.5 hours, 15 hours, 15.5 hours, 16 hours, 16.5 hours, 17 hours, 17.5 hours, 18 hours, 18.5 hours, 19 hours, 19.5 hours, 20 hours, 21 hours, 22 hours, 23 hours, 24 hours, 25 hours, 26 hours, 27 hours, 28 hours, 29 hours, or 30 hours).

In one embodiment, the recipient mammal is not vaccinated (e.g., against Neisseria meningitides) prior to transplantation. In another embodiment, the recipient is not treated with a complement inhibitor after transplantation.

In some embodiments, the amount of CR2-FH present in an organ preservation solution is from about 10 μg to about 500 mg per liter, including for example any of about 10 μg to about 50 μg, about 50 μg to about 100 μg, about 100 μg to about 200 μg, about 200 μg to about 300 μg, about 300 μg to about 500 μg, about 500 μg to about 1 mg, about 1 mg to about 10 mg, about 10 mg to about 50 mg, about 50 mg to about 100 mg, about 100 mg to about 200 mg, about 200 mg to about 300 mg, about 300 mg to about 400 mg, or about 400 mg to about 500 mg per liter. In some embodiments, the amount of CR2-FH (TT30) comprises about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, 8000, 8500, 9000, 9500, 10000, 15000, 20000, 30000, 40000, 50000, 60000, 70000, 80000, 90000, 100000, or above, μg/mL. In some embodiments, the amount of CR2-FH (TT30) comprises about 130 μg/mL.

The CR2-FH compositions can be used alone or in combination with other molecules known to have a beneficial effect, including molecules capable of tissue repair and regeneration and/or inhibiting inflammation. Examples of useful cofactors include anti-VEGF agents (such as an antibody against VEGF), basic fibroblast growth factor (bFGF), ciliary neurotrophic factor (CNTF), axokine (a mutein of CNTF), leukemia inhibitory factor (LIF), neutrotrophin 3 (NT-3), neurotrophin-4 (NT-4), nerve growth factor (NGF), insulin-like growth factor II, prostaglandin E2, 30 kD survival factor, taurine, and vitamin A. Other useful cofactors include symptom-alleviating cofactors, including antiseptics, antibiotics, antiviral and antifungal agents and analgesics and anesthetics.

A “lyoprotectant” is a molecule which, when combined with a drug of interest (e.g., antibody or antigen-binding fragment thereof or a factor H fusion protein), significantly prevents or reduces chemical and/or physical instability of the drug (e.g., antibody or antigen-binding fragment thereof) upon lyophilization and subsequent storage. Exemplary lyoprotectants include sugars, such as sucrose or trehalose; an amino acid such as monosodium glutamate or histidine; a methylamine such as betaine; a lyotropic salt such as magnesium sulfate; a polyol, such as trihydric or higher sugar alcohols, e.g. glycerin, erythritol, glycerol, arabitol, xylitol, sorbitol, and mannitol; propylene glycol; polyethylene glycol; Pluronics; and combinations thereof. The preferred lyoprotectant is a non-reducing sugar, such as trehalose or sucrose. The methods and compositions described herein can include the use or addition of a lyoprotectant.

The lyoprotectant is added to the drug formulation in a “lyoprotecting amount” which means that, following lyophilization of the drug (e.g., antibody or antigen-binding fragment thereof or a factor H fusion protein) in the presence of the lyoprotecting amount of the lyoprotectant, the drug (e.g., antibody or antigen-binding fragment thereof, or a factor H fusion protein) essentially retains its physical and chemical stability and integrity upon lyophilization and storage.

The present methods and uses are described with reference to the following Examples, which are offered by way of illustration and are not intended to limit the disclosure in any manner. Standard techniques well known in the art or the techniques specifically described below are utilized. The following abbreviations are used herein: ABMR, antibody-mediated rejection; ACHR, accelerated humoral rejection; ACR, acute cellular rejection; AVR, acute vascular rejection; CsA, cyclosporin; CyP, cyclophosphamide; HAR, hyperacute rejection; MCP-1, monocyte chemotactic protein 1; MST, mean survival time; POD, postoperative day.

### Example 1

### Methods

### Animals and Immunosuppressive Drugs

Male adult C3H (H-2^{k}) mice and BALB/c (H-2^{d}) mice (Jackson Labs, Bar Harbor, Me.) weighing 25-30 g were chosen as donors and recipients, respectively. In the groups receiving immunosuppression, the recipients were injected with CsA (15 mg/kg/day, s.c., daily from day 0 to endpoint rejection or until day 100), or with CyP (40 mg/kg/day, i.v., on day 0 and 1), or with anti-C5 mAb (clone BB5.1, Alexion Pharmaceuticals Inc.

### Standard Hemolysis Assay Using Chicken Cells

Blood cell hemolysis assays can be carried out in many ways as common knowledge known in the art, for example, in Wang et al. (2007) Inhibition of Terminal Complement Components in Presensitized Transplant Recipients Prevents Antibody-Mediated Rejection Leading to Long-Term Graft Survival and Accommodation. The Journal of Immunology, 179: 4451-4463. An exemplary method was given as below:

### Reagents:

GVBS buffer (containing Mg²⁺ and Ca²⁺) was obtained from Complement Technology, Inc. (Tyler, Tex.; cat# B100). Chicken erythrocytes were obtained from Lampire (Pipersville, Pa.; cat #7201403) in Alsever's solution. Anti-chicken IgG (sensitizing antibody) was obtained from Intercell Technologies (Hopewell, N.J.). Normal mouse and normal human serum were obtained from Bioreclamation (Baltimore, Md.).

### Methods:

The test sample (i.e., mAb, Fab, fusion protein) and serum (i.e., human serum) were individually titrated in GVBS to a concentration twice the desired final concentration. Fifty microliters of such sample solution were loaded to each well of a 96-well U bottom Nunc™ plate (Thermo Scientific, Waltham, Mass.) by titrating your sample (i.e. mAb) in GVBS such that you have 50 μL/well of a solution of TWICE the desired final concentration. Fifty microliters of such serum solution were added to each sample well. This will give a total volume of 100 μL with 1× of each component (serum and sample). Assay controls were added to other wells in parallel, which include: 100 μL GVBS as negative control, 100 μL GVBS plus 2 μL NP40 as positive control, serum without inhibitors (containing 10 mM EDTA) as reference blank/background, and serum without inhibitors as positive control for 100% serum lysis.

Four hundred microliters of chicken blood cells (around 1×10⁹ cells/ml) were washed with 1 mL GVBS and collected by centrifugation at around 3,000 rpm for 1 minute at 4° C. Cells were resuspended and washed for four times. After the final wash, cell pellet was resuspended to about 400 μL by adding about 300 μL GVBS. From the suspension, 210 μL of chicken blood cells were mixed with GVBS in a final volume of 6 mL to reach a final concentration of 5×10⁷ cells/ml. Six microliters of anti-chicken IgG (0.1% v/v) were added to the solution and the resulting mixture was inverted to mix and incubate on ice for 15 minutes. Then the mixture was spun at 3,000 rpm at 4° C. for 1 minute. The resulting supernatant was removed by aspiration and the pellet was resuspended in GVBS to a volume of 6 mL. The suspension was spun again and the resulting pellet was resuspended to a final volume of 3.6 mL. Among them 30 μL of cells (about 2.5×10⁶ cells) were added to each well of the sample plate containing the test sample (or controls). Each well was covered with adhesive plate sealer before tapping to mix and incubate at 37° C. for 30 minutes. After spinning the plate, 85 μL of supernatant were transferred, without disturbing the cell pellet, to a 96-well Flat bottom Nunc™ plate (Thermo Scientific) for reading OD at 415 nm. The % lysis was calculated by dividing the difference of OD readings between test sample and reference blank by the reading difference between 100% serum lysis control and reference blank, i.e., (Sample A415−reference blank 415)/(100% serum max 415−reference blank 415)

### Rabbit Red Cell Assay for Alternative Pathway Activity

1. Cell Prep Methods

The concentration of red blood cells in rabbit blood (Lampire, cat #7206403, in Alsever's solution) was determined to be approximately 10⁹ cells/mL. The determination method involves reading OD at 412 nm for the mixture of 100 μL rabbit blood and 2.9 mL water. The correlation between the OD reading and the cell concentration is that an OD 412 of 0.29=1×10⁸ cells/mL. Four hundred microliters of rabbit blood were washed with 1 mL GVBS (containing 2 mM MgCl₂ and 10 mM EGTA) for four times. After final wash, the rabbit red cell pellet was resuspended back to 400 μL by adding 300 μL GVBS. Among them, 50 μL of suspended cells was transferred out for dilution to 1 mL with GVBS. Thirty microliters of such diluted solution were mixed with 100 μL prepared sample in well of 96 well plate (this gives ˜1.5×10⁶ cells/well). The plate was incubated at 37° C. for 30 minutes before 85 μL supernatant of each well were transferred to a 96-well Flat bottom Nunc™ plate (Thermo Scientific) for reading OD at 415 nm.

### Perfusion and Preservation of the Donor Organ

1. 1^{st} perfusion of donor organ with UW solution right after donor organ harvested;
2. donor organ preservation in UW solution at 4° C. for 28 hours;
3. 2^{nd} perfusion of donor organ at 30-45 minutes prior transplant (the solution for recipient only treatment groups (Group 1 to 4, 6-7) was UW; the solution for donor organ and recipient treatment group (Group 5) was UW containing 130 μg/ml hTT30 without further flushing out;
4. After 2^{nd} perfusion, the donor organs were preserved in an ice-surrounded container with the same solution as that for 2^{nd} perfusion for 30-45 minutes prior to transplantation.
   The conditions for the above donor organ perfusions were:
1. Total volume: 2.5 ml
2. Time: 20-30 second
3. Syringe size: 3cc
4. Operate manually, pressure: low

### Example 2

### TT30 Effectively Inhibits Complement Alternative Pathway in Rat Serum

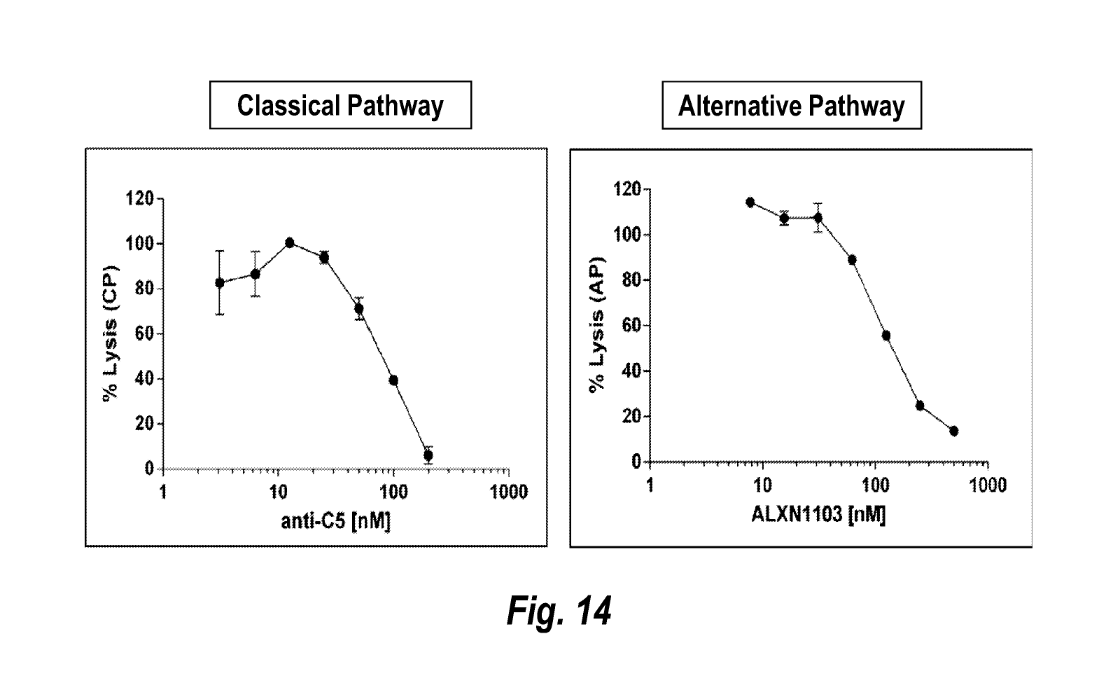

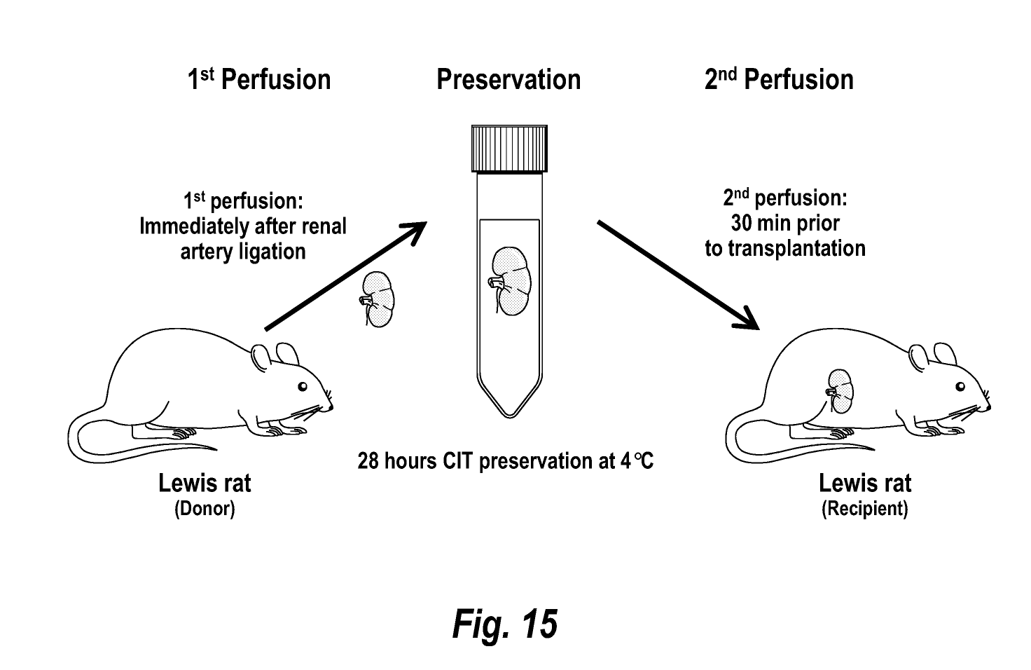

Anti-C5 monoclonal antibody 18A10 (an anti-rat C5 antibody) and human TT30 (CR2-FH) were incubated with healthy rat serum to evaluate the capacity to inhibit the classical (CCP) and alternative (CAP) complement pathways, respectively. The potency of anti-C5 monoclonal antibody was measured as inhibition of CCP by using sensitized chicken red blood cells (RBCs) and for lysis in 50% Lewis rat serum at 37° C. for 30 minutes. The potency of hTT30 was measured as inhibition of CAP by using rabbit RBCs for lysis in 20% Lewis rat serum at 37° C. for 30 minutes. hTT30 was added into rat serum at different concentration (up to 500 nM) alone or in the presence of excess anti-huCR2 monoclonal antibody (anti-CR2 to hTT30 ratio is 2:1). Data represent mean±SEM. As shown in FIG. 14, anti-C5 antibody and hTT30 effectively inhibit CCP and CAP, respectively. The co-treatment of anti-CR2 antibody did not abolish the inhibition of cell lysis by hTT30

### Example 3

### Inhibition of Complement Alternative Pathway by Treatment of Kidney with TT30 Prior to Transplantation Improves Graft Survival

Lewis to Lewis rat orthotropic kidney transplantation was performed with or without treatment of anti-rat C5 monoclonal antibody or hTT30. Rat kidneys were perfused with ice-cold University of Wisconsin solution (UW) with or without therapeutic agent (anti-C5: 200 μg/mL; hTT30: 130 μg/mL, or isotype-matched antibody: 200 μg/mL). Perfusions were performed using a syringe using constant pressure. The kidney was then excised and placed in ice-cold perfusion solution (UW solution with or without therapeutic agents of a same concentration) for the period of cold ischemia at 4° C. for 28 hours. The kidneys were perfused a second time with ice-cold UW solution before transplantation to syngeneic recipients.

### Results:

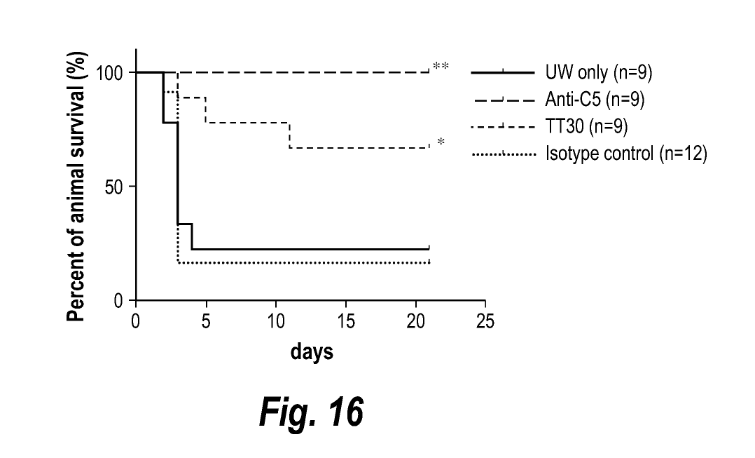

Median survival was 3 days post-transplantation for the rats receiving organs from the control groups, while animals receiving hTT30 or anti-C5 mAb treated organs survived for a median of 21 days. Graft viability was recorded until the time of sacrifice (day 21) and the number of animals transplanted per treatment group is included in parentheses (see FIG. 16, *P<0.05 and **P<0.01 compared with UW group, log-rank test). As in FIG. 16, pretreatment of the organ with hTT30 clearly improved graft survival. Compared to the sudden graft failure at about day 2 to day 3 post transplantation under control treatment, hTT30 pretreatment substantially increased graft survival and sustained this increase until the time of sacrifice. The effect of hTT30 pretreatment is at least above 50-60% of the effect of anit-05 monoclonal antibody pretreatment, which means inhibiting only alternative complement pathway is sufficient to significantly increase graft survival. The different effects between hTT30 and anti-C5 antibody may indicate that inhibiting both classical and alternative complement pathways can further improve graft survival. However, it may also because that the most effective concentrations or dosage regimens of hTT30 were not used in this study. Further experiments will be performed to optimize the hTT30 pretreatment.

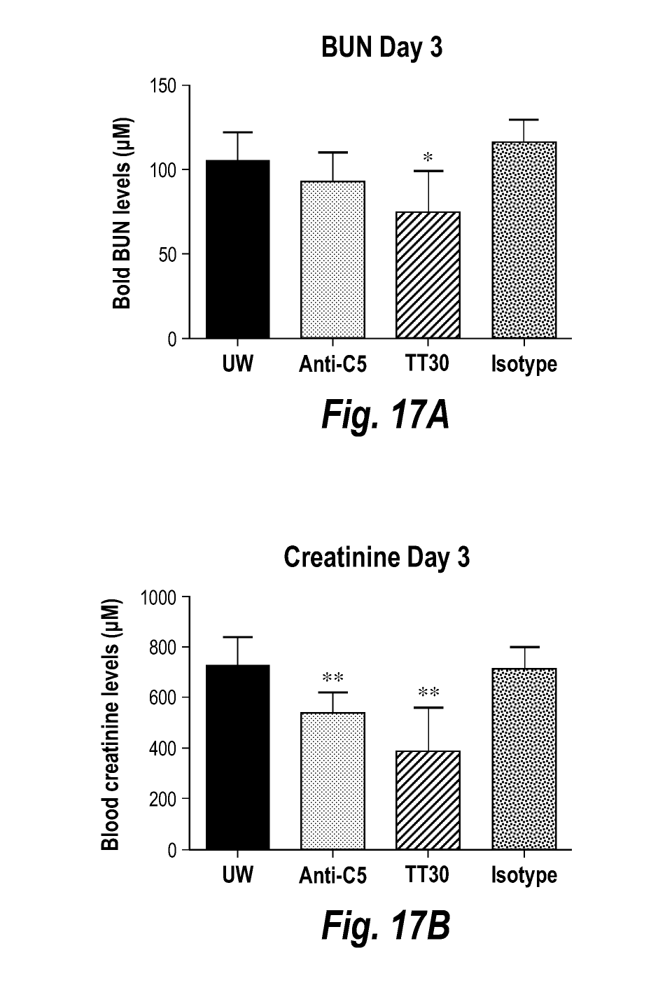

The renal function after transplantation was also tested. The creatinine and BUN levels of surviving animals at day 3 post-transplantation were measured and compared. As shown in FIG. 17, both hTT30 and anti-C5 monoclonal antibody pretreatment decreased blood creatinine and BUN levels significantly. hTT30 pretreatment was even more effective than anti-C5 antibody in this study. Therefore, hTT30 pretreatment is an effective way to improve renal function after transplantation. Data are means±SEM (n=7 to 9 in each group) and significantly different by t-test (*P<0.05 and **P<0.01 compared with UW group).

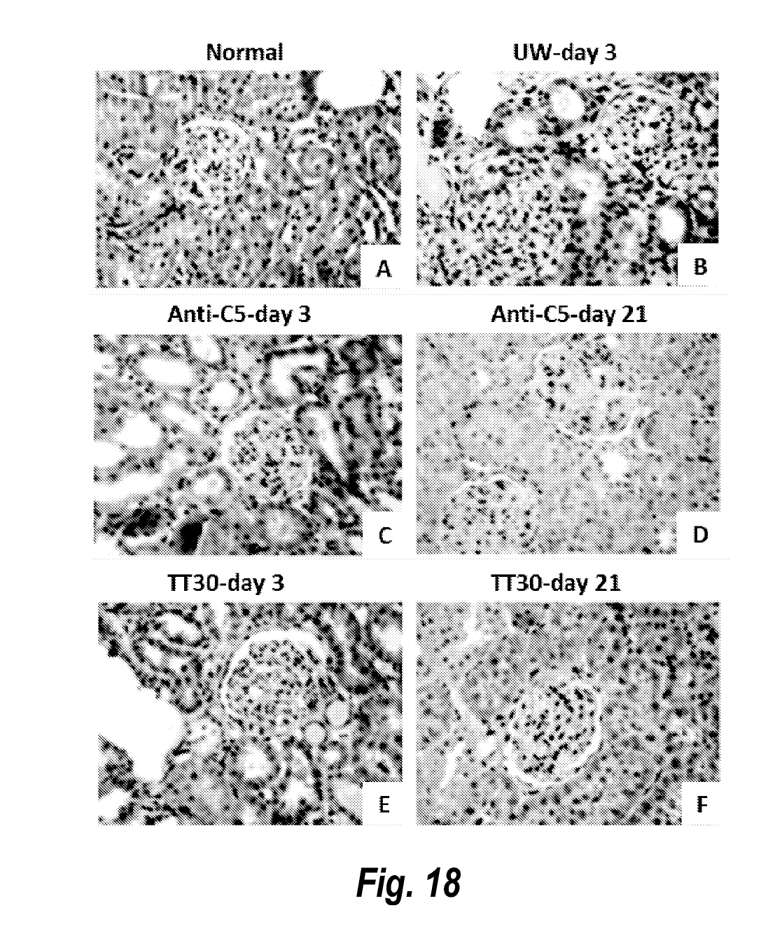

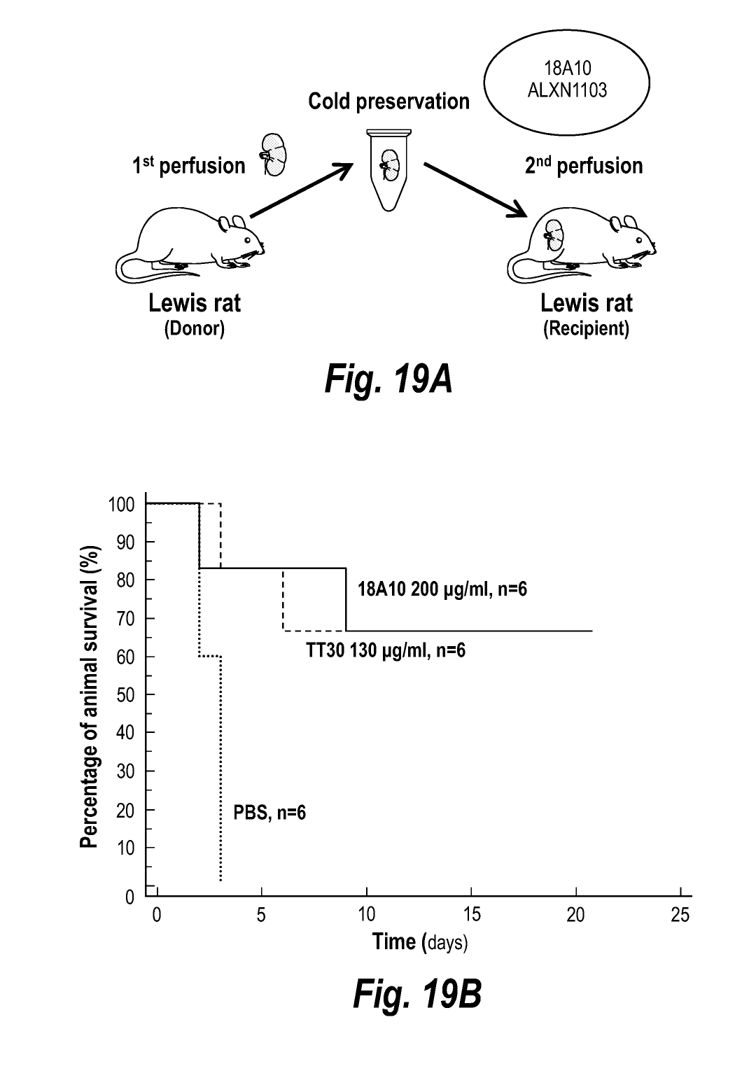

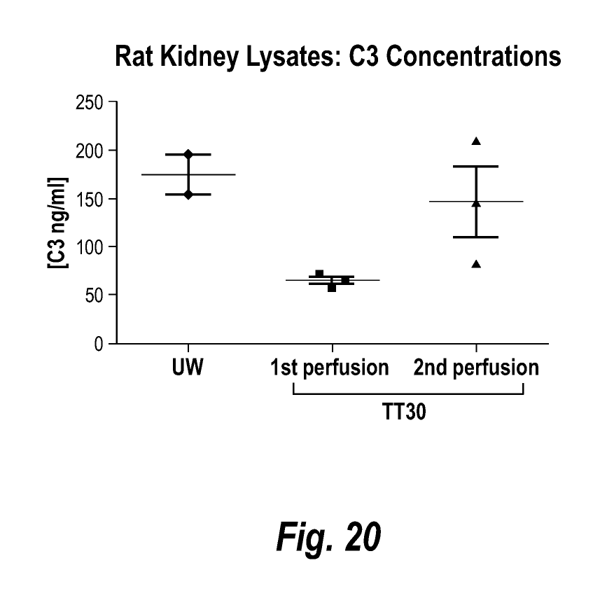

Hematoxylin eosin-stained histological sections (20X) was performed to further illustrate the effect of complement inhibition on ischemia-reperfusion injury in rat renal isografts. As shown in FIG. 18, typical IRI histological features, such as tubular dilation, swelling and necrosis and severe leukocyte infiltration, were observed for UW solution-treated isografts removed on day 3 post-transplantation, compared to normal kidneys. However, both anti-C5 monoclonal antibody and hTT30-treated isografts at day 3 post-transplantation showed reduced cell infiltration, less tubular injury and relatively normal glomeruli morphology. At day 21, the histology of the both complement inhibitors-treated isografts were close to normal, with less damage within tubular epithelial cells and glomerular cells. One the contrary, no animals from the UW treated control group survived to day 21. These histological comparisons clearly show that TT30 pretreatment significantly reduces early tissue ischemia-reperfusion damages and improves renal survival in rat. Notably hTT30 pretreatment in this study had comparable curing effect as anti-C5 antibody treatment.

### Conclusion:

The data suggest a key role for therapeutic inhibition of the complement alternative pathway in the prevention of ischemia-reperfusion injury in the rat kidney transplant model for DGF. Treatment of the donor organ with hTT30 reduced IRI associated acute kidney injury allowing for survival of the graft. On the basis of observations, the use of hTT30 may improve the clinical course of early post-transplant complications, potentially influencing long-term graft function and survival.

### Example 4

### Inhibition of Both Terminal and Alternative Complement Pathways Prior to Transplantation Improves Graft Survival

The following study was performed to measure the increase in graft survival and reduction in IRI following treatment of donor organs with complement inhibitors right before transplantation. Donor kidneys were perfused and preserved in UW solution in the absence of complement inhibitors. After 28 h cold storage at 4° C., donor kidneys were re-perfused with fresh UW solution in the presence of either TT30 (130 μg/mL) or anti-rat C5 mAb 18A10 (200 μg/mL). UW solution alone was used as a control. The donor kidneys were stored in the perfusate for 45 min at 4° C. prior to transplantation without further flushing, so that the complement inhibitors remained in the organ for transplant.

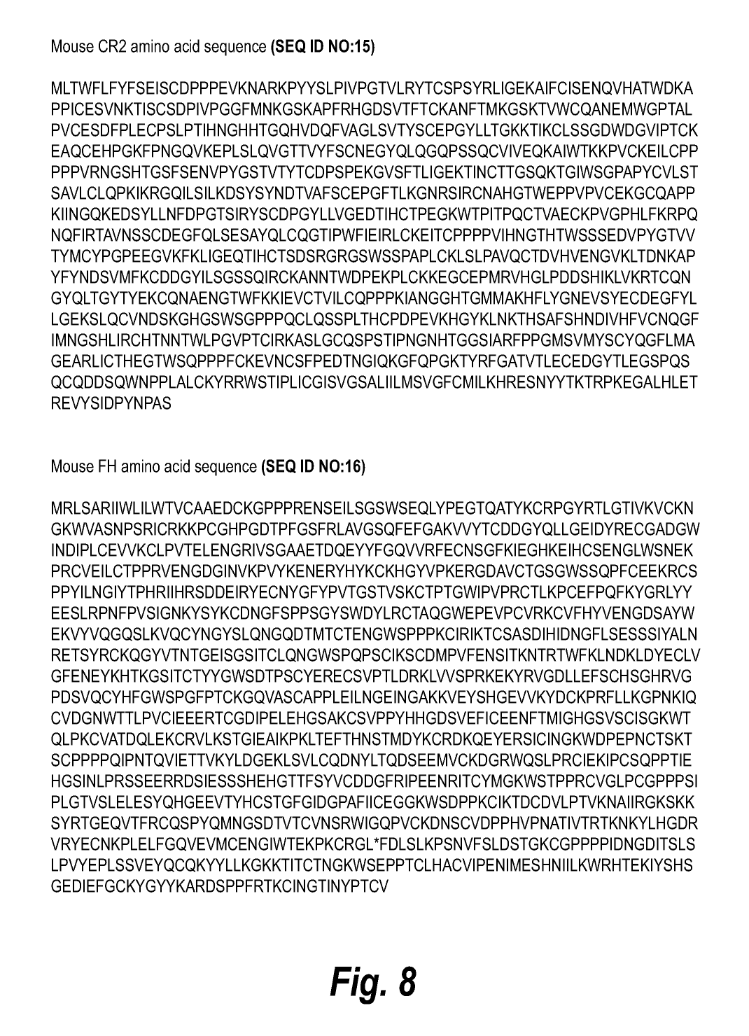

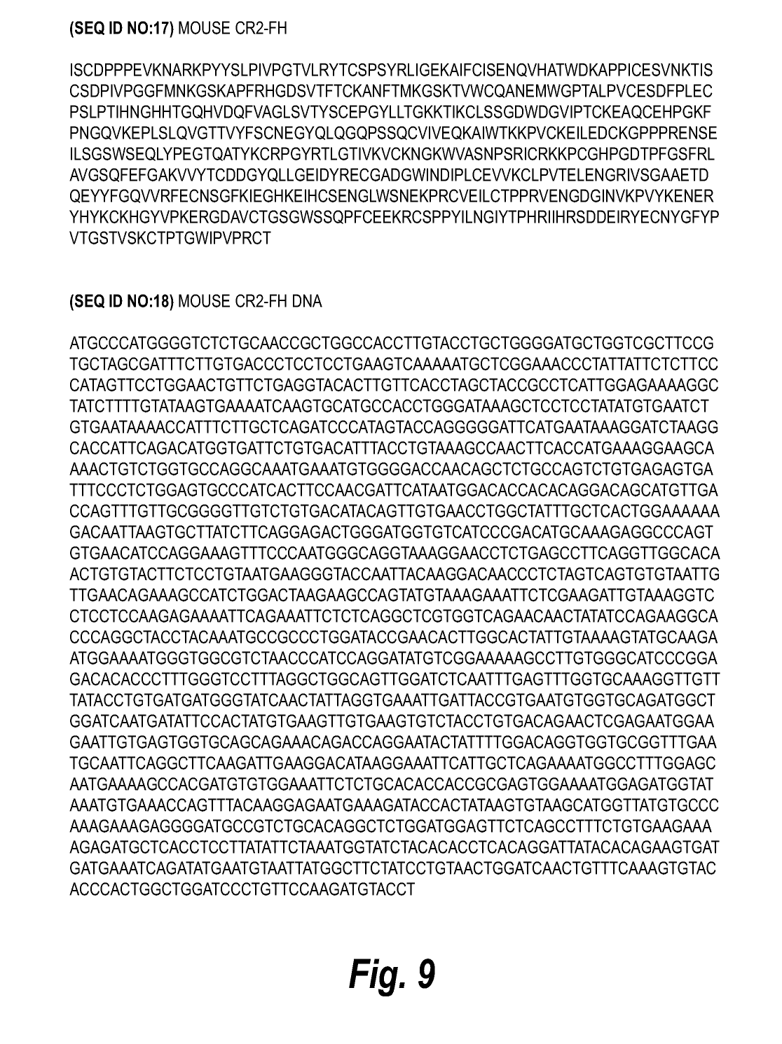

As shown in FIG. 8, animals grafted with TT30 or 18A10-treated kidneys had significantly increased graft survival compared to animals grafted with control-treated kidneys (66.7% for TT30 (4 of 6) and 66.7% for 18A10 (4 of 6) versus 0% (0 of 6) for UW solution alone; P<0.01). These data demonstrate that treatment of donor organ with either alternative pathway inhibitors or terminal pathway inhibitors, particularly low molecular weight inhibitors (e.g., 70 kDa or less) and/or inhibitors which exhibit a short half-life (e.g., less than 10 days), such as TT30 and 18A10 (single chain antibody), prior to transplantation can reduce IRI and increase graft survival.

### Example 5

### Inhibition of Alternative Complement Pathway in Donor Organ Reduces Complement C3 Level in Kidney

The following study was performed to test whether alternative pathway inhibitor treatment of donor organs inhibits complement activation in the organs. TT30 (130 μg/mL in UW solution) was applied to donor organs either in procurement perfusion (first perfusion) and 28 h preservation, or in post-ischemia perfusion (the perfusion after 28 h cold ischemia, i.e., second perfusion) and 45 min preservation. The kidneys were homogenized and the lysates was used for complement C3 measurement by ELISA.

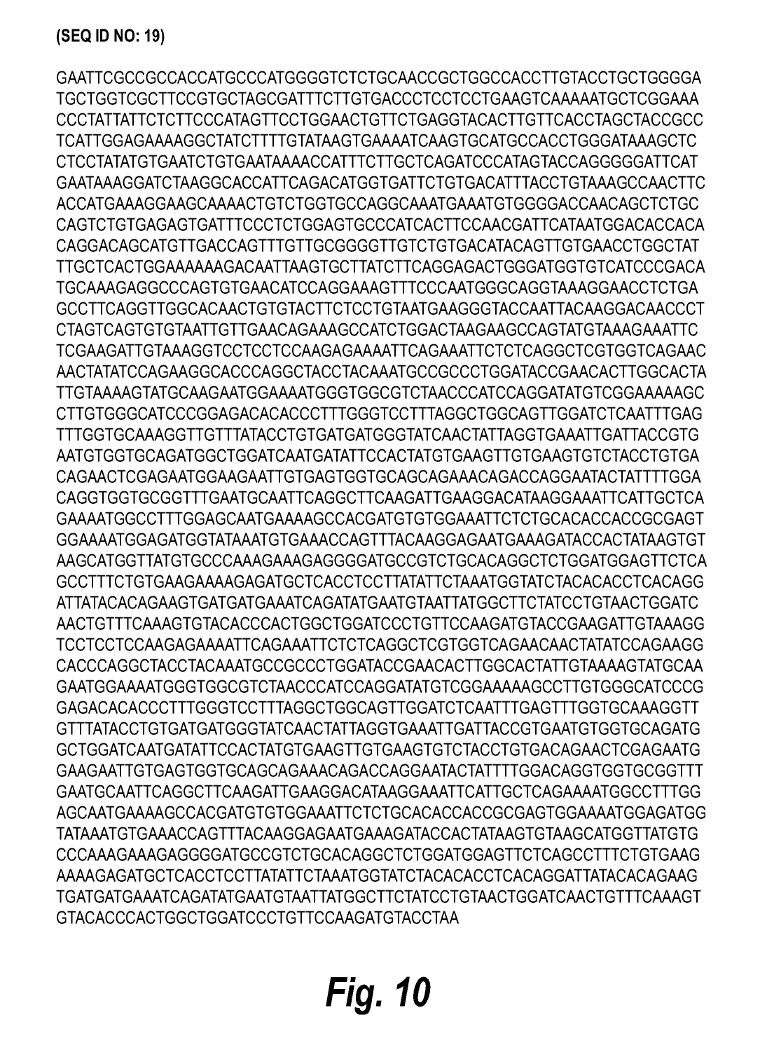

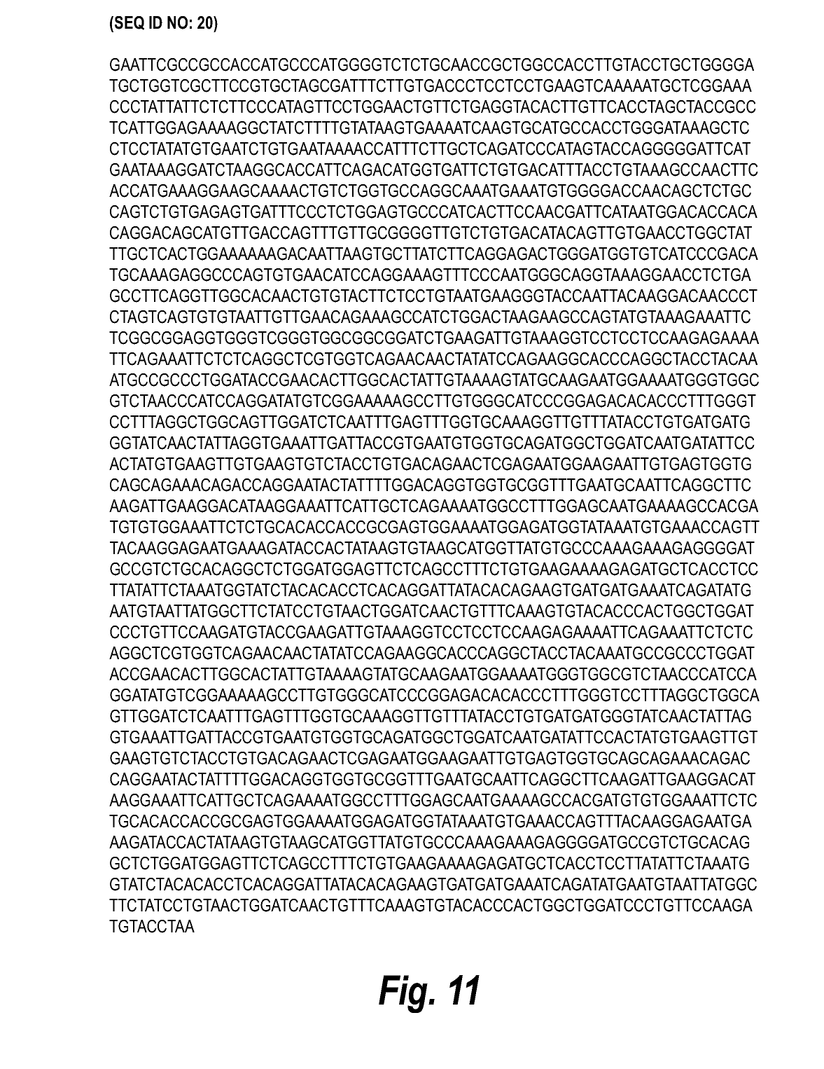

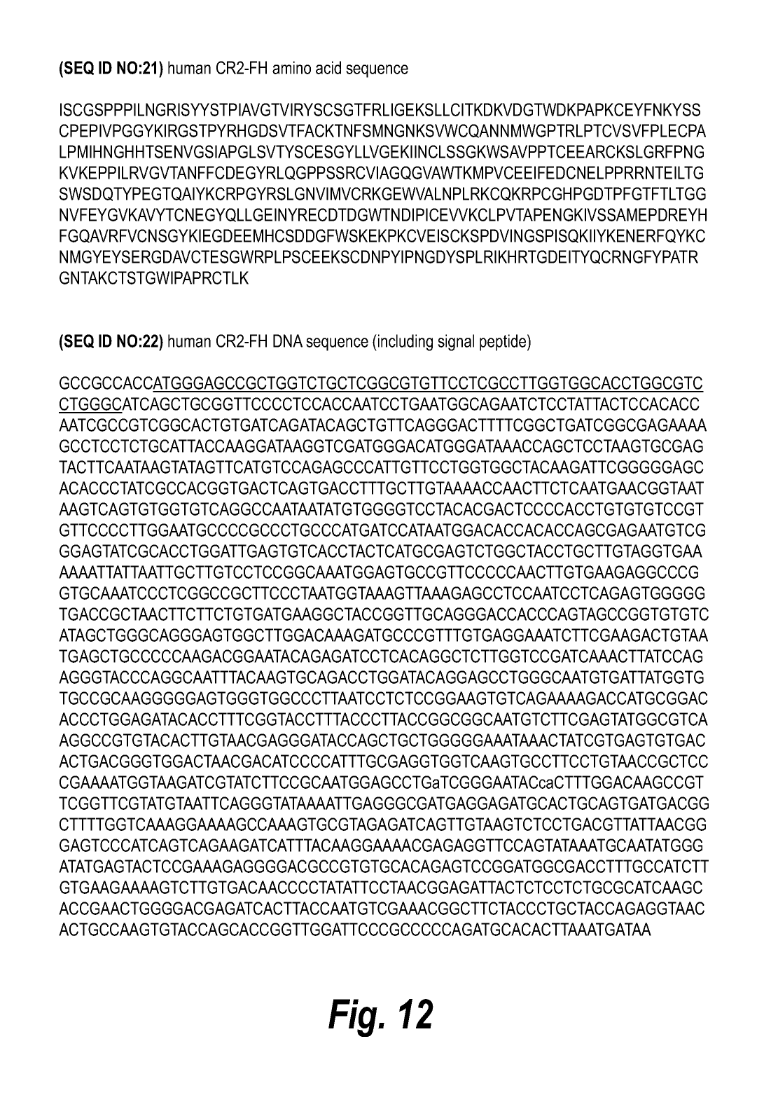

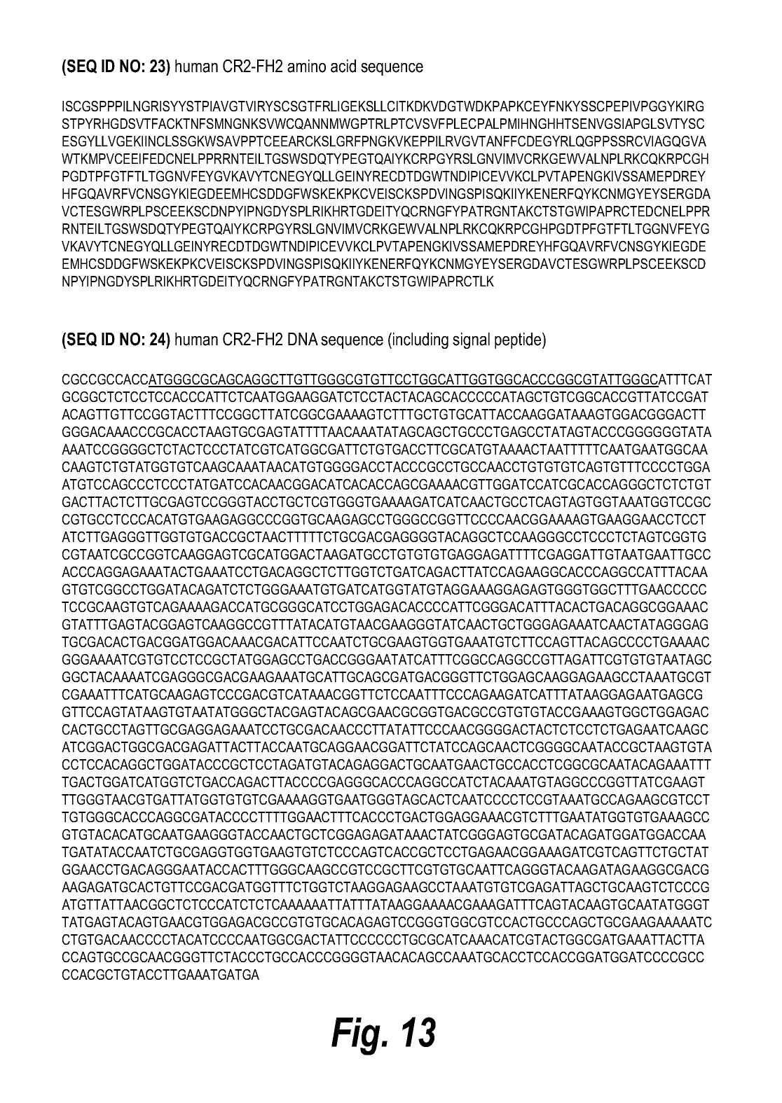

As shown in FIG. 10, TT30 treatment in procurement perfusion and 28 h preservation significantly reduced C3 level compared to UW solution alone. Use of TT30 treatment in post-ischemia perfusion and 45 min preservation did not achieve significant effect in reducing C3 level compared to UW solution control. These results demonstrated that inhibition of the alternative pathway of complement activation in donor organs, particularly using low molecular weight inhibitors (e.g., 70 kDa or less) and/or inhibitors which exhibit a short half-life (e.g., less than 10 days), such as TT30 and 18A10, can effectively prevent complement activation in the organ.

The foregoing examples are merely illustrative and should not be construed as limiting the scope of the present disclosure in any way.

The contents of all references, Genbank entries, patents and published patent applications cited throughout this application are expressly incorporated herein by reference.

### LIST OF REFERENCES

The publications and other materials used herein to illuminate the background of the disclosure, and in particular, cases to provide additional details respecting the practice, are incorporated herein by reference in their entirety, and for convenience, are referenced by author and date in the text and respectively grouped in the following List of References.
Abbas A K, et al. (2000). Cellular and Molecular Immunology (4^{th} edition), p. 363-383 (W.B. Saunders Company, New York).
Arp et al. (1996). J. Virol. 70:7349-7359.
Baldwin et al. (2001). Immunity 14:369-376.
Böhmig G A, et al. (2000). Am. J. Kidney Dis. 35:667-673.
Brauer et al. (1995). Transplantation 59:288-293.
Changelian P S, et al. (2003). Science 302:875-878.
Collard et al. (1997). Circulation 96:326-333.
Collins et al. (1999). J. Am. Soc. Nephrol. 10:2208-2214.
Fearon (1983). In Intensive Review of Internal Medicine, 2^{nd} Ed. Fanta and Minaker, eds. Brigham and Women's and Beth Israel Hospitals.
Forbes et al. (1978). Lab. Invest. 39:463-470.
Frei Y, et al. (1987). Mol. Cell. Probes 1:141-149.
Gloor (2005). Contrib. Nephrol. 146:11-21.
Glotz et al. (1993). Transplantation 56:335-337.
Glotz D, et al. (2002). Am. J. Transplant. 2:758-760.
Hakim et al. (1990). Am. J. Kidney Dis. 16:423-431.
Halloran P F, et al. (1992). Transplantation 53:550-555.
Halloran (2003). Am. J. Transplant. 3:639-640.
Haviland D L, et al. (1991). J. Immunol. 146:362-368.
Hiesse C, et al. (1992). Nephrol. Dial. Transplant. 7:944-951.
Hillmen P, et al. (2004). New Engl. J. Med. 350:552-559.
Jeannet M, et al. (1970). New Engl. J. Med. 282:111-117.
Jones P T, et al. (1986). Nature 321:522-525.
Jose et al. (1983). J. Exp. Med. 158:2177-2182.
Kirschfink (2001). Immunol. Rev. 180:177-189.
Kobayashi et al. (1999). J. Biol. Chem. 274:28660-28668.
Kriaa et al. (1995). Nephrol. Dial. Transplant. 10 Suppl. 6:108-110.
Kroshus et al. (1995). Transplantation 60:1194-1202.
Kupiec-Weglinski (1996). Ann. Transplant. 1:34-40.
Kupin et al. (1991). Transplantation 51:324-329.
Liszewski (1993). Fundamental Immunology pp. 917-939.
Mauiyyedi et al. (2002). Curr. Opin. Nephrol. Hypertens. 11:609-618.
Mehra et al. (2003). Curr. Opin. Cardiol. 18:153-158.
Minta J O and Man D P (1977). J. Immunol. 119:1597-1602.
Montgomery R A, et al. (2000). Transplantation 70:887-895.
Olfert et al. (1993) Guide to the care and use of experimental animals (Vol. 1). Ottawa: Association of Universities and Colleges of Canada 1.
Opelz G (1992). Transplant. Proc. 24:2342-2355.
OPTN/SRTR Annual Report (2002). Chapter 1 of the Annual Report produced by the Scientific Registry of Transplant Recipients (SRTR) in collaboration with the Organ Procurement and Transplantation Network (OPTN). See http://www.unos.org/data/ar2002/ar02_chapter_one.htm.
Palmer et al. (1989). Lancet 1:10-12.
Papadimitriou et al. (1991). J. Immunol. 147:212-217.
Park W D et al. (2003). Am. J. Transplant 3:952-960.
Persson N H et al. (1995). Transplant. Proc. 27:3466.
Platt et al. (1999). Mol. Immunol. 36:965-971.
Pratt et al. (1996). Am J Pathol 149:2055-2066.
Pratt et al. (2000). Am. J. Pathol. 157:825-831.
Pruitt et al. (1991). J. Surg. Res. 50:350-355.
Regele H, et al. (2001). Nephrol. Dial. Transplant. 16:2058-2066.
Rocha et al. (2003). Transplantation 75:1490-1495.
Ross et al. (1993). Transplantation 55:785-789.
Saadi et al. (1995). J. Exp. Med. 182:1807-1814.
Salama et al. (2001). Am. J. Transplant. 1:260-269.
Schweitzer et al. (2000). Transplantation 70:1531-1536.
Sonnenday et al. (2002). Transplant. Proc. 34:1614-1616.
Stepkowski S M (2000). Exp. Rev. Mol. Med. 21 June, http://www-ermm.cbcu.cam.ac.uk00001769h.htm.
Taube D H, et al. (1984). Lancet 1:824-828.
Tyan et al. (1994). Transplantation 57:553-562.
Vakeva et al. (1998). Circulation 97:2259-2267.
Vogt W, et al. (1989). Molec. Immunol. 26:1133-1142.
Wang et al. (1995). Proc. Natl. Acad. Sci. U.S.A 92:8955-8959.
Wang et al. (1996). Proc. Natl. Acad. Sci. U.S.A 93:8563-8568.
Wang et al. (1999). Transplantation 68:1643-1651.
Wang H, et al. (2003). J. Immunol. 171:3823-3836.
Warren et al. (2004). Am. J. Transplant. 4:561-568.
Wetsel R A and Kolb W P (1982). J. Immunol. 128:2209-2216.
Wurzner R, et al. (1991). Complement Inflamm. 8:328-340.
Yamamoto K I and Gewurz G (1978). J. Immunol. 120:2008-2015.

The contents of all references cited herein are incorporated by reference in their entirety.

### Sequence Summary

| | |
|---|---|
| SEQ ID NO: 1 | MGAAGLLGVFLALVAPGVLGISCGSPPPILNGRISYYSTPIAVGTVIRYSCSG |
| Amino acid | TFRLIGEKSLLCITKDKVDGTWDKPAPKCEYFNKYSSCPEPIVPGGYKIRGS |
| sequence of | TPYRHGDSVTFACKTNFSMNGNKSVWCQANNMWGPTRLPTCVSVFPLEC |
| human CR2 | PALPMIHNGHHTSENVGSIAPGLSVTYSCESGYLLVGEKIINCLSSGKWSAV |
| | PPTCEEARCKSLGRFPNGKVKEPPILRVGVTANFFCDEGYRLQGPPSSRCVI |
| | AGQGVAWTKMPVCEEIFCPSPPPILNGRHIGNSLANVSYGSIVTYTCDPDPE |
| | EGVNFILIGESTLRCTVDSQKTGTWSGPAPRCELSTSAVQCPHPQILRGRMV |
| | SGQKDRYTYNDTVIFACMFGFTLKGSKQIRCNAQGTWEPSAPVCEKECQA |
| | PPNILNGQKEDRHMVRFDPGTSIKYSCNPGYVLVGEESIQCTSEGVWTPPV |
| | PQCKVAACEATGRQLLTKPQHQFVRPDVNSSCGEGYKLSGSVYQECQGTI |
| | PWFMEIRLCKEITCPPPPVIYNGAHTGSSLEDFPYGTTVTYTCNPGPERGVE |
| | FSLIGESTIRCTSNDQERGTWSGPAPLCKLSLLAVQCSHVHIANGYKISGKE |
| | APYFYNDTVTFKCYSGFTLKGSSQIRCKRDNTWDPEIPVCEKGCQPPPGLH |
| | HGRHTGGNTVFFVSGMTVDYTCDPGYLLVGNKSIHCMPSGNWSPSAPRC |
| | EETCQHVRQSLQELPAGSRVELVNTSCQDGYQLTGHAYQMCQDAENGIW |
| | FKKIPLCKVIHCHPPPVIVNGKHTGMMAENFLYGNEVSYECDQGFYLLGEK |
| | NCSAEVILKAWILERAFPQCLRSLCPNPEVKHGYKLNKTHSAYSHNDIVYV |
| | DCNPGFIMNGSRVIRCHTDNTWVPGVPTCIKKAFIGCPPPPKTPNGNHTGG |
| | NIARFSPGMSILYSCDQGYLVVGEPLLLCTHEGTWSQPAPHCKEVNCSSPA |
| | DMDGIQKGLEPRKMYQYGAVVTLECEDGYMLEGSPQSQCQSDHQWNPPL |
| | AVCRSRSLAPVLCGIAAGLILLTFLIVITLYVISKHRERNYYTDTSQKEAFHL |
| | EAREVYSVDPYNPAS |
| SEQ ID NO: 2 | MRLLAKIICLMLWAICVAEDCNELPPRRNTEILTGSWSDQTYPEGTQAIYK |
| Amino acid | CRPGYRSLGNVIMVCRKGEWVALNPLRKCQKRPCGHPGDTPFGTFTLTG |
| sequence of | GNVFEYGVKAVYTCNEGYQLLGEINYRECDTDGWTNDIPICEVVKCLPVT |
| human FH | APENGKIVSSAMEPDREYHFGQAVRFVCNSGYKIEGDEEMHCSDDGFWS |
| | KEKPKCVEISCKSPDVINGSPISQKIIYKENERFQYKCNMGYEYSERGDAV |
| | CTESGWRPLPSCEEKSCDNPYIPNGDYSPLRIKHRTGDEITYQCRNGFYPA |
| | TRGNTAKCTSTGWIPAPRCTLKPCDYPDIKHGGLYHENMRRPYFPVAVGK |
| | YYSYYCDEHFETPSGSYWDHIHCTQDGWSPAVPCLRKCYFPYLENGYNQ |
| | NHGRKFVQGKSIDVACHPGYALPKAQTTVTCMENGWSPTPRCIRVKTCSK |
| | SSIDIENGFISESQYTYALKEKAKYQCKLGYVTADGETSGSIRCGKDGWSA |
| | QPTCIKSCDIPVFMNARTKNDFTWFKLNDTLDYECHDGYESNTGSTTGSIV |
| | CGYNGWSDLPICYERECELPKIDVHLVPDRKKDQYKVGEVLKFSCKPGFTI |
| | VGPNSVQCYHFGLSPDLPICKEQVQSCGPPPELLNGNVKEKTKEEYGHSEV |
| | VEYYCNPRFLMKGPNKIQCVDGEWTTLPVCIVEESTCGDIPELEHGWAQLS |
| | SPPYYYGDSVEFNCSESFTMIGHRSITCIHGVWTQLPQCVAIDKLKKCKSSN |
| | LIILEEHLKNKKEFDHNSNIRYRCRGKEGWIHTVCINGRWDPEVNCSMAQIQ |
| | LCPPPPQIPNSHNMTTTLNYRDGEKVSVLCQENYLIQEGEEITCKDGRWQSIP |
| | LCVEKIPCSQPPQIEHGTINSSRSSQESYAHGTKLSYTCEGGFRISEENETTCY |
| | MGKWSSPPQCEGLPCKSPPEISHGVVAHMSDSYQYGEEVTYKCFEGFGIDG |
| | PAIAKCLGEKWSHPPSCIKTDCLSLPSFENAIPMGEKKDVYKAGEQVTYTCA |
| | TYYKMDGASNVTCINSRWTGRPTCRDTSCVNPPTVQNAYIVSRQMSKYPSG |
| | ERVRYQCRSPYEMFGDEEVMCLNGNWTEPPQCKDSTGKCGPPPPIDNGDIT |
| | SFPLSVYAPASSVEYQCQNLYQLEGNKRITCRNGQWSEPPKCLHPCVISREIM |
| | ENYNIALRWTAKQKLYSRTGESVEFVCKRGYRLSSRSHTLRTTCWDGKLEYP |
| | TCAKR |
| SEQ ID NO: 3 | ISCGSPPPILNGRISYYSTPIAVGTVIRYSCSGTFRLIGEKSLLCITKDKVDGTW |
| Amino acid | DKPAPKCEYFNKYSSCPEPIVPGGYKIRGSTPYRHGDSVTFACKTNFSMNGN |
| sequence of | KSVWCQANNINNMWGPTRLPTCVSVFPLECPALPMIHNGHHTSENVGSIAP |
| human CR2- | GLSVTYSCESGYLLVGEKIINCLSSGKWSAVPPTCEEAXCKSLGRFPNGKVK |
| FH | EPPILRVGVTANFFCDEGYRLQGPPSSRCVIAGQGVAWTKMPVCGGGGSGG |
| | GGSCVAEDCNELPPRRNTEILTGSWSDQTYPEGTQAIYKCRPGYRSLGNVIM |
| | VCRKGEWVALNPLRKCQKRPCGHPGDTPFGTFTLTGGNVFEYGVKAVYTC |
| | NEGYQLLGEINYRECDTDGWTNDIPICEVVKCLPVTAPENGKIVSSAMEPDR |
| | EYHFGQAVRFVCNSGYKIEGDEEMHCSDDGFWSKEKPKCVEISCKSPDVIN |
| | GSPISQKIIYKENERFQYKCNMGYEYSERGDAVCTESGWRPLPSCEEKSCDN |
| | PYIPNGDYSPLRIKHRTGDEITYQCRNGFYPATRGNTAKCTSTGWIPAPRCT |
| SEQ ID NO: 4 | ATTTCTTGTGGCTCTCCTCCGCCTATCCTAAATGGCCGGATTAGTTATTAT |
| Nucleic acid | TCTACCCCCATTGCTGTTGGTACCGTGATAAGGTACAGTTGTTCAGGTAC |
| sequence of | CTTCCGCCTCATTGGAGAAAAAAGTCTATTATGCATAACTAAAGACAAA |
| human CR2- | GTGGATGGAACCTGGGATAAACCTGCTCCTAAATGTGAATATTTCAATA |
| FH | AATATTCTTCTTGCCCTGAGCCCATAGTACCAGGAGGATACAAAATTAG |
| | AGGCTCTACACCCTACAGACATGGTGATTCTGTGACATTTGCCTGTAAA |
| | ACCAACTTCTCCATGAACGGAAACAAGTCTGTTTGGTGTCAAGCAAATA |
| | ATATAAATAATATGTGGGGGCCGACACGACTACCAACCTGTGTAAGTGT |
| | TTTCCCTCTCGAGTGTCCAGCACTTCCTATGATCCACAATGGACATCACA |
| | CAAGTGAGAATGTTGGCTCCATTGCTCCAGGATTGTCTGTGACTTACAGC |
| | TGTGAATCTGGTTACTTGCTTGTTGGAGAAAAGATCATTAACTGTTTGTC |
| | TTCGGGAAAATGGAGTGCTGTCCCCCCCACATGTGAAGAGGCACSCTGT |
| | AAATCTCTAGGACGATTTCCCAATGGGAAGGTAAAGGAGCCTCCAATTC |
| | TCCGGGTTGGTGTAACTGCAAACTTTTTCTGTGATGAAGGGTATCGACTG |
| | CAAGGCCCACCTTCTAGTCGGTGTGTAATTGCTGGACAGGGAGTTGCTTG |
| | GACCAAAATGCCAGTATGTGGCGGAGGTGGGTCGGGTGGCGGCGGATCT |
| | TGTGTAGCAGAAGATTGCAATGAACTTCCTCCAAGAAGAAATACAGAA |
| | ATTCTGACAGGTTCCTGGTCTGACCAAACATATCCAGAAGGCACCCAG |
| | GCTATCTATAAATGCCGCCCTGGATATAGATCTCTTGGAAATGTAATAA |
| | TGGTATGCAGGAAGGGAGAATGGGTTGCTCTTAATCCATTAAGGAAAT |
| | GTCAGAAAAGGCCCTGTGGACATCCTGGAGATACTCCTTTTGGTACTTT |
| | TACCCTTACAGGAGGAAATGTGTTTGAATATGGTGTAAAAGCTGTGTAT |
| | ACATGTAATGAGGGGTATCAATTGCTAGGTGAGATTAATTACCGTGAAT |
| | GTGACACAGATGGATGGACCAATGATATTCCTATATGTGAAGTTGTGAA |
| | GTGTTTACCAGTGACAGCACCAGAGAATGGAAAAATTGTCAGTAGTGCA |
| | ATGGAACCAGATCGGGAATACCATTTTGGACAAGCAGTACGGTTTGTAT |
| | GTAACTCAGGCTACAAGATTGAAGGAGATGAAGAAATGCATTGTTCAGA |
| | CGATGGTTTTTGGAGTAAAGAGAAACCAAAGTGTGTGGAAATTTCATGC |
| | AAATCCCCAGATGTTATAAATGGATCTCCTATATCTCAGAAGATTATTTA |
| | TAAGGAGAATGAACGATTTCAATATAAATGTAACATGGGTTATGAATAC |
| | AGTGAAAGAGGAGATGCTGTATGCACTGAATCTGGATGGCGTCCGTTGC |
| | CTTCATGTGAAGAAAAATCATGTGATAATCCTTATATTCCAAATGGTGAC |
| | TACTCACCTTTAAGGATTAAACACAGAACTGGAGATGAAATCACGTACCA |
| | GTGTAGAAATGGTTTTTATCCTGCAACCCGGGGAAATACAGCCAAATGCA |
| | CAAGTACTGGCTGGATACCTGCTCCGAGATGTACCT |
| SEQ ID NO: 5 | ISCGSPPPILNGRISYYSTPIAVGTVIRYSCSGTFRLIGEKSLLCITKDKVDGTW |
| nnn = optional | DKPAPKCEYFNKYSSCPEPIVPGGYKIRGSTPYRHGDSVTFACKTNFSMNGN |
| linker | KSVWCQANNMWGPTRLPTCVSVFPLECPALPMIHNGHHTSENVGSIAPGLS |
| | VTYSCESGYLLVGEKIINCLSSGKWSAVPPTCEEARCKSLGRFPNGKVKEPPI |
| | LRVGVTANFFCDEGYRLQGPPSSRCVIAGQGVAWTKMPVCnnnCVAEDCNE |
| | LPPRRNTEILTGSWSDQTYPEGTQAIYKCRPGYRSLGNVIMVCRKGEWVALN |
| | PLRKCQKRPCGHPGDTPFGTFTLTGGNVFEYGVKAVYTCNEGYQLLGEINYR |
| | ECDTDGWTNDIPICEVVKCLPVTAPENGKIVSSAMEPDREYHFGQAVRFVCN |
| | SGYKIEGDEEMHCSDDGFWSKEKPKCVEISCKSPDVINGSPISQKIIYKENERF |
| | QYKCNMGYEYSERGDAVCTESGWRPLPSCEEKSCDNPYIPNGDYSPLRIKHR |
| | TGDEITYQCRNGFYPATRGNTAKCTSTGWIPAPRCT |
| SEQ ID NO: 6 | ISCGSPPPILNGRISYYSTPIAVGTVIRYSCSGTFRLIGEKSLLCITKDKVDGTWD |
| nnn = optional | KPAPKCEYFNKYSSCPEPIVPGGYKIRGSTPYRHGDSVTFACKTNFSMNGNKS |
| linker | VWCQANNMWGPTRLPTCVSVFPLECPALPMIHNGHHTSENVGSIAPGLSVTY |
| | SCESGYLLVGEKIINCLSSGKWSAVPPTCEEARCKSLGRFPNGKVKEPPILRVG |
| | VTANFFCDEGYRLQGPPSSRCVIAGQGVAWTKMPVCnnnCVAEDCNELPPRR |
| | NTEILTGSWSDQTYPEGTQAIYKCRPGYRSLGNIIMVCRKGEWVALNPLRKC |
| | QKRPCGHPGDTPFGTFTLTGGNVFEYGVKAVYTCNEGYQLLGEINYRECDTD |
| | GWTNDIPICEVVKCLPVTAPENGKIVSSAMEPDREYHFGQAVRFVCNSGYKIE |
| | GDEEMHCSDDGFWSKEKPKCVEISCKSPDVINGSPISQKIIYKENERFQYKCNM |
| | GYEYSERGDAVCTESGWRPLPSCEEKSCDNPYIPNGDYSPLRIKHRTGDEITYQ |
| | CRNGFYPATRGNTAKCTSTGWIPAPRCT |
| SEQ ID NO: 7 | ISCGSPPPILNGRISYYSTPIAVGTVIRYSCSGTFRLIGEKSLLCITKDKVDGTWD |
| nnn = optional | KPAPKCEYFNKYSSCPEPIVPGGYKIRGSTPYRHGDSVTFACKTNFSMNGNKS |
| linker | VWCQANNINNMWGPTRLPTCVSVFPLECPALPMIHNGHHTSENVGSIAPGLS |
| | VTYSCESGYLLVGEKIINCLSSGKWSAVPPTCEEAXCKSLGRFPNGKVKEPPIL |
| | RVGVTANFFCDEGYRLQGPPSSRCVIAGQGVAWTKMPVCnnnEDCNELPPRR |
| | NTEILTGSWSDQTYPEGTQAIYKCRPGYRSLGNVIMVCRKGEWVALNPLRKC |
| | QKRPCGHPGDTPFGTFTLTGGNVFEYGVKAVYTCNEGYQLLGEINYRECDT |
| | DGWTNDIPICEVVKCLPVTAPENGKIVSSAMEPDREYHFGQAVRFVCNSGYK |
| | IEGDEEMHCSDDGFWSKEKPKCVEISCKSPDVINGSPISQKIIYKENERFQYKC |
| | NMGYEYSERGDAVCTESGWRPLPSCEEKSCDNPYIPNGDYSPLRIKHRTGDEI |
| | TYQCRNGFYPATRGNTAKCTSTGWIPAPRCT |
| SEQ ID NO: 8 | ISCGSPPPILNGRISYYSTPIAVGTVIRYSCSGTFRLIGEKSLLCITKDKVDGTWD |
| nnn = optional | KPAPKCEYFNKYSSCPEPIVPGGYKIRGSTPYRHGDSVTFACKTNFSMNGNKS |
| linker | VWCQANNINNMWGPTRLPTCVSVFPLECPALPMIHNGHHTSENVGSIAPGLS |
| | VTYSCESGYLLVGEKIINCLSSGKWSAVPPTCEEAXCKSLGRFPNGKVKEPPIL |
| | RVGVTANFFCDEGYRLQGPPSSRCVIAGQGVAWTKMPVCnnnEDCNELPPRR |
| | NTEILTGSWSDQTYPEGTQAIYKCRPGYRSLGNIIMVCRKGEWVALNPLRKC |
| | QKRPCGHPGDTPFGTFTLTGGNVFEYGVKAVYTCNEGYQLLGEINYRECDTD |
| | GWTNDIPICEVVKCLPVTAPENGKIVSSAMEPDREYHFGQAVRFVCNSGYKIE |
| | GDEEMHCSDDGFWSKEKPKCVEISCKSPDVINGSPISQKIIYKENERFQYKCN |
| | MGYEYSERGDAVCTESGWRPLPSCEEKSCDNPYIPNGDYSPLRIKHRTGDEIT |
| | YQCRNGFYPATRGNTAKCTSTGWIPAPRCT |
| SEQ ID NO: 9 | ISCGSPPPILNGRISYYSTPIAVGTVIRYSCSGTFRLIGEKSLLCITKDKVDGTWD |
| nnn = optional | KPAPKCEYFNKYSSCPEPIVPGGYKIRGSTPYRHGDSVTFACKTNFSMNGNKS |
| linker | VWCQANNMWGPTRLPTCVSVFPLECPALPMIHNGHHTSENVGSIAPGLSVTY |
| | SCESGYLLVGEKIINCLSSGKWSAVPPTCEEARCKSLGRFPNGKVKEPPILRVG |
| | VTANFFCDEGYRLQGPPSSRCVIAGQGVAWTKMPVCnnnEDCNELPPRRNTEIL |
| | TGSWSDQTYPEGTQAIYKCRPGYRSLGNVIMVCRKGEWVALNPLRKCQKRPC |
| | GHPGDTPFGTFTLTGGNVFEYGVKAVYTCNEGYQLLGEINYRECDTDGWTND |
| | IPICEVVKCLPVTAPENGKIVSSAMEPDREYHFGQAVRFVCNSGYKIEGDEEMH |
| | CSDDGFWSKEKPKCVEISCKSPDVINGSPISQKIIYKENERFQYKCNMGYEYSER |
| | GDAVCTESGWRPLPSCEEKSCDNPYIPNGDYSPLRIKHRTGDEITYQCRNGFYP |
| | ATRGNTAKCTSTGWIPAPRCT |
| SEQ ID | ISCGSPPPILNGRISYYSTPIAVGTVIRYSCSGTFRLIGEKSLLCITKDKVDGTWDK |
| NO: 10 | PAPKCEYFNKYSSCPEPIVPGGYKIRGSTPYRHGDSVTFACKTNFSMNGNKSVW |
| nnn = optional | CQANNMWGPTRLPTCVSVFPLECPALPMIHNGHHTSENVGSIAPGLSVTYSCES |
| linker | GYLLVGEKIINCLSSGKWSAVPPTCEEARCKSLGRFPNGKVKEPPILRVGVTANF |
| | FCDEGYRLQGPPSSRCVIAGQGVAWTKMPVCnnnEDCNELPPRRNTEILTGSWSD |
| | QTYPEGTQAIYKCRPGYRSLGNIIMVCRKGEWVALNPLRKCQKRPCGHPGDTPF |
| | GTFTLTGGNVFEYGVKAVYTCNEGYQLLGEINYRECDTDGWTNDIPICEVVKCL |
| | PVTAPENGKIVSSAMEPDREYHFGQAVRFVCNSGYKIEGDEEMHCSDDGFWSKE |
| | KPKCVEISCKSPDVINGSPISQKIIYKENERFQYKCNMGYEYSERGDAVCTESGWR |
| | PLPSCEEKSCDNPYIPNGDYSPLRIKHRTGDEITYQCRNGFYPATRGNTAKCTSTG |
| | WIPAPRCT |
| SEQ ID | MPMGSLQPLATLYLLGMLVAS |
| NO: 11 | |
| CD5 peptide | |
| sequence | |
| SEQ ID | ATGCCCATGGGGTCTCTGCAACCGCTGGCCACCTTGTACCTGCTGGGGATGC |
| NO: 12 | TGGTCGCTTCCTGCCTCGGA |
| CD5 | |
| nucleotide | |
| sequence | |
| SEQ ID | MGAAGLLGVFLALVAPG |
| NO: 13 | |
| CR2 peptide | |
| sequence | |
| SEQ ID | ATGGGCGCCGCGGGCCTGCTCGGGGTTTTCTTGGCTCTCGTCGCACCGGG |
| NO: 14 | GGTCCTCGGG |
| CR2 | |
| nucleotide | |
| sequence | |
| SEQ ID | MLTWFLFYFSEISCDPPPEVKNARKPYYSLPIVPGTVLRYTCSPSYRLIGEKAIF |
| NO: 15 | CISENQVHATWDKAPPICESVNKTISCSDPIVPGGFMNKGSKAPFRHGDSVTFT |
| Mouse CR2 | CKANFTMKGSKTVWCQANEMWGPTALPVCESDFPLECPSLPTIHNGHHTGQH |
| amino acid | VDQFVAGLSVTYSCEPGYLLTGKKTIKCLSSGDWDGVIPTCKEAQCEHPGKFP |
| sequence | NGQVKEPLSLQVGTTVYFSCNEGYQLQGQPSSQCVIVEQKAIWTKKPVCKEIL |
| | CPPPPPVRNGSHTGSFSENVPYGSTVTYTCDPSPEKGVSFTLIGEKTINCTTGSQ |
| | KTGIWSGPAPYCVLSTSAVLCLQPKIKRGQILSILKDSYSYNDTVAFSCEPGFTL |
| | KGNRSIRCNAHGTWEPPVPVCEKGCQAPPKIINGQKEDSYLLNFDPGTSIRYSC |
| | DPGYLLVGEDTIHCTPEGKWTPITPQCTVAECKPVGPHLFKRPQNQFIRTAVNS |
| | SCDEGFQLSESAYQLCQGTIPWFIEIRLCKEITCPPPPVIHNGTHTWSSSEDVPYG |
| | TVVTYMCYPGPEEGVKFKLIGEQTIHCTSDSRGRGSWSSPAPLCKLSLPAVQCT |
| | DVHVENGVKLTDNKAPYFYNDSVMFKCDDGYILSGSSQIRCKANNTWDPEKP |
| | LCKKEGCEPMRVHGLPDDSHIKLVKRTCQNGYQLTGYTYEKCQNAENGTWFK |
| | KIEVCTVILCQPPPKIANGGHTGMMAKHFLYGNEVSYECDEGFYLLGEKSLQCV |
| | NDSKGHGSWSGPPPQCLQSSPLTHCPDPEVKHGYKLNKTHSAFSHNDIVHFVCN |
| | QGFIMNGSHLIRCHTNNTWLPGVPTCIRKASLGCQSPSTIPNGNHTGGSIARFPPG |
| | MSVMYSCYQGFLMAGEARLICTHEGTWSQPPPFCKEVNCSFPEDTNGIQKGFQP |
| | GKTYRFGATVTLECEDGYTLEGSPQSQCQDDSQWNPPLALCKYRRWSTIPLICG |
| | ISVGSALIILMSVGFCMILKHRESNYYTKTRPKEGALHLETREVYSIDPYNPAS |
| SEQ ID | MRLSARIIWLILWTVCAAEDCKGPPPRENSEILSGSWSEQLYPEGTQATYKCRPG |
| NO: 16 | YRTLGTIVKVCKNGKWVASNPSRICRKKPCGHPGDTPFGSFRLAVGSQFEFGAK |
| Mouse FH | VVYTCDDGYQLLGEIDYRECGADGWINDIPLCEVVKCLPVTELENGRIVSGAAE |
| amino acid | TDQEYYFGQVVRFECNSGFKIEGHKEIHCSENGLWSNEKPRCVEILCTPPRVENG |
| sequence | DGINVKPVYKENERYHYKCKHGYVPKERGDAVCTGSGWSSQPFCEEKRCSPPY |
| | ILNGIYTPHRIIHRSDDEIRYECNYGFYPVTGSTVSKCTPTGWIPVPRCTLKPCEFP |
| | QFKYGRLYYEESLRPNFPVSIGNKYSYKCDNGFSPPSGYSWDYLRCTAQGWEPE |
| | VPCVRKCVFHYVENGDSAYWEKVYVQGQSLKVQCYNGYSLQNGQDTMTCTE |
| | NGWSPPPKCIRIKTCSASDIHIDNGFLSESSSIYALNRETSYRCKQGYVTNTGEISG |
| | SITCLQNGWSPQPSCIKSCDMPVFENSITKNTRTWFKLNDKLDYECLVGFENEYK |
| | HTKGSITCTYYGWSDTPSCYERECSVPTLDRKLVVSPRKEKYRVGDLLEFSCHSG |
| | HRVGPDSVQCYHFGWSPGFPTCKGQVASCAPPLEILNGEINGAKKVEYSHGEVV |
| | KYDCKPRFLLKGPNKIQCVDGNWTTLPVCIEEERTCGDIPELEHGSAKCSVPPYH |
| | HGDSVEFICEENFTMIGHGSVSCISGKWTQLPKCVATDQLEKCRVLKSTGIEAIKP |
| | KLTEFTHNSTMDYKCRDKQEYERSICINGKWDPEPNCTSKTSCPPPPQIPNTQVIE |
| | TTVKYLDGEKLSVLCQDNYLTQDSEEMVCKDGRWQSLPRCIEKIPCSQPPTIEHG |
| | SINLPRSSEERRDSIESSSHEHGTTFSYVCDDGFRIPEENRITCYMGKWSTPPRCVG |
| | LPCGPPPSIPLGTVSLELESYQHGEEVTYHCSTGFGIDGPAFIICEGGKWSDPPKCIK |
| | TDCDVLPTVKNAIIRGKSKKSYRTGEQVTFRCQSPYQMNGSDTVTCVNSRWIGQP |
| | VCKDNSCVDPPHVPNATIVTRTKNKYLHGDRVRYECNKPLELFGQVEVMCENGI |
| | WTEKPKCRGL*FDLSLKPSNVFSLDSTGKCGPPPPIDNGDITSLSLPVYEPLSSVEY |
| | QCQKYYLLKGKKTITCTNGKWSEPPTCLHACVIPENIMESHNIILKWRHTEKIYSH |
| | SGEDIEFGCKYGYYKARDSPPFRTKCINGTINYPTCV |
| SEQ ID | ISCDPPPEVKNARKPYYSLPIVPGTVLRYTCSPSYRLIGEKAIFCISENQVHATW |
| NO: 17 | DKAPPICESVNKTISCSDPIVPGGFMNKGSKAPFRHGDSVTFTCKANFTMKGSK |
| Mouse CR2- | TVWCQANEMWGPTALPVCESDFPLECPSLPTIHNGHHTGQHVDQFVAGLSVT |
| FH | YSCEPGYLLTGKKTIKCLSSGDWDGVIPTCKEAQCEHPGKFPNGQVKEPLSLQ |
| | VGTTVYFSCNEGYQLQGQPSSQCVIVEQKAIWTKKPVCKEILEDCKGPPPREN |
| | SEILSGSWSEQLYPEGTQATYKCRPGYRTLGTIVKVCKNGKWVASNPSRICRK |
| | KPCGHPGDTPFGSFRLAVGSQFEFGAKVVYTCDDGYQLLGEIDYRECGADGW |
| | INDIPLCEVVKCLPVTELENGRIVSGAAETDQEYYFGQVVRFECNSGFKIEGHK |
| | EIHCSENGLWSNEKPRCVEILCTPPRVENGDGINVKPVYKENERYHYKCKHGY |
| | VPKERGDAVCTGSGWSSQPFCEEKRCSPPYILNGIYTPHRIIHRSDDEIRYECNY |
| | GFYPVTGSTVSKCTPTGWIPVPRCT |
| SEQ ID | ATGCCCATGGGGTCTCTGCAACCGCTGGCCACCTTGTACCTGCTGGGGATG |
| NO: 18 | CTGGTCGCTTCCGTGCTAGCGATTTCTTGTGACCCTCCTCCTGAAGTCAAAA |
| Mouse CR2- | ATGCTCGGAAACCCTATTATTCTCTTCCCATAGTTCCTGGAACTGTTCTGAG |
| FH DNA | GTACACTTGTTCACCTAGCTACCGCCTCATTGGAGAAAAGGCTATCTTTTGT |
| | ATAAGTGAAAATCAAGTGCATGCCACCTGGGATAAAGCTCCTCCTATATGT |
| | GAATCTGTGAATAAAACCATTTCTTGCTCAGATCCCATAGTACCAGGGGGA |
| | TTCATGAATAAAGGATCTAAGGCACCATTCAGACATGGTGATTCTGTGACA |
| | TTTACCTGTAAAGCCAACTTCACCATGAAAGGAAGCAAAACTGTCTGGTGC |
| | CAGGCAAATGAAATGTGGGGACCAACAGCTCTGCCAGTCTGTGAGAGTGA |
| | TTTCCCTCTGGAGTGCCCATCACTTCCAACGATTCATAATGGACACCACAC |
| | AGGACAGCATGTTGACCAGTTTGTTGCGGGGTTGTCTGTGACATACAGTTG |
| | TGAACCTGGCTATTTGCTCACTGGAAAAAAGACAATTAAGTGCTTATCTTC |
| | AGGAGACTGGGATGGTGTCATCCCGACATGCAAAGAGGCCCAGTGTGAAC |
| | ATCCAGGAAAGTTTCCCAATGGGCAGGTAAAGGAACCTCTGAGCCTTCAG |
| | GTTGGCACAACTGTGTACTTCTCCTGTAATGAAGGGTACCAATTACAAGGA |
| | CAACCCTCTAGTCAGTGTGTAATTGTTGAACAGAAAGCCATCTGGACTAAG |
| | AAGCCAGTATGTAAAGAAATTCTCGAAGATTGTAAAGGTCCTCCTCCAAGA |
| | GAAAATTCAGAAATTCTCTCAGGCTCGTGGTCAGAACAACTATATCCAGAA |
| | GGCACCCAGGCTACCTACAAATGCCGCCCTGGATACCGAACACTTGGCACT |
| | ATTGTAAAAGTATGCAAGAATGGAAAATGGGTGGCGTCTAACCCATCCAGG |
| | ATATGTCGGAAAAAGCCTTGTGGGCATCCCGGAGACACACCCTTTGGGTCC |
| | TTTAGGCTGGCAGTTGGATCTCAATTTGAGTTTGGTGCAAAGGTTGTTTATA |
| | CCTGTGATGATGGGTATCAACTATTAGGTGAAATTGATTACCGTGAATGTG |
| | GTGCAGATGGCTGGATCAATGATATTCCACTATGTGAAGTTGTGAAGTGTC |
| | TACCTGTGACAGAACTCGAGAATGGAAGAATTGTGAGTGGTGCAGCAGAA |
| | ACAGACCAGGAATACTATTTTGGACAGGTGGTGCGGTTTGAATGCAATTCA |
| | GGCTTCAAGATTGAAGGACATAAGGAAATTCATTGCTCAGAAAATGGCCTT |
| | TGGAGCAATGAAAAGCCACGATGTGTGGAAATTCTCTGCACACCACCGCGA |
| | GTGGAAAATGGAGATGGTATAAATGTGAAACCAGTTTACAAGGAGAATGA |
| | AAGATACCACTATAAGTGTAAGCATGGTTATGTGCCCAAAGAAAGAGGGG |
| | ATGCCGTCTGCACAGGCTCTGGATGGAGTTCTCAGCCTTTCTGTGAAGAAA |
| | AGAGATGCTCACCTCCTTATATTCTAAATGGTATCTACACACCTCACAGGAT |
| | TATACACAGAAGTGATGATGAAATCAGATATGAATGTAATTATGGCTTCTAT |
| | CCTGTAACTGGATCAACTGTTTCAAAGTGTACACCCACTGGCTGGATCCCTG |
| | TTCCAAGATGTACCT |
| SEQ ID | GAATTCGCCGCCACCATGCCCATGGGGTCTCTGCAACCGCTGGCCACCTTGTACCT |
| NO: 19 | GCTGGGGATGCTGGTCGCTTCCGTGCTAGCGATTTCTTGTGACCCTCCTCCTGAA |
| Exemplary | GTCAAAAATGCTCGGAAACCCTATTATTCTCTTCCCATAGTTCCTGGAACTGTTC |
| DNA | TGAGGTACACTTGTTCACCTAGCTACCGCCTCATTGGAGAAAAGGCTATCTTTTG |
| sequence of | TATAAGTGAAAATCAAGTGCATGCCACCTGGGATAAAGCTCCTCCTATATGTGA |
| CR2NLFHFH, | ATCTGTGAATAAAACCATTTCTTGCTCAGATCCCATAGTACCAGGGGGATTCATG |
| a mouse CR2- | AATAAAGGATCTAAGGCACCATTCAGACATGGTGATTCTGTGACATTTACCTGTA |
| FH fusion | AAGCCAACTTCACCATGAAAGGAAGCAAAACTGTCTGGTGCCAGGCAAATGAAA |
| protein | TGTGGGGACCAACAGCTCTGCCAGTCTGTGAGAGTGATTTCCCTCTGGAGTGCCC |
| containing a | ATCACTTCCAACGATTCATAATGGACACCACACAGGACAGCATGTTGACCAGTTT |
| CR2 portion | GTTGCGGGGTTGTCTGTGACATACAGTTGTGAACCTGGCTATTTGCTCACTGGAA |
| and two FH | AAAAGACAATTAAGTGCTTATCTTCAGGAGACTGGGATGGTGTCATCCCGACAT |
| portions | GCAAAGAGGCCCAGTGTGAACATCCAGGAAAGTTTCCCAATGGGCAGGTAAAG |
| without a | GAACCTCTGAGCCTTCAGGTTGGCACAACTGTGTACTTCTCCTGTAATGAAGGGT |
| linker | ACCAATTACAAGGACAACCCTCTAGTCAGTGTGTAATTGTTGAACAGAAAGCCA |
| sequence | TCTGGACTAAGAAGCCAGTATGTAAAGAAATTCTCGAAGATTGTAAAGGTCCTC |
| | CTCCAAGAGAAAATTCAGAAATTCTCTCAGGCTCGTGGTCAGAACAACTATATC |
| | CAGAAGGCACCCAGGCTACCTACAAATGCCGCCCTGGATACCGAACACTTGGCA |
| | CTATTGTAAAAGTATGCAAGAATGGAAAATGGGTGGCGTCTAACCCATCCAGGA |
| | TATGTCGGAAAAAGCCTTGTGGGCATCCCGGAGACACACCCTTTGGGTCCTTTAG |
| | GCTGGCAGTTGGATCTCAATTTGAGTTTGGTGCAAAGGTTGTTTATACCTGTGAT |
| | GATGGGTATCAACTATTAGGTGAAATTGATTACCGTGAATGTGGTGCAGATGGCT |
| | GGATCAATGATATTCCACTATGTGAAGTTGTGAAGTGTCTACCTGTGACAGAACT |
| | CGAGAATGGAAGAATTGTGAGTGGTGCAGCAGAAACAGACCAGGAATACTATTT |
| | TGGACAGGTGGTGCGGTTTGAATGCAATTCAGGCTTCAAGATTGAAGGACATAA |
| | GGAAATTCATTGCTCAGAAAATGGCCTTTGGAGCAATGAAAAGCCACGATGTGT |
| | GGAAATTCTCTGCACACCACCGCGAGTGGAAAATGGAGATGGTATAAATGTGAA |
| | ACCAGTTTACAAGGAGAATGAAAGATACCACTATAAGTGTAAGCATGGTTATGT |
| | GCCCAAAGAAAGAGGGGATGCCGTCTGCACAGGCTCTGGATGGAGTTCTCAGCC |
| | TTTCTGTGAAGAAAAGAGATGCTCACCTCCTTATATTCTAAATGGTATCTACACA |
| | CCTCACAGGATTATACACAGAAGTGATGATGAAATCAGATATGAATGTAATTAT |
| | GGCTTCTATCCTGTAACTGGATCAACTGTTTCAAAGTGTACACCCACTGGCTGGA |
| | TCCCTGTTCCAAGATGTACCGAAGATTGTAAAGGTCCTCCTCCAAGAGAAAATT |
| | CAGAAATTCTCTCAGGCTCGTGGTCAGAACAACTATATCCAGAAGGCACCCAGG |
| | CTACCTACAAATGCCGCCCTGGATACCGAACACTTGGCACTATTGTAAAAGTAT |
| | GCAAGAATGGAAAATGGGTGGCGTCTAACCCATCCAGGATATGTCGGAAAAAG |
| | CCTTGTGGGCATCCCGGAGACACACCCTTTGGGTCCTTTAGGCTGGCAGTTGGA |
| | TCTCAATTTGAGTTTGGTGCAAAGGTTGTTTATACCTGTGATGATGGGTATCAAC |
| | TATTAGGTGAAATTGATTACCGTGAATGTGGTGCAGATGGCTGGATCAATGATA |
| | TTCCACTATGTGAAGTTGTGAAGTGTCTACCTGTGACAGAACTCGAGAATGGAA |
| | GAATTGTGAGTGGTGCAGCAGAAACAGACCAGGAATACTATTTTGGACAGGTGG |
| | TGCGGTTTGAATGCAATTCAGGCTTCAAGATTGAAGGACATAAGGAAATTCATT |
| | GCTCAGAAAATGGCCTTTGGAGCAATGAAAAGCCACGATGTGTGGAAATTCTCT |
| | GCACACCACCGCGAGTGGAAAATGGAGATGGTATAAATGTGAAACCAGTTTAC |
| | AAGGAGAATGAAAGATACCACTATAAGTGTAAGCATGGTTATGTGCCCAAAGA |
| | AAGAGGGGATGCCGTCTGCACAGGCTCTGGATGGAGTTCTCAGCCTTTCTGTGA |
| | AGAAAAGAGATGCTCACCTCCTTATATTCTAAATGGTATCTACACACCTCACAG |
| | GATTATACACAGAAGTGATGATGAAATCAGATATGAATGTAATTATGGCTTCTA |
| | TCCTGTAACTGGATCAACTGTTTCAAAGTGTACACCCACTGGCTGGATCCCTGTT |
| | CCAAGATGTACCTAA |
| SEQ ID | GAATTCGCCGCCACCATGCCCATGGGGTCTCTGCAACCGCTGGCCACCTTGTAC |
| NO: 20 | CTGCTGGGGATGCTGGTCGCTTCCGTGCTAGCGATTTCTTGTGACCCTCCTCCTG |
| Exemplary | AAGTCAAAAATGCTCGGAAACCCTATTATTCTCTTCCCATAGTTCCTGGAACTG |
| DNA | TTCTGAGGTACACTTGTTCACCTAGCTACCGCCTCATTGGAGAAAAGGCTATC |
| sequence of | TTTTGTATAAGTGAAAATCAAGTGCATGCCACCTGGGATAAAGCTCCTCCTAT |
| CR2LFHFH, a | ATGTGAATCTGTGAATAAAACCATTTCTTGCTCAGATCCCATAGTACCAGGGG |
| mouse CR2- | GATTCATGAATAAAGGATCTAAGGCACCATTCAGACATGGTGATTCTGTGACA |
| FH fusion | TTTACCTGTAAAGCCAACTTCACCATGAAAGGAAGCAAAACTGTCTGGTGCCA |
| protein | GGCAAATGAAATGTGGGGACCAACAGCTCTGCCAGTCTGTGAGAGTGATTTCC |
| containing a | CTCTGGAGTGCCCATCACTTCCAACGATTCATAATGGACACCACACAGGACAG |
| CR2 portion | CATGTTGACCAGTTTGTTGCGGGGTTGTCTGTGACATACAGTTGTGAACCTGGC |
| linked to two | TATTTGCTCACTGGAAAAAAGACAATTAAGTGCTTATCTTCAGGAGACTGGGA |
| FH portions | TGGTGTCATCCCGACATGCAAAGAGGCCCAGTGTGAACATCCAGGAAAGTTTC |
| via a linker | CCAATGGGCAGGTAAAGGAACCTCTGAGCCTTCAGGTTGGCACAACTGTGTAC |
| sequence | TTCTCCTGTAATGAAGGGTACCAATTACAAGGACAACCCTCTAGTCAGTGTGTA |
| | ATTGTTGAACAGAAAGCCATCTGGACTAAGAAGCCAGTATGTAAAGAAATTCT |
| | CGGCGGAGGTGGGTCGGGTGGCGGCGGATCTGAAGATTGTAAAGGTCCTCCTC |
| | CAAGAGAAAATTCAGAAATTCTCTCAGGCTCGTGGTCAGAACAACTATATCCAG |
| | AAGGCACCCAGGCTACCTACAAATGCCGCCCTGGATACCGAACACTTGGCACTA |
| | TTGTAAAAGTATGCAAGAATGGAAAATGGGTGGCGTCTAACCCATCCAGGATAT |
| | GTCGGAAAAAGCCTTGTGGGCATCCCGGAGACACACCCTTTGGGTCCTTTAGGCT |
| | GGCAGTTGGATCTCAATTTGAGTTTGGTGCAAAGGTTGTTTATACCTGTGATGATG |
| | GGTATCAACTATTAGGTGAAATTGATTACCGTGAATGTGGTGCAGATGGCTGGAT |
| | CAATGATATTCCACTATGTGAAGTTGTGAAGTGTCTACCTGTGACAGAACTCGAG |
| | AATGGAAGAATTGTGAGTGGTGCAGCAGAAACAGACCAGGAATACTATTTTGGA |
| | CAGGTGGTGCGGTTTGAATGCAATTCAGGCTTCAAGATTGAAGGACATAAGGAA |
| | ATTCATTGCTCAGAAAATGGCCTTTGGAGCAATGAAAAGCCACGATGTGTGGAA |
| | ATTCTCTGCACACCACCGCGAGTGGAAAATGGAGATGGTATAAATGTGAAACCA |
| | GTTTACAAGGAGAATGAAAGATACCACTATAAGTGTAAGCATGGTTATGTGCCC |
| | AAAGAAAGAGGGGATGCCGTCTGCACAGGCTCTGGATGGAGTTCTCAGCCTTTC |
| | TGTGAAGAAAAGAGATGCTCACCTCCTTATATTCTAAATGGTATCTACACACCTC |
| | ACAGGATTATACACAGAAGTGATGATGAAATCAGATATGAATGTAATTATGGCT |
| | TCTATCCTGTAACTGGATCAACTGTTTCAAAGTGTACACCCACTGGCTGGATCCC |
| | TGTTCCAAGATGTACCGAAGATTGTAAAGGTCCTCCTCCAAGAGAAAATTCAGA |
| | AATTCTCTCAGGCTCGTGGTCAGAACAACTATATCCAGAAGGCACCCAGGCTAC |
| | CTACAAATGCCGCCCTGGATACCGAACACTTGGCACTATTGTAAAAGTATGCAA |
| | GAATGGAAAATGGGTGGCGTCTAACCCATCCAGGATATGTCGGAAAAAGCCTTG |
| | TGGGCATCCCGGAGACACACCCTTTGGGTCCTTTAGGCTGGCAGTTGGATCTCAA |
| | TTTGAGTTTGGTGCAAAGGTTGTTTATACCTGTGATGATGGGTATCAACTATTAG |
| | GTGAAATTGATTACCGTGAATGTGGTGCAGATGGCTGGATCAATGATATTCCACT |
| | ATGTGAAGTTGTGAAGTGTCTACCTGTGACAGAACTCGAGAATGGAAGAATTGT |
| | GAGTGGTGCAGCAGAAACAGACCAGGAATACTATTTTGGACAGGTGGTGCGGTT |
| | TGAATGCAATTCAGGCTTCAAGATTGAAGGACATAAGGAAATTCATTGCTCAGA |
| | AAATGGCCTTTGGAGCAATGAAAAGCCACGATGTGTGGAAATTCTCTGCACACC |
| | ACCGCGAGTGGAAAATGGAGATGGTATAAATGTGAAACCAGTTTACAAGGAGA |
| | ATGAAAGATACCACTATAAGTGTAAGCATGGTTATGTGCCCAAAGAAAGAGGG |
| | GATGCCGTCTGCACAGGCTCTGGATGGAGTTCTCAGCCTTTCTGTGAAGAAAAG |
| | AGATGCTCACCTCCTTATATTCTAAATGGTATCTACACACCTCACAGGATTATAC |
| | ACAGAAGTGATGATGAAATCAGATATGAATGTAATTATGGCTTCTATCCTGTAA |
| | CTGGATCAACTGTTTCAAAGTGTACACCCACTGGCTGGATCCCTGTTCCAAGATG |
| | TACCTAA |
| SEQ ID | ISCGSPPPILNGRISYYSTPIAVGTVIRYSCSGTFRLIGEKSLLCITKDKVDGTWDKPAP |
| NO: 21 | KCEYFNKYSSCPEPIVPGGYKIRGSTPYRHGDSVTFACKTNFSMNGNKSVWCQANN |
| Human CR2- | MWGPTRLPTCVSVFPLECPALPMIHNGHHTSENVGSIAPGLSVTYSCESGYLLVGEK |
| FH amino acid | IINCLSSGKWSAVPPTCEEARCKSLGRFPNGKVKEPPILRVGVTANFFCDEGYRLQGP |
| sequence | PSSRCVIAGQGVAWTKMPVCEEIFEDCNELPPRRNTEILTGSWSDQTYPEGTQAIYK |
| | CRPGYRSLGNVIMVCRKGEWVALNPLRKCQKRPCGHPGDTPFGTFTLTGGNVFEY |
| | GVKAVYTCNEGYQLLGEINYRECDTDGWTNDIPICEVVKCLPVTAPENGKIVSSAM |
| | EPDREYHFGQAVRFVCNSGYKIEGDEEMHCSDDGFWSKEKPKCVEISCKSPDVING |
| | SPISQKIIYKENERFQYKCNMGYEYSERGDAVCTESGWRPLPSCEEKSCDNPYIPNG |
| | DYSPLRIKHRTGDEITYQCRNGFYPATRGNTAKCTSTGWIPAPRCTLK |
| SEQ ID | GCCGCcaCCATGGGAGCCGCTGGTCTGCTCGGCGTGTTCCTCGCCTTGGTGGCA |
| NO: 22 | CCTGGCGTCCTGGGCATCAGCTGCGGTTCCCCTCCACCAATCCTGAATGGCAG |
| Human CR2- | AATCTCCTATTACTCCACACCAATCGCCGTCGGCACTGTGATCAGATACAGCT |
| FH DNA | GTTCAGGGACTTTTCGGCTGATCGGCGAGAAAAGCCTCCTCTGCATTACCAAG |
| sequence | GATAAGGTCGATGGGACATGGGATAAACCAGCTCCTAAGTGCGAGTACTTCA |
| (including | ATAAGTATAGTTCATGTCCAGAGCCCATTGTTCCTGGTGGCTACAAGATTCGG |
| signal peptide) | GGGAGCACACCCTATCGCCACGGTGACTCAGTGACCTTTGCTTGTAAAACCAA |
| | CTTCTCAATGAACGGTAATAAGTCAGTGTGGTGTCAGGCCAATAATATGTGGG |
| | GTCCTACACGACTCCCCACCTGTGTGTCCGTGTTCCCCTTGGAATGCCCCGCCC |
| | TGCCCATGATCCATAATGGACACCACACCAGCGAGAATGTCGGGAGTATCGCA |
| | CCTGGATTGAGTGTCACCTACTCATGCGAGTCTGGCTACCTGCTTGTAGGTGAA |
| | AAAATTATTAATTGCTTGTCCTCCGGCAAATGGAGTGCCGTTCCCCCAACTTGT |
| | GAAGAGGCCCGGTGCAAATCCCTCGGCCGCTTCCCTAATGGTAAAGTTAAAGA |
| | GCCTCCAATCCTCAGAGTGGGGGTGACCGCTAACTTCTTCTGTGATGAAGGCTA |
| | CCGGTTGCAGGGACCACCCAGTAGCCGGTGTGTCATAGCTGGGCAGGGAGTGG |
| | CTTGGACAAAGATGCCCGTTTGTGAGGAAATCTTCGAAGACTGTAATGAGCTG |
| | CCCCCAAGACGGAATACAGAGATCCTCACAGGCTCTTGGTCCGATCAAACTTA |
| | TCCAGAGGGTACCCAGGCAATTTACAAGTGCAGACCTGGATACAGGAGCCTGG |
| | GCAATGTGATTATGGTGTGCCGCAAGGGGGAGTGGGTGGCCCTTAATCCTCTC |
| | CGGAAGTGTCAGAAAAGACCATGCGGACACCCTGGAGATACACCTTTCGGTAC |
| | CTTTACCCTTACCGGCGGCAATGTCTTCGAGTATGGCGTCAAGGCCGTGTACAC |
| | TTGTAACGAGGGATACCAGCTGCTGGGGGAAATAAACTATCGTGAGTGTGACA |
| | CTGACGGGTGGACTAACGACATCCCCATTTGCGAGGTGGTCAAGTGCCTTCCTG |
| | TAACCGCTCCCGAAAATGGTAAGATCGTATCTTCCGCAATGGAGCCTGaTCGGG |
| | AATACcaCTTTGGACAAGCCGTTCGGTTCGTATGTAATTCAGGGTATAAAATTGA |
| | GGGCGATGAGGAGATGCACTGCAGTGATGACGGCTTTTGGTCAAAGGAAAAGC |
| | CAAAGTGCGTAGAGATCAGTTGTAAGTCTCCTGACGTTATTAACGGGAGTCCCA |
| | TCAGTCAGAAGATCATTTACAAGGAAAACGAGAGGTTCCAGTATAAATGCAATA |
| | TGGGATATGAGTACTCCGAAAGAGGGGACGCCGTGTGCACAGAGTCCGGATGGC |
| | GACCTTTGCCATCTTGTGAAGAAAAGTCTTGTGACAACCCCTATATTCCTAACGG |
| | AGATTACTCTCCTCTGCGCATCAAGCACCGAACTGGGGACGAGATCACTTACCAA |
| | TGTCGAAACGGCTTCTACCCTGCTACCAGAGGTAACACTGCCAAGTGTACCAGCA |
| | CCGGTTGGATTCCCGCCCCCAGATGCACACTTAAATGATAA |
| SEQ ID | ISCGSPPPILNGRISYYSTPIAVGTVIRYSCSGTFRLIGEKSLLCITKDKVDGTWDKPA |
| NO: 23 | PKCEYFNKYSSCPEPIVPGGYKIRGSTPYRHGDSVTFACKTNFSMNGNKSVWCQAN |
| Human CR2- | NMWGPTRLPTCVSVFPLECPALPMIHNGHHTSENVGSIAPGLSVTYSCESGYLLVGE |
| FH2 amino | KIINCLSSGKWSAVPPTCEEARCKSLGRFPNGKVKEPPILRVGVTANFFCDEGYRLQ |
| acid sequence | GPPSSRCVIAGQGVAWTKMPVCEEIFEDCNELPPRRNTEILTGSWSDQTYPEGTQAI |
| | YKCRPGYRSLGNVIMVCRKGEWVALNPLRKCQKRPCGHPGDTPFGTFTLTGGNVF |
| | EYGVKAVYTCNEGYQLLGEINYRECDTDGWTNDIPICEVVKCLPVTAPENGKIVSS |
| | AMEPDREYHFGQAVRFVCNSGYKIEGDEEMHCSDDGFWSKEKPKCVEISCKSPDVI |
| | NGSPISQKIIYKENERFQYKCNMGYEYSERGDAVCTESGWRPLPSCEEKSCDNPYIP |
| | NGDYSPLRIKHRTGDEITYQCRNGFYPATRGNTAKCTSTGWIPAPRCTEDCNELPPR |
| | RNTEILTGSWSDQTYPEGTQAIYKCRPGYRSLGNVIMVCRKGEWVALNPLRKCQKR |
| | PCGHPGDTPFGTFTLTGGNVFEYGVKAVYTCNEGYQLLGEINYRECDTDGWTNDIP |
| | ICEVVKCLPVTAPENGKIVSSAMEPDREYHFGQAVRFVCNSGYKIEGDEEMHCSDD |
| | GFWSKEKPKCVEISCKSPDVINGSPISQKIIYKENERFQYKCNMGYEYSERGDAVCT |
| | ESGWRPLPSCEEKSCDNPYIPNGDYSPLRIKHRTGDEITYQCRNGFYPATRGNTAKC |
| | TSTGWIPAPRCTLK |
| SEQ ID | CGCCGCCACCATGGGCGCAGCAGGCTTGTTGGGCGTGTTCCTGGCATTGGTGG |
| NO: 24 | CACCCGGCGTATTGGGCATTTCATGCGGCTCTCCTCCACCCATTCTCAATGGA |
| Human CR2- | AGGATCTCCTACTACAGCACCCCCATAGCTGTCGGCACCGTTATCCGATACAG |
| FH2 DNA | TTGTTCCGGTACTTTCCGGCTTATCGGCGAAAAGTCTTTGCTGTGCATTACCAA |
| sequence | GGATAAAGTGGACGGGACTTGGGACAAACCCGCACCTAAGTGCGAGTATTTT |
| (including | AACAAATATAGCAGCTGCCCTGAGCCTATAGTACCCGGGGGGTATAAAATCC |
| signal peptide) | GGGGCTCTACTCCCTATCGTCATGGCGATTCTGTGACCTTCGCATGTAAAACT |
| | AATTTTTCAATGAATGGCAACAAGTCTGTATGGTGTCAAGCAAATAACATGT |
| | GGGGACCTACCCGCCTGCCAACCTGTGTGTCAGTGTTTCCCCTGGAATGTCCA |
| | GCCCTCCCTATGATCCACAACGGACATCACACCAGCGAAAACGTTGGATCCA |
| | TCGCACCAGGGCTCTCTGTGACTTACTCTTGCGAGTCCGGGTACCTGCTCGTG |
| | GGTGAAAAGATCATCAACTGCCTCAGTAGTGGTAAATGGTCCGCCGTGCCTC |
| | CCACATGTGAAGAGGCCCGGTGCAAGAGCCTGGGCCGGTTCCCCAACGGAA |
| | AAGTGAAGGAACCTCCTATCTTGAGGGTTGGTGTGACCGCTAACTTTTTCTGC |
| | GACGAGGGGTACAGGCTCCAAGGGCCTCCCTCTAGTCGGTGCGTAATCGCCG |
| | GTCAAGGAGTCGCATGGACTAAGATGCCTGTGTGTGAGGAGATTTTCGAGGA |
| | TTGTAATGAATTGCCACCCAGGAGAAATACTGAAATCCTGACAGGCTCTTGGT |
| | CTGATCAGACTTATCCAGAAGGCACCCAGGCCATTTACAAGTGTCGGCCTGGA |
| | TACAGATCTCTGGGAAATGTGATCATGGTATGTAGGAAAGGAGAGTGGGTGG |
| | CTTTGAACCCCCTCCGCAAGTGTCAGAAAAGACCATGCGGGCATCCTGGAGA |
| | CACCCCATTCGGGACATTTACACTGACAGGCGGAAACGTATTTGAGTACGGA |
| | GTCAAGGCCGTTTATACATGTAACGAAGGGTATCAACTGCTGGGAGAAATCA |
| | ACTATAGGGAGTGCGACACTGACGGATGGACAAACGACATTCCAATCTGCGA |
| | AGTGGTGAAATGTCTTCCAGTTACAGCCCCTGAAAACGGGAAAATCGTGTCCT |
| | CCGCTATGGAGCCTGACCGGGAATATCATTTCGGCCAGGCCGTTAGATTCGTG |
| | TGTAATAGCGGCTACAAAATCGAGGGCGACGAAGAAATGCATTGCAGCGATG |
| | ACGGGTTCTGGAGCAAGGAGAAGCCTAAATGCGTCGAAATTTCATGCAAGAGT |
| | CCCGACGTCATAAACGGTTCTCCAATTTCCCAGAAGATCATTTATAAGGAGAAT |
| | GAGCGGTTCCAGTATAAGTGTAATATGGGCTACGAGTACAGCGAACGCGGTGA |
| | CGCCGTGTGTACCGAAAGTGGCTGGAGACCACTGCCTAGTTGCGAGGAGAAATC |
| | CTGCGACAACCCTTATATTCCCAACGGGGACTACTCTCCTCTGAGAATCAAGCAT |
| | CGGACTGGCGACGAGATTACTTACCAATGCAGGAACGGATTCTATCCAGCAACT |
| | CGGGGCAATACCGCTAAGTGTACCTCCACAGGCTGGATACCCGCTCCTAGATGTA |
| | CAGAGGACTGCAATGAACTGCCACCTCGGCGCAATACAGAAATTTTGACTGGAT |
| | CATGGTCTGACCAGACTTACCCCGAGGGCACCCAGGCCATCTACAAATGTAGGC |
| | CCGGTTATCGAAGTTTGGGTAACGTGATTATGGTGTGTCGAAAAGGTGAATGGG |
| | TAGCACTCAATCCCCTCCGTAAATGCCAGAAGCGTCCTTGTGGGCACCCAGGCG |
| | ATACCCCTTTTGGAACTTTCACCCTGACTGGAGGAAACGTCTTTGAATATGGTGT |
| | GAAAGCCGTGTACACATGCAATGAAGGGTACCAACTGCTCGGAGAGATAAACTA |
| | TCGGGAGTGCGATACAGATGGATGGACCAATGATATACCAATCTGCGAGGTGGT |
| | GAAGTGTCTCCCAGTCACCGCTCCTGAGAACGGAAAGATCGTCAGTTCTGCTATG |
| | GAACCTGACAGGGAATACCACTTTGGGCAAGCCGTCCGCTTCGTGTGCAATTCAG |
| | GGTACAAGATAGAAGGCGACGAAGAGATGCACTGTTCCGACGATGGTTTCTGGT |
| | CTAAGGAGAAGCCTAAATGTGTCGAGATTAGCTGCAAGTCTCCCGATGTTATTAA |
| | CGGCTCTCCCATCTCTCAAAAAATTATTTATAAGGAAAACGAAAGATTTCAGTAC |
| | AAGTGCAATATGGGTTATGAGTACAGTGAACGTGGAGACGCCGTGTGCACAGAG |
| | TCCGGGTGGCGTCCACTGCCCAGCTGCGAAGAAAAATCCTGTGACAACCCCTACA |
| | TCCCCAATGGCGACTATTCCCCCCTGCGCATCAAACATCGTACTGGCGATGAAATT |
| | ACTTACCAGTGCCGCAACGGGTTCTACCCTGCCACCCGGGGTAACACAGCCAAAT |
| | GCACCTCCACCGGATGGATCCCCGCCCCACGCTGTACCTTGAAATGATGA |
| SEQ ID | MGAAGLLGVFLALVAPGVLG |
| NO: 25 | |
| CR2 peptide | |
| sequence | |
| SEQ ID | ATGGGAGCCGCTGGTCTGCTCGGCGTGTTCCTCGCCTTGGTGGCACCT |
| NO: 26 | GGCGTCCTGGGC |
| CR2 | |
| nucleotide | |
| sequence | |
| SEQ ID | DIQMTQSPSSLSASVGDRVTITCGASENIYGALNWYQQKPGKAPKLLI |
| NO: 27 | YGATNLADGVPSRFSGSGSGTDFTLTISSLQPEDFATYYCQNVLNTPLTF |
| Ec SCFV (no | GQGTKVEIKRTGGGGSGGGGSGGGGSQVQLVQSGAEVKKPGASVKVSCKA |
| n-terminal | SGYIFSNYWIQWVRQAPGQGLEWMGEILPGSGSTEYTENFKDRVTMTRDT |
| Ala) - Amino | STSTVYMELSSLRSEDTAVYYCARYFFGSSPNWYFDVWGQGTLVTVSS |
| Acid | |
| SEQ ID NO: 28 | GATATCCAGATGACCCAGTCCCCGTCCTCCCTGTCCGCCTCTGTGGGCGAT |
| Ec SCFV | AGGGTCACCATCACCTGCGGCGCCAGCGAAAACATCTATGGCGCGCTGAA |
| nucleic acid | CTGGTATCAACAGAAACCCGGGAAAGCTCCGAAGCTTCTGATTTACGGTG |
| | CGACGAACCTGGCAGATGGAGTCCCTTCTCGCTTCTCTGGATCCGGCTCCG |
| | GAACGGATTTCACTCTGACCATCAGCAGTCTGCAGCCTGAAGACTTCGCTA |
| | CGTATTACTGTCAGAACGTTTTAAATACTCCGTTGACTTTCGGACAGGGTA |
| | CCAAGGTGGAAATAAAACGTACTGGCGGTGGTGGTTCTGGTGGCGGTGGA |
| | TCTGGTGGTGGCGGTTCTCAAGTCCAACTGGTGCAATCCGGCGCCGAGGTC |
| | AAGAAGCCAGGGGCCTCAGTCAAAGTGTCCTGTAAAGCTAGCGGCTATATT |
| | TTTTCTAATTATTGGATTCAATGGGTGCGTCAGGCCCCCGGGCAGGGCCTGG |
| | AATGGATGGGTGAGATCTTACCGGGCTCTGGTAGCACCGAATATACCGAAA |
| | ATTTTAAAGACCGTGTTACTATGACGCGTGACACTTCGACTAGTACAGTATA |
| | CATGGAGCTCTCCAGCCTGCGATCGGAGGACACGGCCGTCTATTATTGCGCG |
| | CGTTATTTTTTTGGTTCTAGCCCGAATTGGTATTTTGATGTTTGGGGTCAAGG |
| | AACCCTGGTCACTGTCTCGAGCTG |
| SEQ ID NO: 29 | ADIQMTQSPSSLSASVGDRVTITCGASENIYGALNWYQRKPGKAPKLLI |
| Pex | YGATNLADGVPSRFSGSGSGTDFTLTISSLQPEDFATYYCQNVLNTPLTF |
| (variant of EC) | GQGTKVEIKRTGGGGSGGGGSGGGGSQVQLVQSGAEVKKPGASVKVSCKA |
| | SGYIFSNYWIQWVRQAPGQGLEWMGEILPGSGSTEYTENFKDRVTMTRDT |
| | STSTVYMELSSLRSEDTAVYYCARYFFGSSPNWYFDVWGQGTLVTVSS |
| SEQ ID NO: 30 | QVQLVQSGAEVKKPGASVKVSCKASGYIFSNYWIQ |
| (heavy chain | WVRQAPGQGLEWMGEILPGSGSTEYTENFKDRVTM |
| amino acid | TRDTSTSTVYMELSSLRSEDTAVYYCARYFFGSSPNW |
| sequence for | YFDVWGQGTLVTVSSASTKGPSVFPLAPCSRSTSESTAA |
| EC) | LGCLVKDYFPEPVTVSWNSGALTSGVHTFPAVLQSSGLYS |
| | LSSVVTVPSSNFGTQTYTCNVDHKPSNTKVDKTVERKCCV |
| | ECPPCPAPPVAGPSVFLFPPKPKDTLMISRTPEVTCVVVD |
| | VSQEDPEVQFNWYVDGVEVHNAKTKPREEQFNSTYRVVS |
| | VLTVLHQDWLNGKEYKCKVSNKGLPSSIEKTISKAKGQPR |
| | EPQVYTLPPSQEEMTKNQVSLTCLVKGFYPSDIAVEWESN |
| | GQPENNYKTTPPVLDSDGSFFLYSRLTVDKSRWQEGNVFS |
| | CSVMHEALHNHYTQKSLSLSLGK |
| SEQ ID NO: 31 | DIQMTQSPSSLSASVGDRVTITCGASENIYGALNWYQQKPG |
| (light chain | KAPKLLIYGATNLADGVPSRFSGSGSGTDFTLTISSLQPEDF |
| amino acid | ATYYCQNVLNTPLTFGQGTKVEIKRTVAAPSVFIFPPSDEQL |
| sequence for | KSGTASVVCLLNNFYPREAKVQWKVDNALQSGNSQESVTEQD |
| EC) | SKDSTYSLSSTLTLSKADYEKHKVYACEVTHQGLSSPVTKSFNR |
| | GEC |

1. A method to prolong survival of an organ that is transplanted from a donor mammal to a recipient mammal, wherein the method comprises administering a complement inhibitor to the organ prior to transplantation, and wherein the complement inhibitor has a maximum molecular weight of 70 kDa and/or a half-life shorter than 10 days.

2. A method to prolong survival of an organ that is transplanted from a donor mammal to a recipient mammal, wherein the method comprises administering a complement inhibitor to the organ prior to transplantation, wherein the complement inhibitor is a human CR2-FH fusion protein comprising SEQ ID NO: 3 or a single chain antibody comprising SEQ ID NO:27 or SEQ ID NO:29.

3. A method to prevent or attenuate rejection of a transplanted organ in a recipient mammal, wherein the method comprises administering a complement inhibitor to the organ prior to transplantation, and wherein the complement inhibitor has a maximum molecular weight of 70 kDa and/or a half-life shorter than 10 days.

4. A method to prevent or attenuate rejection of a transplanted organ in a recipient mammal, wherein the method comprises administering a complement inhibitor to the organ prior to transplantation, wherein the complement inhibitor is a human CR2-FH fusion protein comprising SEQ ID NO:3 or a single chain antibody comprising SEQ ID NO:27 or SEQ ID NO:29.

5. The method of claim 3, wherein the rejection is hyperacute rejection, antibody-mediated rejection (AMR), or chronic rejection.

6. The method of claim 1, wherein the complement inhibitor has a molecular weight of about 26 kDa or about 65 kDa.

7. (canceled)

8. (canceled)

9. The method of claim 1, wherein the recipient mammal is not vaccinated against Neisseria meningitides prior to transplantation.

10. The method of claim 1, wherein the complement inhibitor has substantially cleared from the organ prior to transplantation into the recipient mammal.

11. The method of claim 1, wherein the complement inhibitor is a human CR2-FH fusion protein comprising SEQ ID NO: 3.

12. The method of claim 1, wherein the complement inhibitor is a single chain antibody.

13. The method of claim 12, wherein the complement inhibitor is a single chain anti-05 antibody.

14. The method of claim 13, wherein the complement inhibitor is a single chain anti-C5 antibody comprising SEQ ID NO:27 or SEQ ID NO:29.

15. The method of claim 1, wherein the organ is selected from the group consisting of: kidney, heart, lung, pancreas, liver, vascular tissue, eye, cornea, lens, skin, bone marrow, muscle, connective tissue, gastrointestinal tissue, nervous tissue, bone, stem cells, islets, cartilage, hepatocytes, and hematopoietic cells.

16. The method of claim 1, wherein the complement inhibitor is administered to the organ after removal of the organ from a donor mammal and before transplant of the organ into a recipient mammal.

17. The method of claim 1, wherein the complement inhibitor is administered at an organ procurement center.

18. The method of claim 1, wherein the complement inhibitor is administered immediately prior to transplantation.

19. The method of claim 1, wherein the donor mammal and recipient mammals are humans.

20. The method of claim 1, wherein the recipient is not treated with a complement inhibitor after transplantation.

21. The method of claim 1, wherein administering the complement inhibitor to the organ comprises (i) perfusing the organ with a solution comprising the complement inhibitor or (ii) soaking the organ in a solution comprising the complement inhibitor.

22. (canceled)

23. The method of claim 21, wherein the organ is perfused or soaked for 0.5 to 60 hours.

24. (canceled)

25. (canceled)


SEQUENCE LISTING (42 sequences)

SEQ ID NO: 1 - protein, 1087 aa, Homo sapiens [sequence omitted]

SEQ ID NO: 2 - protein, 1231 aa, Homo sapiens
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNHGRKFVQGKSIDVACHPGYALPKAQTTVTCMENGWSPTPRCIRVKTCSKSSIDIENGFISESQYTYALKEKAKYQCKLGYVTADGETSGSIRCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 3 - protein, 570 aa, Artificial Sequence
  source: /note=“Description of Artificial Sequence Synthetic polypeptide”
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNINNMWGPTRLPTCVSVFPLECPALPMIHNGHHTSENVGSIAPGLSVTYSCESGYLLVGEKIINCLSSGKWSAVPPTCEEAXCKSLGRFPNGKVKEPPILRVGVTANFFCDEGYRLQGPPSSRCVIAGQGVAWTKMPVCGGGGSGGGGSCVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCT

SEQ ID NO: 4 - DNA, 1711 bp, Artificial Sequence
  source: /note=“Description of Artificial Sequence Synthetic polynucleotide”
atttcttgtggctctcctccgcctatcctaaatggccggattagttattattctacccccattgctgttggtaccgtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaacggaaacaagtctgtttggtgtcaagcaaataatataaataatatgtgggggccgacacgactaccaacctgtgtaagtgttttccctctcgagtgtccagcacttcctatgatccacaatggacatcacacaagtgagaatgttggctccattgctccaggattgtctgtgacttacagctgtgaatctggttacttgcttgttggagaaaagatcattaactgtttgtcttcgggaaaatggagtgctgtcccccccacatgtgaagaggcacsctgtaaatctctaggacgatttcccaatgggaaggtaaaggagcctccaattctccgggttggtgtaactgcaaactttttctgtgatgaagggtatcgactgcaaggcccaccttctagtcggtgtgtaattgctggacagggagttgcttggaccaaaatgccagtatgtggcggaggtgggtcgggtggcggcggatcttgtgtagcagaagattgcaatgaacttcctccaagaagaaatacagaaattctgacaggttcctggtctgaccaaacatatccagaaggcacccaggctatctataaatgccgccctggatatagatctcttggaaatgtaataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagccaaatgcacaagtactggctggatacctgctccgagatgtacct

SEQ ID NO: 5 - protein, 560 aa, Artificial Sequence
  source: /note=“Description of Artificial Sequence Synthetic polypeptide”
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCXXXCVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCT

SEQ ID NO: 6 - protein, 560 aa, Artificial Sequence
  source: /note=“Description of Artificial Sequence Synthetic polypeptide”
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCXXXCVAEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCT

SEQ ID NO: 7 - protein, 560 aa, Artificial Sequence
  source: /note=“Description of Artificial Sequence Synthetic polypeptide”
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNINNMWGPTRLPTCVSVFPLECPALPMIHNGHHTSENVGSIAPGLSVTYSCESGYLLVGEKIINCLSSGKWSAVPPTCEEAXCKSLGRFPNGKVKEPPILRVGVTANFFCDEGYRLQGPPSSRCVIAGQGVAWTKMPVCXXXEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCT

SEQ ID NO: 8 - protein, 560 aa, Artificial Sequence
  source: /note=“Description of Artificial Sequence Synthetic polypeptide”
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNINNMWGPTRLPTCVSVFPLECPALPMIHNGHHTSENVGSIAPGLSVTYSCESGYLLVGEKIINCLSSGKWSAVPPTCEEAXCKSLGRFPNGKVKEPPILRVGVTANFFCDEGYRLQGPPSSRCVIAGQGVAWTKMPVCXXXEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCT

SEQ ID NO: 9 - protein, 557 aa, Artificial Sequence
  source: /note=“Description of Artificial Sequence Synthetic polypeptide”
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCXXXEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCT

SEQ ID NO: 10 - protein, 557 aa, Artificial Sequence
  source: /note=“Description of Artificial Sequence Synthetic polypeptide”
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCXXXEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCT

SEQ ID NO: 11 - protein, 21 aa, Artificial Sequence
  source: /note=“Description of Artificial Sequence Synthetic peptide”
MPMGSLQPLATLYLLGMLVAS

SEQ ID NO: 12 - DNA, 72 bp, Artificial Sequence
  source: /note=“Description of Artificial Sequence Synthetic oligonucleotide”
atgcccatggggtctctgcaaccgctggccaccttgtacctgctggggatgctggtcgcttcctgcctcgga

SEQ ID NO: 13 - protein, 17 aa, Artificial Sequence
  source: /note=“Description of Artificial Sequence Synthetic peptide”
MGAAGLLGVFLALVAPG

SEQ ID NO: 14 - DNA, 60 bp, Artificial Sequence
  source: /note=“Description of Artificial Sequence Synthetic oligonucleotide”
atgggcgccgcgggcctgctcggggttttcttggctctcgtcgcaccgggggtcctcggg

SEQ ID NO: 15 - protein, 1025 aa, Mus sp.
MLTWFLFYFSEISCDPPPEVKNARKPYYSLPIVPGTVLRYTCSPSYRLIGEKAIFCISENQVHATWDKAPPICESVNKTISCSDPIVPGGFMNKGSKAPFRHGDSVTFTCKANFTMKGSKTVWCQANEMWGPTALPVCESDFPLECPSLPTIHNGHHTGQHVDQFVAGLSVTYSCEPGYLLTGKKTIKCLSSGDWDGVIPTCKEAQCEHPGKFPNGQVKEPLSLQVGTTVYFSCNEGYQLQGQPSSQCVIVEQKAIWTKKPVCKEILCPPPPPVRNGSHTGSFSENVPYGSTVTYTCDPSPEKGVSFTLIGEKTINCTTGSQKTGIWSGPAPYCVLSTSAVLCLQPKIKRGQILSILKDSYSYNDTVAFSCEPGFTLKGNRSIRCNAHGTWEPPVPVCEKGCQAPPKIINGQKEDSYLLNFDPGTSIRYSCDPGYLLVGEDTIHCTPEGKWTPITPQCTVAECKPVGPHLFKRPQNQFIRTAVNSSCDEGFQLSESAYQLCQGTIPWFIEIRLCKEITCPPPPVIHNGTHTWSSSEDVPYGTVVTYMCYPGPEEGVKFKLIGEQTIHCTSDSRGRGSWSSPAPLCKLSLPAVQCTDVHVENGVKLTDNKAPYFYNDSVMFKCDDGYILSGSSQIRCKANNTWDPEKPLCKKEGCEPMRVHGLPDDSHIKLVKRTCQNGYQLTGYTYEKCQNAENGTWFKKIEVCTVILCQPPPKIANGGHTGMMAKHFLYGNEVSYECDEGFYLLGEKSLQCVNDSKGHGSWSGPPPQCLQSSPLTHCPDPEVKHGYKLNKTHSAFSHNDIVHFVCNQGFIMNGSHLIRCHTNNTWLPGVPTCIRKASLGCQSPSTIPNGNHTGGSIARFPPGMSVMYSCYQGFLMAGEARLICTHEGTWSQPPPFCKEVNCSFPEDTNGIQKGFQPGKTYRFGATVTLECEDGYTLEGSPQSQCQDDSQWNPPLALCKYRRWSTIPLICGISVGSALIILMSVGFCMILKHRESNYYTKTRPKEGALHLETREVYSIDPYNPAS

SEQ ID NO: 16 - protein, 1249 aa, Mus sp.
MRLSARIIWLILWTVCAAEDCKGPPPRENSEILSGSWSEQLYPEGTQATYKCRPGYRTLGTIVKVCKNGKWVASNPSRICRKKPCGHPGDTPFGSFRLAVGSQFEFGAKVVYTCDDGYQLLGEIDYRECGADGWINDIPLCEVVKCLPVTELENGRIVSGAAETDQEYYFGQVVRFECNSGFKIEGHKEIHCSENGLWSNEKPRCVEILCTPPRVENGDGINVKPVYKENERYHYKCKHGYVPKERGDAVCTGSGWSSQPFCEEKRCSPPYILNGIYTPHRIIHRSDDEIRYECNYGFYPVTGSTVSKCTPTGWIPVPRCTLKPCEFPQFKYGRLYYEESLRPNFPVSIGNKYSYKCDNGFSPPSGYSWDYLRCTAQGWEPEVPCVRKCVFHYVENGDSAYWEKVYVQGQSLKVQCYNGYSLQNGQDTMTCTENGWSPPPKCIRIKTCSASDIHIDNGFLSESSSIYALNRETSYRCKQGYVTNTGEISGSITCLQNGWSPQPSCIKSCDMPVFENSITKNTRTWFKLNDKLDYECLVGFENEYKHTKGSITCTYYGWSDTPSCYERECSVPTLDRKLVVSPRKEKYRVGDLLEFSCHSGHRVGPDSVQCYHFGWSPGFPTCKGQVASCAPPLEILNGEINGAKKVEYSHGEVVKYDCKPRFLLKGPNKIQCVDGNWTTLPVCIEEERTCGDIPELEHGSAKCSVPPYHHGDSVEFICEENFTMIGHGSVSCISGKWTQLPKCVATDQLEKCRVLKSTGIEAIKPKLTEFTHNSTMDYKCRDKQEYERSICINGKWDPEPNCTSKTSCPPPPQIPNTQVIETTVKYLDGEKLSVLCQDNYLTQDSEEMVCKDGRWQSLPRCIEKIPCSQPPTIEHGSINLPRSSEERRDSIESSSHEHGTTFSYVCDDGFRIPEENRITCYMGKWSTPPRCVGLPCGPPPSIPLGTVSLELESYQHGEEVTYHCSTGFGIDGPAFIICEGGKWSDPPKCIKTDCDVLPTVKNAIIRGKSKKSYRTGEQVTFRCQSPYQMNGSDTVTCVNSRWIGQPVCKDNSCVDPPHVPNATIVTRTKNKYLHGDRVRYECNKPLELFGQVEVMCENGIWTEKPKCRGLFDLSLKPSNVFSLDSTGKCGPPPPIDNGDITSLSLPVYEPLSSVEYQCQKYYLLKGKKTITCTNGKWSEPPTCLHACVIPENIMESHNIILKWRHTEKIYSHSGEDIEFGCKYGYYKARDSPPFRTKCINGTINYPTCV

SEQ ID NO: 17 - protein, 559 aa, Artificial Sequence
  source: /note=“Description of Artificial Sequence Synthetic polypeptide”
ISCDPPPEVKNARKPYYSLPIVPGTVLRYTCSPSYRLIGEKAIFCISENQVHATWDKAPPICESVNKTISCSDPIVPGGFMNKGSKAPFRHGDSVTFTCKANFTMKGSKTVWCQANEMWGPTALPVCESDFPLECPSLPTIHNGHHTGQHVDQFVAGLSVTYSCEPGYLLTGKKTIKCLSSGDWDGVIPTCKEAQCEHPGKFPNGQVKEPLSLQVGTTVYFSCNEGYQLQGQPSSQCVIVEQKAIWTKKPVCKEILEDCKGPPPRENSEILSGSWSEQLYPEGTQATYKCRPGYRTLGTIVKVCKNGKWVASNPSRICRKKPCGHPGDTPFGSFRLAVGSQFEFGAKVVYTCDDGYQLLGEIDYRECGADGWINDIPLCEVVKCLPVTELENGRIVSGAAETDQEYYFGQVVRFECNSGFKIEGHKEIHCSENGLWSNEKPRCVEILCTPPRVENGDGINVKPVYKENERYHYKCKHGYVPKERGDAVCTGSGWSSQPFCEEKRCSPPYILNGIYTPHRIIHRSDDEIRYECNYGFYPVTGSTVSKCTPTGWIPVPRCT

SEQ ID NO: 18 - DNA, 1750 bp, Artificial Sequence
  source: /note=“Description of Artificial Sequence Synthetic polynucleotide”
atgcccatggggtctctgcaaccgctggccaccttgtacctgctggggatgctggtcgcttccgtgctagcgatttcttgtgaccctcctcctgaagtcaaaaatgctcggaaaccctattattctcttcccatagttcctggaactgttctgaggtacacttgttcacctagctaccgcctcattggagaaaaggctatcttttgtataagtgaaaatcaagtgcatgccacctgggataaagctcctcctatatgtgaatctgtgaataaaaccatttcttgctcagatcccatagtaccagggggattcatgaataaaggatctaaggcaccattcagacatggtgattctgtgacatttacctgtaaagccaacttcaccatgaaaggaagcaaaactgtctggtgccaggcaaatgaaatgtggggaccaacagctctgccagtctgtgagagtgatttccctctggagtgcccatcacttccaacgattcataatggacaccacacaggacagcatgttgaccagtttgttgcggggttgtctgtgacatacagttgtgaacctggctatttgctcactggaaaaaagacaattaagtgcttatcttcaggagactgggatggtgtcatcccgacatgcaaagaggcccagtgtgaacatccaggaaagtttcccaatgggcaggtaaaggaacctctgagccttcaggttggcacaactgtgtacttctcctgtaatgaagggtaccaattacaaggacaaccctctagtcagtgtgtaattgttgaacagaaagccatctggactaagaagccagtatgtaaagaaattctcgaagattgtaaaggtcctcctccaagagaaaattcagaaattctctcaggctcgtggtcagaacaactatatccagaaggcacccaggctacctacaaatgccgccctggataccgaacacttggcactattgtaaaagtatgcaagaatggaaaatgggtggcgtctaacccatccaggatatgtcggaaaaagccttgtgggcatcccggagacacaccctttgggtcctttaggctggcagttggatctcaatttgagtttggtgcaaaggttgtttatacctgtgatgatgggtatcaactattaggtgaaattgattaccgtgaatgtggtgcagatggctggatcaatgatattccactatgtgaagttgtgaagtgtctacctgtgacagaactcgagaatggaagaattgtgagtggtgcagcagaaacagaccaggaatactattttggacaggtggtgcggtttgaatgcaattcaggcttcaagattgaaggacataaggaaattcattgctcagaaaatggcctttggagcaatgaaaagccacgatgtgtggaaattctctgcacaccaccgcgagtggaaaatggagatggtataaatgtgaaaccagtttacaaggagaatgaaagataccactataagtgtaagcatggttatgtgcccaaagaaagaggggatgccgtctgcacaggctctggatggagttctcagcctttctgtgaagaaaagagatgctcacctccttatattctaaatggtatctacacacctcacaggattatacacagaagtgatgatgaaatcagatatgaatgtaattatggcttctatcctgtaactggatcaactgtttcaaagtgtacacccactggctggatccctgttccaagatgtacct

SEQ ID NO: 19 - DNA, 2676 bp, Artificial Sequence
  source: /note=“Description of Artificial Sequence Synthetic polynucleotide”
gaattcgccgccaccatgcccatggggtctctgcaaccgctggccaccttgtacctgctggggatgctggtcgcttccgtgctagcgatttcttgtgaccctcctcctgaagtcaaaaatgctcggaaaccctattattctcttcccatagttcctggaactgttctgaggtacacttgttcacctagctaccgcctcattggagaaaaggctatcttttgtataagtgaaaatcaagtgcatgccacctgggataaagctcctcctatatgtgaatctgtgaataaaaccatttcttgctcagatcccatagtaccagggggattcatgaataaaggatctaaggcaccattcagacatggtgattctgtgacatttacctgtaaagccaacttcaccatgaaaggaagcaaaactgtctggtgccaggcaaatgaaatgtggggaccaacagctctgccagtctgtgagagtgatttccctctggagtgcccatcacttccaacgattcataatggacaccacacaggacagcatgttgaccagtttgttgcggggttgtctgtgacatacagttgtgaacctggctatttgctcactggaaaaaagacaattaagtgcttatcttcaggagactgggatggtgtcatcccgacatgcaaagaggcccagtgtgaacatccaggaaagtttcccaatgggcaggtaaaggaacctctgagccttcaggttggcacaactgtgtacttctcctgtaatgaagggtaccaattacaaggacaaccctctagtcagtgtgtaattgttgaacagaaagccatctggactaagaagccagtatgtaaagaaattctcgaagattgtaaaggtcctcctccaagagaaaattcagaaattctctcaggctcgtggtcagaacaactatatccagaaggcacccaggctacctacaaatgccgccctggataccgaacacttggcactattgtaaaagtatgcaagaatggaaaatgggtggcgtctaacccatccaggatatgtcggaaaaagccttgtgggcatcccggagacacaccctttgggtcctttaggctggcagttggatctcaatttgagtttggtgcaaaggttgtttatacctgtgatgatgggtatcaactattaggtgaaattgattaccgtgaatgtggtgcagatggctggatcaatgatattccactatgtgaagttgtgaagtgtctacctgtgacagaactcgagaatggaagaattgtgagtggtgcagcagaaacagaccaggaatactattttggacaggtggtgcggtttgaatgcaattcaggcttcaagattgaaggacataaggaaattcattgctcagaaaatggcctttggagcaatgaaaagccacgatgtgtggaaattctctgcacaccaccgcgagtggaaaatggagatggtataaatgtgaaaccagtttacaaggagaatgaaagataccactataagtgtaagcatggttatgtgcccaaagaaagaggggatgccgtctgcacaggctctggatggagttctcagcctttctgtgaagaaaagagatgctcacctccttatattctaaatggtatctacacacctcacaggattatacacagaagtgatgatgaaatcagatatgaatgtaattatggcttctatcctgtaactggatcaactgtttcaaagtgtacacccactggctggatccctgttccaagatgtaccgaagattgtaaaggtcctcctccaagagaaaattcagaaattctctcaggctcgtggtcagaacaactatatccagaaggcacccaggctacctacaaatgccgccctggataccgaacacttggcactattgtaaaagtatgcaagaatggaaaatgggtggcgtctaacccatccaggatatgtcggaaaaagccttgtgggcatcccggagacacaccctttgggtcctttaggctggcagttggatctcaatttgagtttggtgcaaaggttgtttatacctgtgatgatgggtatcaactattaggtgaaattgattaccgtgaatgtggtgcagatggctggatcaatgatattccactatgtgaagttgtgaagtgtctacctgtgacagaactcgagaatggaagaattgtgagtggtgcagcagaaacagaccaggaatactattttggacaggtggtgcggtttgaatgcaattcaggcttcaagattgaaggacataaggaaattcattgctcagaaaatggcctttggagcaatgaaaagccacgatgtgtggaaattctctgcacaccaccgcgagtggaaaatggagatggtataaatgtgaaaccagtttacaaggagaatgaaagataccactataagtgtaagcatggttatgtgcccaaagaaagaggggatgccgtctgcacaggctctggatggagttctcagcctttctgtgaagaaaagagatgctcacctccttatattctaaatggtatctacacacctcacaggattatacacagaagtgatgatgaaatcagatatgaatgtaattatggcttctatcctgtaactggatcaactgtttcaaagtgtacacccactggctggatccctgttccaagatgtacctaa

SEQ ID NO: 20 - DNA, 2706 bp, Artificial Sequence
  source: /note=“Description of Artificial Sequence Synthetic polynucleotide”
gaattcgccgccaccatgcccatggggtctctgcaaccgctggccaccttgtacctgctggggatgctggtcgcttccgtgctagcgatttcttgtgaccctcctcctgaagtcaaaaatgctcggaaaccctattattctcttcccatagttcctggaactgttctgaggtacacttgttcacctagctaccgcctcattggagaaaaggctatcttttgtataagtgaaaatcaagtgcatgccacctgggataaagctcctcctatatgtgaatctgtgaataaaaccatttcttgctcagatcccatagtaccagggggattcatgaataaaggatctaaggcaccattcagacatggtgattctgtgacatttacctgtaaagccaacttcaccatgaaaggaagcaaaactgtctggtgccaggcaaatgaaatgtggggaccaacagctctgccagtctgtgagagtgatttccctctggagtgcccatcacttccaacgattcataatggacaccacacaggacagcatgttgaccagtttgttgcggggttgtctgtgacatacagttgtgaacctggctatttgctcactggaaaaaagacaattaagtgcttatcttcaggagactgggatggtgtcatcccgacatgcaaagaggcccagtgtgaacatccaggaaagtttcccaatgggcaggtaaaggaacctctgagccttcaggttggcacaactgtgtacttctcctgtaatgaagggtaccaattacaaggacaaccctctagtcagtgtgtaattgttgaacagaaagccatctggactaagaagccagtatgtaaagaaattctcggcggaggtgggtcgggtggcggcggatctgaagattgtaaaggtcctcctccaagagaaaattcagaaattctctcaggctcgtggtcagaacaactatatccagaaggcacccaggctacctacaaatgccgccctggataccgaacacttggcactattgtaaaagtatgcaagaatggaaaatgggtggcgtctaacccatccaggatatgtcggaaaaagccttgtgggcatcccggagacacaccctttgggtcctttaggctggcagttggatctcaatttgagtttggtgcaaaggttgtttatacctgtgatgatgggtatcaactattaggtgaaattgattaccgtgaatgtggtgcagatggctggatcaatgatattccactatgtgaagttgtgaagtgtctacctgtgacagaactcgagaatggaagaattgtgagtggtgcagcagaaacagaccaggaatactattttggacaggtggtgcggtttgaatgcaattcaggcttcaagattgaaggacataaggaaattcattgctcagaaaatggcctttggagcaatgaaaagccacgatgtgtggaaattctctgcacaccaccgcgagtggaaaatggagatggtataaatgtgaaaccagtttacaaggagaatgaaagataccactataagtgtaagcatggttatgtgcccaaagaaagaggggatgccgtctgcacaggctctggatggagttctcagcctttctgtgaagaaaagagatgctcacctccttatattctaaatggtatctacacacctcacaggattatacacagaagtgatgatgaaatcagatatgaatgtaattatggcttctatcctgtaactggatcaactgtttcaaagtgtacacccactggctggatccctgttccaagatgtaccgaagattgtaaaggtcctcctccaagagaaaattcagaaattctctcaggctcgtggtcagaacaactatatccagaaggcacccaggctacctacaaatgccgccctggataccgaacacttggcactattgtaaaagtatgcaagaatggaaaatgggtggcgtctaacccatccaggatatgtcggaaaaagccttgtgggcatcccggagacacaccctttgggtcctttaggctggcagttggatctcaatttgagtttggtgcaaaggttgtttatacctgtgatgatgggtatcaactattaggtgaaattgattaccgtgaatgtggtgcagatggctggatcaatgatattccactatgtgaagttgtgaagtgtctacctgtgacagaactcgagaatggaagaattgtgagtggtgcagcagaaacagaccaggaatactattttggacaggtggtgcggtttgaatgcaattcaggcttcaagattgaaggacataaggaaattcattgctcagaaaatggcctttggagcaatgaaaagccacgatgtgtggaaattctctgcacaccaccgcgagtggaaaatggagatggtataaatgtgaaaccagtttacaaggagaatgaaagataccactataagtgtaagcatggttatgtgcccaaagaaagaggggatgccgtctgcacaggctctggatggagttctcagcctttctgtgaagaaaagagatgctcacctccttatattctaaatggtatctacacacctcacaggattatacacagaagtgatgatgaaatcagatatgaatgtaattatggcttctatcctgtaactggatcaactgtttcaaagtgtacacccactggctggatccctgttccaagatgtacctaa

SEQ ID NO: 21 - protein, 560 aa, Artificial Sequence
  source: /note=“Description of Artificial Sequence Synthetic polypeptide”
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEEIFEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLK

SEQ ID NO: 22 - DNA, 1755 bp, Artificial Sequence
  source: /note=“Description of Artificial Sequence Synthetic polynucleotide”
gccgccaccatgggagccgctggtctgctcggcgtgttcctcgccttggtggcacctggcgtcctgggcatcagctgcggttcccctccaccaatcctgaatggcagaatctcctattactccacaccaatcgccgtcggcactgtgatcagatacagctgttcagggacttttcggctgatcggcgagaaaagcctcctctgcattaccaaggataaggtcgatgggacatgggataaaccagctcctaagtgcgagtacttcaataagtatagttcatgtccagagcccattgttcctggtggctacaagattcgggggagcacaccctatcgccacggtgactcagtgacctttgcttgtaaaaccaacttctcaatgaacggtaataagtcagtgtggtgtcaggccaataatatgtggggtcctacacgactccccacctgtgtgtccgtgttccccttggaatgccccgccctgcccatgatccataatggacaccacaccagcgagaatgtcgggagtatcgcacctggattgagtgtcacctactcatgcgagtctggctacctgcttgtaggtgaaaaaattattaattgcttgtcctccggcaaatggagtgccgttcccccaacttgtgaagaggcccggtgcaaatccctcggccgcttccctaatggtaaagttaaagagcctccaatcctcagagtgggggtgaccgctaacttcttctgtgatgaaggctaccggttgcagggaccacccagtagccggtgtgtcatagctgggcagggagtggcttggacaaagatgcccgtttgtgaggaaatcttcgaagactgtaatgagctgcccccaagacggaatacagagatcctcacaggctcttggtccgatcaaacttatccagagggtacccaggcaatttacaagtgcagacctggatacaggagcctgggcaatgtgattatggtgtgccgcaagggggagtgggtggcccttaatcctctccggaagtgtcagaaaagaccatgcggacaccctggagatacacctttcggtacctttacccttaccggcggcaatgtcttcgagtatggcgtcaaggccgtgtacacttgtaacgagggataccagctgctgggggaaataaactatcgtgagtgtgacactgacgggtggactaacgacatccccatttgcgaggtggtcaagtgccttcctgtaaccgctcccgaaaatggtaagatcgtatcttccgcaatggagcctgatcgggaataccactttggacaagccgttcggttcgtatgtaattcagggtataaaattgagggcgatgaggagatgcactgcagtgatgacggcttttggtcaaaggaaaagccaaagtgcgtagagatcagttgtaagtctcctgacgttattaacgggagtcccatcagtcagaagatcatttacaaggaaaacgagaggttccagtataaatgcaatatgggatatgagtactccgaaagaggggacgccgtgtgcacagagtccggatggcgacctttgccatcttgtgaagaaaagtcttgtgacaacccctatattcctaacggagattactctcctctgcgcatcaagcaccgaactggggacgagatcacttaccaatgtcgaaacggcttctaccctgctaccagaggtaacactgccaagtgtaccagcaccggttggattcccgcccccagatgcacacttaaatgataa

SEQ ID NO: 23 - protein, 863 aa, Artificial Sequence
  source: /note=“Description of Artificial Sequence Synthetic polypeptide”
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEEIFEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLK

SEQ ID NO: 24 - DNA, 2665 bp, Artificial Sequence
  source: /note=“Description of Artificial Sequence Synthetic polynucleotide”
cgccgccaccatgggcgcagcaggcttgttgggcgtgttcctggcattggtggcacccggcgtattgggcatttcatgcggctctcctccacccattctcaatggaaggatctcctactacagcacccccatagctgtcggcaccgttatccgatacagttgttccggtactttccggcttatcggcgaaaagtctttgctgtgcattaccaaggataaagtggacgggacttgggacaaacccgcacctaagtgcgagtattttaacaaatatagcagctgccctgagcctatagtacccggggggtataaaatccggggctctactccctatcgtcatggcgattctgtgaccttcgcatgtaaaactaatttttcaatgaatggcaacaagtctgtatggtgtcaagcaaataacatgtggggacctacccgcctgccaacctgtgtgtcagtgtttcccctggaatgtccagccctccctatgatccacaacggacatcacaccagcgaaaacgttggatccatcgcaccagggctctctgtgacttactcttgcgagtccgggtacctgctcgtgggtgaaaagatcatcaactgcctcagtagtggtaaatggtccgccgtgcctcccacatgtgaagaggcccggtgcaagagcctgggccggttccccaacggaaaagtgaaggaacctcctatcttgagggttggtgtgaccgctaactttttctgcgacgaggggtacaggctccaagggcctccctctagtcggtgcgtaatcgccggtcaaggagtcgcatggactaagatgcctgtgtgtgaggagattttcgaggattgtaatgaattgccacccaggagaaatactgaaatcctgacaggctcttggtctgatcagacttatccagaaggcacccaggccatttacaagtgtcggcctggatacagatctctgggaaatgtgatcatggtatgtaggaaaggagagtgggtggctttgaaccccctccgcaagtgtcagaaaagaccatgcgggcatcctggagacaccccattcgggacatttacactgacaggcggaaacgtatttgagtacggagtcaaggccgtttatacatgtaacgaagggtatcaactgctgggagaaatcaactatagggagtgcgacactgacggatggacaaacgacattccaatctgcgaagtggtgaaatgtcttccagttacagcccctgaaaacgggaaaatcgtgtcctccgctatggagcctgaccgggaatatcatttcggccaggccgttagattcgtgtgtaatagcggctacaaaatcgagggcgacgaagaaatgcattgcagcgatgacgggttctggagcaaggagaagcctaaatgcgtcgaaatttcatgcaagagtcccgacgtcataaacggttctccaatttcccagaagatcatttataaggagaatgagcggttccagtataagtgtaatatgggctacgagtacagcgaacgcggtgacgccgtgtgtaccgaaagtggctggagaccactgcctagttgcgaggagaaatcctgcgacaacccttatattcccaacggggactactctcctctgagaatcaagcatcggactggcgacgagattacttaccaatgcaggaacggattctatccagcaactcggggcaataccgctaagtgtacctccacaggctggatacccgctcctagatgtacagaggactgcaatgaactgccacctcggcgcaatacagaaattttgactggatcatggtctgaccagacttaccccgagggcacccaggccatctacaaatgtaggcccggttatcgaagtttgggtaacgtgattatggtgtgtcgaaaaggtgaatgggtagcactcaatcccctccgtaaatgccagaagcgtccttgtgggcacccaggcgataccccttttggaactttcaccctgactggaggaaacgtctttgaatatggtgtgaaagccgtgtacacatgcaatgaagggtaccaactgctcggagagataaactatcgggagtgcgatacagatggatggaccaatgatataccaatctgcgaggtggtgaagtgtctcccagtcaccgctcctgagaacggaaagatcgtcagttctgctatggaacctgacagggaataccactttgggcaagccgtccgcttcgtgtgcaattcagggtacaagatagaaggcgacgaagagatgcactgttccgacgatggtttctggtctaaggagaagcctaaatgtgtcgagattagctgcaagtctcccgatgttattaacggctctcccatctctcaaaaaattatttataaggaaaacgaaagatttcagtacaagtgcaatatgggttatgagtacagtgaacgtggagacgccgtgtgcacagagtccgggtggcgtccactgcccagctgcgaagaaaaatcctgtgacaacccctacatccccaatggcgactattcccccctgcgcatcaaacatcgtactggcgatgaaattacttaccagtgccgcaacgggttctaccctgccacccggggtaacacagccaaatgcacctccaccggatggatccccgccccacgctgtaccttgaaatgatga

SEQ ID NO: 25 - protein, 20 aa, Artificial Sequence
  source: /note=“Description of Artificial Sequence Synthetic peptide”
MGAAGLLGVFLALVAPGVLG

SEQ ID NO: 26 - DNA, 60 bp, Artificial Sequence
  source: /note=“Description of Artificial Sequence Synthetic oligonucleotide”
atgggagccgctggtctgctcggcgtgttcctcgccttggtggcacctggcgtcctgggc

SEQ ID NO: 27 - protein, 246 aa, Artificial Sequence
  source: /note=“Description of Artificial Sequence Synthetic polypeptide”
DIQMTQSPSSLSASVGDRVTITCGASENIYGALNWYQQKPGKAPKLLIYGATNLADGVPSRFSGSGSGTDFTLTISSLQPEDFATYYCQNVLNTPLTFGQGTKVEIKRTGGGGSGGGGSGGGGSQVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSS

SEQ ID NO: 28 - DNA, 740 bp, Artificial Sequence
  source: /note=“Description of Artificial Sequence Synthetic polynucleotide”
gatatccagatgacccagtccccgtcctccctgtccgcctctgtgggcgatagggtcaccatcacctgcggcgccagcgaaaacatctatggcgcgctgaactggtatcaacagaaacccgggaaagctccgaagcttctgatttacggtgcgacgaacctggcagatggagtcccttctcgcttctctggatccggctccggaacggatttcactctgaccatcagcagtctgcagcctgaagacttcgctacgtattactgtcagaacgttttaaatactccgttgactttcggacagggtaccaaggtggaaataaaacgtactggcggtggtggttctggtggcggtggatctggtggtggcggttctcaagtccaactggtgcaatccggcgccgaggtcaagaagccaggggcctcagtcaaagtgtcctgtaaagctagcggctatattttttctaattattggattcaatgggtgcgtcaggcccccgggcagggcctggaatggatgggtgagatcttaccgggctctggtagcaccgaatataccgaaaattttaaagaccgtgttactatgacgcgtgacacttcgactagtacagtatacatggagctctccagcctgcgatcggaggacacggccgtctattattgcgcgcgttatttttttggttctagcccgaattggtattttgatgtttggggtcaaggaaccctggtcactgtctcgagctg

SEQ ID NO: 29 - protein, 247 aa, Artificial Sequence
  source: /note=“Description of Artificial Sequence Synthetic polypeptide”
ADIQMTQSPSSLSASVGDRVTITCGASENIYGALNWYQRKPGKAPKLLIYGATNLADGVPSRFSGSGSGTDFTLTISSLQPEDFATYYCQNVLNTPLTFGQGTKVEIKRTGGGGSGGGGSGGGGSQVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSS

SEQ ID NO: 30 - protein, 448 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 31 - protein, 214 aa, Artificial Sequence [sequence omitted]

SEQ ID NO: 32 - protein, 5 aa, Artificial Sequence
  source: /note=“Description of Artificial Sequence Synthetic peptide”
GGGGS

SEQ ID NO: 33 - protein, 10 aa, Artificial Sequence
  source: /note=“Description of Artificial Sequence Synthetic peptide”
GGGGSGGGGS

SEQ ID NO: 34 - protein, 15 aa, Artificial Sequence
  source: /note=“Description of Artificial Sequence Synthetic peptide”
GGGGSGGGGSGGGGS

SEQ ID NO: 35 - protein, 16 aa, Artificial Sequence
  source: /note=“Description of Artificial Sequence Synthetic peptide”
GGGSGGGSGGGSGGGS

SEQ ID NO: 36 - protein, 5 aa, Artificial Sequence
  source: /note=“Description of Artificial Sequence Synthetic peptide”
SGGGG

SEQ ID NO: 37 - protein, 10 aa, Artificial Sequence
  source: /note=“Description of Artificial Sequence Synthetic peptide”
SGGGGSGGGG

SEQ ID NO: 38 - protein, 15 aa, Artificial Sequence
  source: /note=“Description of Artificial Sequence Synthetic peptide”
SGGGGSGGGGSGGGG

SEQ ID NO: 39 - protein, 20 aa, Artificial Sequence
  source: /note=“Description of Artificial Sequence Synthetic peptide”
SGGGGSGGGGSGGGGSGGGG

SEQ ID NO: 40 - protein, 7 aa, Artificial Sequence
  source: /note=“Description of Artificial Sequence Synthetic peptide”
VSVFPLE

SEQ ID NO: 41 - protein, 4 aa, Artificial Sequence
  source: /note=“Description of Artificial Sequence Synthetic peptide”
EEIF

SEQ ID NO: 42 - protein, 4 aa, Artificial Sequence
  source: /note=“Description of Artificial Sequence Synthetic peptide”
SFTL